(19) United States
(12) Patent Application Publication
(10) Pub. No.: US 2026/0337675 A1
(43) Pub. Date: Oct. 29, 2026

(54) ISOTHERMAL AMPLIFICATION OF NUCLEIC ACIDS

(71) Applicant: ABBOTT LABORATORIES, Abbott Park, IL (US)

(72) Inventors: Andrew Morris, Richmond, IL (US); Jessica Koranda, Vernon Hills, IL (US); Yangrong Zhang, Vernon Hills, IL (US); Kari Tanaka, Chicago, IL (US); Mario Neto, Libertyville, IL (US); Jamie Abbott, Sturtevant, WI (US); Rebecca Toroney, Chicago, IL (US); Caitlin Collins, Downers Grove, IL (US); Wei Ping Teoh, Vernon Hills, IL (US); Matthew Frankel, Libertyville, IL (US); Divya Sachdev, Wheeling, IL (US)

(73) Assignee: ABBOTT LABORATORIES, Abbott Park, IL (US)

(21) Appl. No.: 19/467,487

(22) Filed: Feb. 2, 2026

Related U.S. Application Data
(63) Continuation of application No. PCT/US2024/040969, filed on Aug. 5, 2024.
(60) Provisional application No. 63/530,676, filed on Aug. 3, 2023.
(60) Provisional application No. 63/562,603, filed on Mar. 7, 2024.

(51) Int. Cl. C12Q 1/686; C12Q 1/34; C12Q 1/48; C12Q 1/6813; C12Q 1/6876; C12Q 1/70

(52) U.S. Cl. CPC C12Q 1/686; C12Q 1/34; C12Q 1/48; C12Q 1/6813; C12Q 1/6876; C12Q 1/701; C12Y 207/03002; C12Y 207/07049; C12Y 301/00; C12Q 2600/16

(57) ABSTRACT

The present disclosure relates to the amplification of nucleic acids in a sample using isothermal application process, e.g., RPA. In particular, the present disclosure provides methods for amplifying at least one target nucleic acid in a sample, where the reagents used in the method include trehalose and polyethylene glycol (PEG). The present disclosure further provides compositions, systems and kits for performing such methods.

### CROSS-REFERENCE TO RELATED APPLICATIONS

The application is a continuation of International Application No. PCT/US2024/040969, filed Aug. 5, 2024, which claims priority to U.S. Provisional Application No. 63/530,676, filed Aug. 3, 2023, and U.S. Provisional Application No. 63/562,603, filed Mar. 7, 2024, the contents of all of which are incorporated herein by reference in their entireties.

### FIELD

The subject matter disclosed herein relates to methods for the isothermal amplification of target nucleic acids and compositions, kits and systems for performing such methods.

### BACKGROUND

The ability to amplify nucleic acids lies at the heart of modern biological and medical research. In particular, amplification of nucleic acids is commonly used in nucleic acid-based diagnostic methods for the detection of an infection, e.g., viral infection or bacterial infection, a disease and/or a genetic mutation in a subject. For example, identification of bacterial or viral nucleic acid in a blood sample can be useful in determining whether the blood is safe for donating. Other examples include the identification of specific genetic mutations for early detection of cancer or for determining the best course of treatment for a cancer.

Among the various nucleic acid amplification techniques, polymerase chain reaction (PCR) is the most common because of its sensitivity and efficiency at amplifying short nucleic acid sequences. While PCR is of great utility, it also has limitations. In particular, PCR relies on multiple cycles of thermal melting (denaturing) at high temperatures followed by hybridization and elongation at a reduced temperature (“thermal cycling”), which is a time-consuming process and requires specialized equipment. In view of the technical disadvantages of PCR and other amplification techniques that require thermal cycling, methods have been developed which enable isothermal amplification of nucleic acids.

### SUMMARY

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

To achieve these and other advantages and in accordance with the purpose of the disclosed subject matter, as embodied and broadly described, the disclosed subject matter includes methods for amplifying a target nucleic acid in a sample, e.g., isothermal amplification methods for amplifying a target nucleic acid in a sample. In certain embodiments, an isothermal amplification process of the present disclosure includes the use of a composition comprising trehalose and PEG at a trehalose to PEG weight percent (wt %) ratio of about 0.79 or less. In certain embodiments, an isothermal amplification process of the present disclosure includes the use of a composition comprising trehalose and PEG at a trehalose to PEG weight percent (wt %) ratio of about 0.75 or less. In certain embodiments, an isothermal amplification process of the present disclosure includes the use of a composition comprising trehalose and PEG at a trehalose to PEG weight percent (wt %) ratio of about 0.70 or less. In certain embodiments, an isothermal amplification process of the present disclosure includes the use of a composition comprising trehalose and PEG at a trehalose to PEG weight percent (wt %) ratio of about 0.65 or less. In certain embodiments, an isothermal amplification process of the present disclosure includes the use of a composition comprising trehalose and PEG at a trehalose to PEG weight percent (wt %) ratio of about 0.60 or less. In certain embodiments, an isothermal amplification process of the present disclosure includes the use of a composition comprising trehalose and PEG at a trehalose to PEG weight percent (wt %) ratio of about 0.55 or less.

The present disclosure provides an isothermal amplification process for amplifying a target nucleic acid that includes (i) preparing a reagent composition using a composition comprising trehalose and a polyethylene glycol (PEG), where the trehalose and PEG are present in the composition at a trehalose to PEG weight percent (wt %) ratio of about 0.79 or less and (ii) contacting the target nucleic acid with the reagent composition to amplify the target nucleic acid. In certain embodiments, the method can include (i) providing a reagent composition, where the reagent composition is prepared using a composition comprising trehalose and a polyethylene glycol (PEG), and where the trehalose and PEG are present in the composition at a trehalose to PEG weight percent (wt %) ratio of about 0.79 or less and (ii) contacting the target nucleic acid with the reagent composition to amplify the target nucleic acid.

In certain embodiments, the trehalose to PEG wt % ratio is about 0.25 to about 0.79 in the composition. In certain embodiments, the trehalose to PEG wt % ratio is about 0.25 to about 0.75 in the composition. In certain embodiments, the trehalose to PEG wt % ratio is about 0.30 to about 0.70 in the composition. In certain embodiments, the trehalose to PEG wt % ratio is about 0.30 to about 0.60 in the composition. In certain embodiments, the trehalose to PEG wt % ratio is about 0.30 to about 0.55 in the composition. In certain embodiments, the PEG is present in the composition at an amount of about 10% to about 30% weight to volume (w/v). In certain embodiments, the PEG is present in the composition at an amount of about 15% to about 20% weight to volume (w/v). In certain embodiments, the reagent composition comprises PEG at an amount from about 2% to about 8% weight to volume (w/v). In certain embodiments, the trehalose is present at the composition in an amount of about 5% to about 20% w/v. In certain embodiments, the trehalose is present in the composition at an amount of about 5% to about 15% w/v. In certain embodiments, the trehalose is present in the composition at an amount of about 5% to about 12% w/v. In certain embodiments, the reagent composition comprises trehalose at an amount of about 3% to about 12% w/v. In certain embodiments, the reagent composition comprises trehalose at an amount of about 3% to about 10% w/v.

In certain embodiments, the composition further comprises one or more of ATP, dNTPs, phosphocreatine and one or more salts. In certain embodiments, the composition further comprises ATP, dNTPs, phosphocreatine and one or more salts. In certain embodiments, the reagent composition further comprises one or more of a DNA polymerase, a recombinase loading protein, a single stranded binding protein, ATP, dNTPs or a mixture of dNTPs and ddNTPs, a reducing agent, creatine kinase, a nuclease (e.g., an exonuclease), one or more primers, a nucleic acid probe and a reverse transcriptase.

In certain embodiments, an isothermal amplification process of the present disclosure includes an isothermal amplification process that is performed in the presence of a trehalose to PEG weight percent (wt %) ratio of about 0.79 or less. In certain embodiments, the method includes an isothermal amplification process that is performed in the presence of a trehalose to PEG weight percent (wt %) ratio of about 0.75 or less.

The present disclosure further provides an isothermal amplification process for amplifying a target nucleic acid that includes contacting the target nucleic acid (or a sample comprising the target nucleic acid) with a reagent composition comprising trehalose and a polyethylene glycol (PEG) to amplify the target nucleic acid, wherein the trehalose and PEG are at a weight percent (wt %) ratio of about 0.79 or less. In certain embodiments, the trehalose to PEG wt % ratio is about 0.75 or less. In certain embodiments, the trehalose to PEG wt % ratio is about 0.70 or less. In certain embodiments, the trehalose to PEG wt % ratio is about 0.30 to about 0.79. In certain embodiments, the trehalose to PEG wt % ratio is about 0.40 to about 0.79. In certain embodiments, the trehalose to PEG wt % ratio is about 0.45 to about 0.75. In certain embodiments, the trehalose to PEG wt % ratio is about 0.50 to about 0.70. In certain embodiments, the trehalose to PEG wt % ratio is about 0.50 to about 0.60. In certain embodiments, the trehalose to PEG wt % ratio is about 0.30 to about 0.50. In certain embodiments, the trehalose to PEG wt % ratio is about 0.30 to about 0.45.

In certain embodiments, the PEG is present in an isothermal amplification process and/or reagent composition at an amount of about 2% to about 9% weight to volume (w/v), e g., about 2% to about 6% w/v, about 2% to about 6% w/v or about 6% to about 8% w/v. In certain embodiments, the PEG is present in an isothermal amplification process and/or reagent composition at an amount of about 2% to about 8% weight to volume (w/v). In certain embodiments, the PEG has a molecular weight between about 20,000 Daltons to about 50,000 daltons, e.g., the PEG has a molecular weight of about 35,000 daltons.

In certain embodiments, the trehalose is present in an isothermal amplification process and/or reagent composition at an amount of about 1% to about 12% w/v. In certain embodiments, the trehalose is present in the isothermal amplification process and/or reagent composition at an amount of about 2% to about 12% w/v. In certain embodiments, the trehalose is present in the isothermal amplification process and/or reagent composition at an amount of about 3% to about 12% w/v. In certain embodiments, the trehalose is present in the isothermal amplification process and/or reagent composition at an amount of about 5% to about 9% w/v. In certain embodiments, the trehalose is present in the isothermal amplification process and/or reagent composition at an amount of about 3% to about 5% w/v. In certain embodiments, the trehalose is present in the isothermal amplification process and/or reagent composition at an amount of about 2% w/v. In certain embodiments, the trehalose is present in the isothermal amplification process and/or reagent composition at an amount of about 3% w/v. In certain embodiments, the trehalose is present in the isothermal amplification process and/or reagent composition at an amount of about 4% w/v. In certain embodiments, the trehalose is present in the isothermal amplification process and/or reagent composition at an amount of about 6% w/v.

In certain embodiments, the isothermal amplification process further includes contacting the target nucleic acid (or the sample comprising the target nucleic acid) with one or more of a DNA polymerase, a recombinase, a recombinase loading protein, a single stranded binding protein, ATP, dNTPs or a mixture of dNTPs and ddNTPs, a reducing agent, creatine kinase, a nuclease (e.g., an exonuclease), one or more primers, a nucleic acid probe, a reverse transcriptase and an activator.

In certain embodiments, the reagent composition further includes one or more of a DNA polymerase, a recombinase, a recombinase loading protein, a single stranded binding protein, ATP, dNTPs or a mixture of dNTPs and ddNTPs, a reducing agent, creatine kinase, a nuclease (e.g., an exonuclease), one or more primers, a nucleic acid probe and a reverse transcriptase.

In certain embodiments, the isothermal amplification process is selected from the group consisting of rolling circle amplification (RCA), nucleic acid sequence-based amplification (NASBA), strand displacement amplification (SDA), Transcription-Mediated Amplification (TMA), Single Primer Isothermal Amplification (SPIA), Helicase-dependent amplification (HDA), Loop mediated amplification (LAMP), Recombinase-Polymerase Amplification (RPA) and Nicking Enzyme Amplification Reaction (NEAR). In certain embodiments, the isothermal amplification process is RPA. In certain embodiments, the isothermal amplification process is NEAR.

In certain embodiments, the target nucleic acid is a bacterial, eukaryotic or viral nucleic acid. In certain embodiments, the target nucleic acid is derived from SARS-COV-2 (COVID-19), HIV-1, HIV-2, HBV, HCV, CMV, Parvovirus B19, HAV, Chlamydia, Gonorrhea, WNV, Zika Virus, Dengue Virus, Chikungunya Virus, Influenza, Babesia, Malaria, Usutu Virus or HEV.

In certain embodiments, the isothermal amplification process amplifies at least two target nucleic acids or at least three target nucleic acids, e.g., in a multiplex reaction.

In certain embodiments, the target nucleic acid is present in a sample and/or obtained from a sample. In certain embodiments, the sample is a tissue sample. In certain embodiments, the target nucleic acid is isolated from the tissue sample prior to amplification.

In certain embodiments, the sample is a biological fluid. In certain embodiments, the biological fluid is blood, e.g., whole blood, lysed whole blood, serum or plasma. In certain embodiments, the target nucleic acid is isolated from the biological fluid prior to amplification.

The present disclosure further provides a composition for performing an isothermal amplification process. In certain embodiments, the composition includes trehalose and PEG at a weight percent (wt %) ratio of about 0.79 or less. In certain embodiments, the composition includes trehalose and PEG at a wt % ratio of about 0.75 or less. In certain embodiments, the composition includes trehalose and PEG at a wt % ratio of about 0.70 or less. In certain embodiments, the composition includes trehalose and PEG at a wt % ratio of about 0.3 to about 0.79. In certain embodiments, the composition includes trehalose and PEG at a wt % ratio of about 0.4 to about 0.79. In certain embodiments, the composition includes trehalose and PEG at a wt % ratio of about 0.45 to about 0.75. In certain embodiments, the composition includes trehalose and PEG at a wt % ratio of about 0.50 to about 0.70. In certain embodiments, the composition includes trehalose and PEG at a wt % ratio of about 0.50 to about 0.60. In certain embodiments, the composition includes trehalose and PEG at a wt % ratio of about 0.30 to about 0.50. In certain embodiments, the composition includes trehalose and PEG at a wt % ratio of about 0.30 to about 0.45.

In certain embodiments, a composition of the present disclosure includes PEG in an amount of about 2% to about 9% weight to volume (w/v), e g., about 2% to about 6% w/v, about 2% to about 6% w/v or about 6% to about 8% w/v. In certain embodiments, the PEG has a molecular weight between about 20,000 Daltons to about 50,000 daltons, e.g., the PEG has a molecular weight of about 35,000 daltons.

In certain embodiments, a composition of the present disclosure includes trehalose in an amount of about 1% to about 12% w/v. In certain embodiments, the composition includes trehalose in an amount of about 2% to about 12% w/v. In certain embodiments, the composition includes trehalose in an amount of about 4% to about 9% w/v. In certain embodiments, the composition includes trehalose in an amount of about 3% to about 5% w/v. In certain embodiments, the trehalose is present in the isothermal amplification process and/or reagent composition at an amount of about 2% w/v. In certain embodiments, the trehalose is present in the isothermal amplification process and/or reagent composition at an amount of about 3% w/v. In certain embodiments, the composition includes trehalose in an amount of about 4% w/v. In certain embodiments, the trehalose is present in the isothermal amplification process and/or reagent composition at an amount of about 6% w/v.

In certain embodiments, the composition further includes one or more of ATP, dNTPs or a mixture of dNTPs and ddNTPs. In certain embodiments, the composition further includes one or more of ATP, dNTPs or a mixture of dNTPs and ddNTPs and a reducing agent. In certain embodiments, the composition further includes one or more salts and/or phosphocreatine. In certain embodiments, the composition further includes one or more of a DNA polymerase, a recombinase, a recombinase loading protein, a single stranded binding protein, ATP, dNTPs or a mixture of dNTPs and ddNTPs, a reducing agent, creatine kinase, a nuclease, one or more primers, a nucleic acid probe and a reverse transcriptase.

The present disclosure provides a composition for use in an isothermal amplification process that comprises or consists essentially of trehalose, PEG, ATP, dNTPs, phosphocreatine and one or more salts, wherein trehalose and PEG are present in the composition at a weight percent (wt %) ratio of about 0.79 or less. In certain embodiments, a composition for use in an isothermal amplification process comprises or consists essentially of trehalose, PEG, ATP, dNTPs, phosphocreatine and one or more salts, wherein trehalose and PEG are present in the composition at a wt % ratio of about 0.75 or less. In certain embodiments, a composition for use in an isothermal amplification process comprises or consists essentially of trehalose, PEG, ATP, dNTPs, phosphocreatine and one or more salts, where trehalose and PEG are present in the composition at a wt % ratio of about 0.70 or less. In certain embodiments, a composition for use in an isothermal amplification process comprises or consists essentially of trehalose, PEG, ATP, dNTPs, phosphocreatine and one or more salts, where trehalose and PEG are present in the composition at a weight percent (wt %) ratio of from about 0.25 to about 0.79. In certain embodiments, a composition for use in an isothermal amplification process comprises or consists essentially of trehalose, PEG, ATP, dNTPs, phosphocreatine and one or more salts, where trehalose and PEG are present in the composition at a wt % ratio of about 0.30 to about 0.79. In certain embodiments, a composition for use in an isothermal amplification process comprises or consists essentially of trehalose, PEG, ATP, dNTPs, phosphocreatine and one or more salts, where trehalose and PEG are present in the composition at a wt % ratio of about 0.40 to about 0.79. In certain embodiments, a composition for use in an isothermal amplification process comprises or consists essentially of trehalose, PEG, ATP, dNTPs, phosphocreatine and one or more salts, where trehalose and PEG are present in the composition at a wt % ratio of about 0.45 to about 0.75. In certain embodiments, a composition for use in an isothermal amplification process comprises or consists essentially of trehalose, PEG, ATP, dNTPs, phosphocreatine and one or more salts, where trehalose and PEG are present in the composition at a wt % ratio of about 0.50 to about 0.70. In certain embodiments, a composition for use in an isothermal amplification process comprises or consists essentially of trehalose, PEG, ATP, dNTPs, phosphocreatine and one or more salts, where trehalose and PEG are present in the composition at a wt % ratio of about 0.50 to about 0.60. In certain embodiments, a composition for use in an isothermal amplification process comprises or consists essentially of trehalose, PEG, ATP, dNTPs, phosphocreatine and one or more salts, where trehalose and PEG are present in the composition at a wt % ratio of about 0.30 to about 0.50. In certain embodiments, a composition for use in an isothermal amplification process comprises or consists essentially of trehalose, PEG, ATP, dNTPs, phosphocreatine and one or more salts, where trehalose and PEG are present in the composition at a wt % ratio of about 0.30 to about 0.45.

In certain embodiments, the composition is for use in an isothermal amplification process selected from the group consisting of rolling circle amplification (RCA), nucleic acid sequence-based amplification (NASBA), strand displacement amplification (SDA), Transcription-Mediated Amplification (TMA), Single Primer Isothermal Amplification (SPIA), Helicase-dependent amplification (HDA), Loop mediated amplification (LAMP), Recombinase-Polymerase Amplification (RPA) and Nicking Enzyme Amplification Reaction (NEAR). In certain embodiments, the isothermal amplification process is RPA. In certain embodiments, the isothermal amplification process is NEAR.

In another aspect, the present disclosure provides a system for performing the methods described herein. In certain embodiments, the system is an automated system. In certain embodiments, the system includes a container or reservoir comprising at least one composition that includes trehalose and PEG at a weight percent (wt %) ratio of about 0.79 or less. In certain embodiments, the system includes a container or reservoir comprising at least one composition that includes trehalose and PEG at a wt % ratio of about 0.78 or less. In certain embodiments, the system includes a container or reservoir comprising at least one composition that includes trehalose and PEG at a wt % ratio of about 0.75 or less. In certain embodiments, the system includes a container or reservoir comprising at least one composition that includes trehalose and PEG at a wt % ratio of about 0.70 or less. In certain embodiments, the system includes a container or reservoir comprising at least one composition that includes trehalose and PEG at a wt % ratio of about 0.30 to about 0.79. In certain embodiments, the system includes a container or reservoir comprising at least one composition that includes trehalose and PEG at a wt % ratio of about 0.40 to about 0.79. In certain embodiments, the system includes a container or reservoir comprising at least one composition that includes trehalose and PEG at a wt % ratio of about 0.45 to about 0.75. In certain embodiments, the system includes a container or reservoir comprising at least one composition that includes trehalose and PEG at a wt % ratio of about 0.50 to about 0.70. In certain embodiments, the system includes a container or reservoir comprising at least one composition that includes trehalose and PEG at a wt % ratio of about 0.50 to about 0.60. In certain embodiments, the system includes a container or reservoir comprising at least one composition that includes trehalose and PEG at a wt % ratio of about 0.30 to about 0.50. In certain embodiments, the system includes a container or reservoir comprising at least one composition that includes trehalose and PEG at a wt % ratio of about 0.30 to about 0.45.

The present disclosure further provides kits for performing the methods described herein.

The present disclosure provides kits including the compositions described herein. In certain embodiments, the kit includes a composition comprising trehalose and PEG at a weight percent (wt %) ratio of about 0.79 or less. For example, but not by way of limitation, the kit includes a composition comprising trehalose and PEG at a weight percent (wt %) ratio of about 0.75 or less. In certain embodiments, the kit includes a composition comprising trehalose and PEG at a weight percent (wt %) ratio from about 0.3 to about 0.79. In certain embodiments, the kit includes a composition comprising trehalose and PEG at a weight percent (wt %) ratio from about 0.40 to about 0.79. In certain embodiments, the kit includes a composition comprising trehalose and PEG at a weight percent (wt %) ratio from about 0.45 to about 0.75. In certain embodiments, the kit includes a composition comprising trehalose and PEG at a weight percent (wt %) ratio from about 0.50 to about 0.70. In certain embodiments, the kit includes a composition comprising trehalose and PEG at a weight percent (wt %) ratio from about 0.50 to about 0.60. In certain embodiments, the kit includes a composition comprising trehalose and PEG at a weight percent (wt %) ratio from about 0.30 to about 0.50. In certain embodiments, the kit includes a composition comprising trehalose and PEG at a weight percent (wt %) ratio from about 0.30 to about 0.45.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations and equivalents in form and function, without departing from the scope of this disclosure.

FIG. 1 provides exemplary results associated with RPA amplification of HCV and HIV-2 target nucleic acids with the addition of 4% trehalose in combination with polyethylene glycol (PEG).

FIG. 2 provides exemplary results associated with RPA amplification of HCV and internal control (IC) target nucleic acids at near Limit of Detection (LOD) levels with the addition of 4% trehalose in combination with PEG.

FIGS. 3A and 3B provide exemplary results of RPA amplification of Chikungunya (CHIKV) and Dengue (DENV) with the addition of trehalose and PEG.

FIG. 4 provides exemplary results associated with RPA amplification of HCV target nucleic acids with increasing trehalose concentration.

FIGS. 5A-5C provide exemplary results associated with RPA amplification of HBV (FIG. 5A), HCV (FIG. 5B) and an internal control (IC; FIG. 5C) targets with increasing PEG concentration.

FIGS. 6A and 6B provide exemplary results associated with RPA amplification of DENV (FIG. 6A) and an elution buffer negative control (“EB only”) (FIG. 6B) with increasing PEG concentration.

### DETAILED DESCRIPTION

The present disclosure provides improved methods of amplifying and detecting nucleic acids in a sample using isothermal amplification processes, e.g., Recombinase Polymerase Amplification (RPA). The present disclosure further provides compositions, systems and kits for performing isothermal amplification reactions.

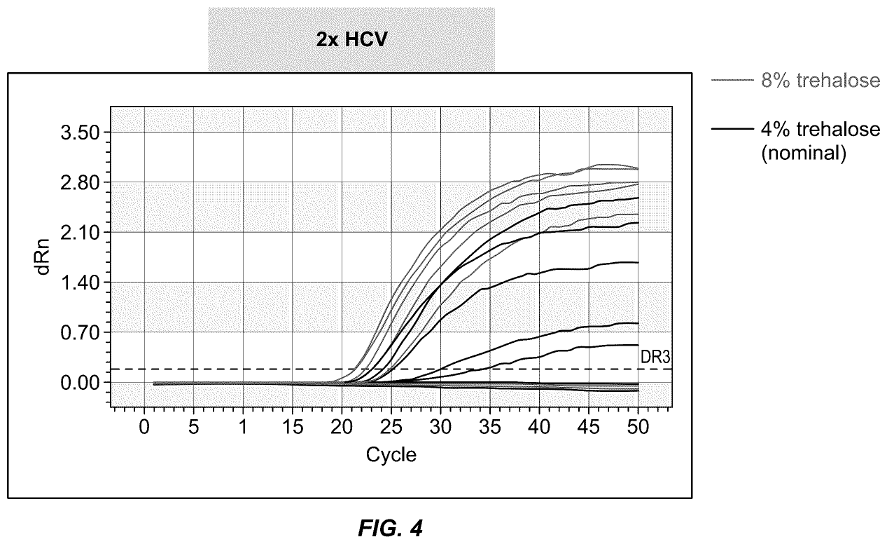

The present disclosure is based, in part, on the observation that the presence of trehalose and polyethylene glycol (PEG) at specific weight percent (wt %) ratios and specific amounts in a composition for use in an RPA reaction can significantly improve the amplification of target nucleic acids. As shown in Example 1, the use of a reagent composition (e.g., a reagent composition that includes non-protein components) that includes trehalose and PEG at wt % ratios from about 0.3 to about 0.79, e.g., about 0.5 to about 0.75, resulted in a significant increase in the amplification of target nucleic acids, including target nucleic acids of HBV, HCV, HIV, HEV, Chikungunya and Dengue, and multiplex amplification of target nucleic acids. In particular, as shown in FIGS. 4-6A, amplification of target nucleic acid was significantly improved in the RPA reactions with different trehalose and PEG ratios.

For clarity, but not by way of limitation, the detailed description of the presently disclosed subject matter is divided into the following subsections:
I. Definitions;
II. Isothermal Amplification Processes;
III. Compositions;
IV. Methods of Use;
V. Kits;
VI. Systems; and
VII. Exemplary Embodiments.

### I. DEFINITIONS

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

As used herein, the use of the word “a” or “an” when used in conjunction with the term “comprising” in the claims and/or the specification can mean “one,” but it is also consistent with the meaning of “one or more,” “at least one,” and “one or more than one.” For example, but not by way of limitation, reference to “an” or “the” “target nucleic acid” encompasses a single target nucleic acid, as well as a combination and/or mixture of two or more different target nucleic acids.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value.

As used herein, the term “amplified” refers to the process of making multiple copies of the nucleic acid from a single or lower copy number of nucleic acid molecules. The amplified nucleic acid can be referred to as an amplicon.

The term “amplification process” refers generally to any process where a portion of a nucleic acid is copied or replicated into at least one additional nucleic acid molecule. An amplification process produces amplified nucleic acids.

The term “biological fluid,” as used herein, refers to any bodily fluid or bodily fluid derivative in which the analyte can be measured. Non-limiting examples of a biological fluid include dermal fluid, interstitial fluid, plasma, blood, lymph, synovial fluid, cerebrospinal fluid, saliva, bronchoalveolar lavage, amniotic fluid, sweat, tears, or the like. In certain embodiments, the biological fluid is blood.

The terms “comprise(s),” “include(s),” “having,” “has,” “can,” “contain(s),” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

The term “coupled” can refer to the connecting or uniting of two or more components by an interaction, bond, link, force or tie in order to keep two or more components together. In certain embodiments, the term “coupled” encompasses either direct or indirect binding where, for example, a first component is directly bound to a second component, or one or more intermediate molecules are disposed between the first component and the second component. Exemplary bonds comprise covalent bonds, ionic bonds, van der Waals interactions and other bonds identifiable by a skilled person.

The terms “detect” or “detection,” as used herein, indicates the determination of the existence and/or presence of a target nucleic acid in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of the presence or absence of a target or signal, without dependence on the quantity or amount of the target or signal beyond its presence or absence.

The terms “expression” or “expresses,” as used herein, refer to transcription and translation occurring within a cell. The level of expression of a gene and/or nucleic acid in a cell can be determined on the basis of either the amount of corresponding mRNA that is present in the cell or the amount of the protein encoded by the gene and/or nucleic acid that is produced by the cell. For example, mRNA transcribed from a gene and/or nucleic acid is desirably quantitated by northern hybridization. Sambrook et al., Molecular Cloning: A Laboratory Manual, pp. 7.3-7.57 (Cold Spring Harbor Laboratory Press, 1989). Protein encoded by a gene and/or nucleic acid can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

As used herein, the term “hybridization,” refers to the process in which two single-stranded polynucleotides bind non-covalently to form a stable double-stranded polynucleotide.

As used herein, a “label” refers to an agent that allows for direct or indirect detection. Labels include, but are not limited to, fluorescent labels, chromogenic labels, electron dense labels, chemiluminescent labels and radioactive labels. Non-limiting examples of labels include green fluorescent protein (“GFP”), mCherry, dtTomato, or other fluorescent proteins known in the art (e.g., Shaner et al., A Guide to Choosing Fluorescent Proteins, Nature Methods 2(12):905-909 (2005) incorporated by reference herein, ³²P, ¹⁴C, ¹²⁵I, ³H and ¹³¹I, fluorogens (such as Rare Earth Chelate or lucifer yellow and its derivatives), Rhodamine (rhodamine) and its derivatives, dansyl, umbelliferone, luciferase (such as firefly luciferase and bacterial fluorescence plain enzyme) (U.S. Pat. No. 4,737,456), fluorescein, 2,3-dihydros phthalazine diketone, as well as enzymes producing detectable signals, e.g., horseradish peroxidase (HRP), alkaline phosphorus sour enzyme, beta galactosidase, glucoamylase, lysozyme, carbohydrate oxidase (such as glucose oxidase, galactose oxidase and glucose-6-phosphate dehydrogenase (G6PD)) and heterocyclic oxidases (such as uricase and xanthine oxidase).

The term “nucleic acid,” “nucleic acid molecule” or “polynucleotide” as used herein refers to any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby the bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. In certain embodiments, the nucleic acid is isolated. In certain embodiments, the term “isolated nucleic acid” can refer to a nucleic acid removed from a subject or a sample, e.g., removed from its original environment (e.g., the natural environment or a host cell if recombinantly expressed).

The term “oligonucleotide,” as used herein, refers to a short nucleic acid sequence comprising from about 2 to about 100 nucleotides (e.g., about 5, 10, 15, 20, 25, 30, 35, 40, 45, 50, 55, 60, 65, 70, 75, 80, 85, 90, 95, 99 or 100 nucleotides, or a range defined by any of the foregoing values). The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, for example, methylated and/or capped polynucleotides. Nucleic acids are typically linked via phosphate bonds to form nucleic acid sequences or polynucleotides, though many other linkages are known in the art (e.g., phosphorothioates, boranophosphates, and the like).

Oligonucleotides can be single-stranded or double-stranded or can contain portions of both double-stranded and single-stranded sequences. The oligonucleotide can be DNA, both genomic and complimentary DNA (cDNA), RNA, or a hybrid, where the nucleic acid can contain combinations of deoxyribo- and ribonucleotides, and combinations of bases including uracil, adenine, thymine, cytosine, guanine, inosine, xanthine hypoxanthine, isocytosine and isoguanine. Oligonucleotides can be obtained by chemical synthesis methods or by recombinant methods.

Any of the oligonucleotides described herein can be modified in any suitable manner so as to stabilize or enhance the binding affinity of the oligonucleotide for its target. For example, an oligonucleotide sequence as described herein can comprise one or more modified oligonucleotide bases.

Any of the oligonucleotide sequences described herein can comprise, consist essentially of, or consist of a complement of any of the sequences disclosed herein. The terms “complement” or “complementary sequence,” as used herein, refer to a nucleic acid sequence that forms a stable duplex with an oligonucleotide described herein via Watson-Crick base pairing rules, and typically shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% greater identity with the disclosed oligonucleotide.

The oligonucleotides described herein can be prepared using any suitable method, a variety of which are known in the art (see, for example, Sambrook et al., Molecular Cloning. A Laboratory Manual, 1989, 2. Supp. Ed., Cold Spring Harbour Laboratory Press: New York, N.Y.; M. A. Innis (Ed.), PCR Protocols. A Guide to Methods and Applications, Academic Press: New York, N.Y. (1990); P. Tijssen, Hybridization with Nucleic Acid Probes—Laboratory Techniques in Biochemistry and Molecular Biology (Parts I and II), Elsevier Science (1993); M. A. Innis (Ed.), PCR Strategies, Academic Press: New York, N.Y. (1995); and F. M. Ausubel (Ed.), Short Protocols in Molecular Biology, John Wiley & Sons: Secaucus, N. J. (2002); Narang et al., Meth. Enzymol., 68:90-98 (1979); Brown et al., Meth. Enzymol., 68:109-151 (1979); and Belousov et al., Nucleic Acids Res., 25:3440-3444 (1997), each of which is incorporated herein by reference in its entirety). Oligonucleotide pairs also can be designed using a variety of tools, such as the Primer-BLAST tool provided by the National Center of Biotechnology Information (NCBI). Oligonucleotide synthesis can be performed on oligo synthesizers such as those commercially available from Perkin Elmer/Applied Biosystems, Inc. (Foster City, CA), DuPont (Wilmington, DE), or Milligen (Bedford, MA). Alternatively, oligonucleotides can be custom made and obtained from a variety of commercial sources well-known in the art, including, for example, the Midland Certified Reagent Company (Midland, TX), Eurofins Scientific (Louisville, KY), BioSearch Technologies, Inc. (Novato, CA), and the like. Oligonucleotides can be purified using any suitable method known in the art, such as, for example, native acrylamide gel electrophoresis, anion-exchange HPLC (see, e.g., Pearson et al., J. Chrom., 255:137-149 (1983), incorporated herein by reference), and reverse phase HPLC (see, e.g., McFarland et al., Nucleic Acids Res., 7:1067-1080 (1979), incorporated herein by reference).

The sequence of the oligonucleotides can be verified using any suitable sequencing method known in the art, including, but not limited to, chemical degradation (see, e.g., Maxam et al., Methods of Enzymology, 65:499-560 (1980), incorporated herein by reference), matrix-assisted laser desorption ionization time-of-flight (MALDI-TOF) mass spectrometry (see, e.g., Pieles et al., Nucleic Acids Res., 21:3191-3196 (1993), incorporated herein by reference), mass spectrometry following a combination of alkaline phosphatase and exonuclease digestions (Wu et al., Anal. Biochem., 290:347-352 (2001), incorporated herein by reference), and the like.

The term “plurality” refers to a number larger than one. In certain embodiments, the term “plurality of nucleic acids” refers to a number of nucleic acids larger than one. For example, but not by way of limitation, a plurality of target nucleic acids includes at least two target nucleic acids.

The terms “primer,” “primer sequence,” “primer oligonucleotide,” and “amplification oligonucleotide” as used herein, refer to an oligonucleotide which is capable of acting as a point of initiation of synthesis of an extension product that is a complementary strand of nucleic acid (all types of DNA or RNA) when placed under suitable amplification conditions (e.g., buffer, salt, temperature and pH) in the presence of nucleotides and an agent for nucleic acid polymerization (e.g., a DNA-dependent or RNA-dependent polymerase). The amplification oligonucleotides of the present disclosure can be of any suitable size, and desirably comprise, consist essentially of, or consist of about 15 to 50 nucleotides, preferably about 20 to 40 nucleotides. The oligonucleotides of the present disclosure can contain additional nucleotides in addition to those described herein.

The terms “probe,” “probe sequence,” and “probe oligonucleotide,” refer to an oligonucleotide that can selectively hybridize to at least a portion of a target sequence (e.g., a portion of a target sequence that has been amplified) under appropriate hybridization conditions. In general, a probe sequence is identified as being either “complementary” (i.e., complementary to the coding or sense strand (+)), or “reverse complementary” (i.e., complementary to the anti-sense strand (−)). The probes of the present disclosure can be of any suitable size, and desirably comprise, consist essentially of, or consist of about 10-50 nucleotides, preferably about 12-35 nucleotides.

As used herein, the terms “set,” “primer set,” “probe set,” and “primer and probe set,” refer to two or more oligonucleotides which together are capable of priming the amplification of a target sequence or target nucleic acid of interest (e.g., a target sequence within an infectious agent) and/or at least one probe which can detect the target sequence or target nucleic acid. In certain embodiments, the term “set” refers to a pair of oligonucleotides including a first oligonucleotide, referred herein as a “forward primer” that hybridizes with the 5′-end of the target sequence or target nucleic acid to be amplified and a second oligonucleotide, referred herein as a “reverse primer” that hybridizes with the complement of the target sequence or target nucleic acid to be amplified.

As used herein, “reference sequence” is a defined sequence used as a basis for sequence comparison. A reference sequence can be a subset or the entirety of a specified sequence; for example, as a segment of a full-length protein or protein fragment. A reference sequence can be, for example, a sequence identifiable in a database such as GenBank and UniProt and others identifiable to those skilled in the art.

As used herein, “sequence identity” or “identity” in the context of two polynucleotide or polypeptide sequences makes reference to the nucleotide bases or amino acid residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

As used herein, “percentage of sequence identity” or “percentage of identity” means the value determined by comparing two optimally aligned sequences over a comparison window, wherein the portion of the polynucleotide sequence in the comparison window can include additions or deletions (gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As understood by those skilled in the art, determination of percent identity between any two sequences can be accomplished using certain well-known mathematical algorithms. Non-limiting examples of such mathematical algorithms are the algorithm of Myers and Miller, the local homology algorithm of Smith et al.; the homology alignment algorithm of Needleman and Wunsch; the search-for-similarity-method of Pearson and Lipman; the algorithm of Karlin and Altschul, modified as in Karlin and Altschul. Computer implementations of suitable mathematical algorithms can be utilized for comparison of sequences to determine sequence identity. Such implementations include, but are not limited to: CLUSTAL, ALIGN, GAP, BESTFIT, BLAST, FASTA, among others identifiable by skilled persons. Sequence alignment algorithms also are disclosed in, for example, Altschul et al., J. Molecular Biol., 215 (3): 403-410 (1990); Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009); Soding, Bioinformatics, 21(7): 951-960 (2005); Altschul et al., Nucleic Acids Res., 25(17): 3389-3402 (1997); and Gusfield, Algorithms on Strings, Trees and Sequences, Cambridge University Press, Cambridge UK (1997), each of which is incorporated herein by reference in its entirety).

As used herein, the term “subject” or “individual” refers to a vertebrate or an invertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, guinea pigs, rabbits, dogs, cats, sheep, pigs, goats, cattle, horses, apes and monkeys. In certain embodiments, the individual or subject is a human.

The terms “target nucleic acid”, “target sequence”, or “target nucleic acid sequence,” as used herein, refers to a nucleic acid sequence of interest to be amplified using the methods of the present disclosure.

### II. Isothermal Amplification Processes

The presently disclosed subject matter provides methods of amplifying target nucleic acids in a sample. In certain embodiments, the amplification process is an isothermal amplification process. Isothermal amplification processes include amplification processes that do not require temperature cycling or rapid heating and cooling for amplification of a target nucleic acid to occur.

Non-limiting examples of isothermal amplification processes include rolling circle amplification (RCA), nucleic acid sequence-based amplification (NASBA), strand displacement amplification (SDA), Transcription-Mediated Amplification (TMA), Single Primer Isothermal Amplification (SPIA), Helicase-dependent amplification (HDA), Loop mediated amplification (LAMP), Recombinase-Polymerase Amplification (RPA) and Nicking Enzyme Amplification Reaction (NEAR). Additional non-limiting disclosure regarding isothermal amplification methods is provided in Oliveira et al., Frontiers in Sensors 2:752600 (2021), the contents of which is incorporated herein by reference in its entirety.

In certain embodiments, the isothermal amplification process is RPA.

In certain embodiments, the isothermal amplification process is NEAR.

The present disclosure provides isothermal amplification processes that are performed in the presence of certain weight percent (wt %) ratios of trehalose to PEG and/or uses compositions that have certain weight percent (wt %) ratios of trehalose to PEG. As shown in Example 1, wt % ratios of trehalose to PEG of about 0.31, about 0.52, about 0.62, about 0.72 and about 0.73, e.g., in the non-protein component (NPC) composition, resulted in significant improvement of the RPA process compared to other ratios analyzed. In certain embodiments, the addition of trehalose to the NPC composition that includes PEG allows for an increase in the concentration of crowding agents without affecting the viscosity of the NPC composition. In certain embodiments, increasing the viscosity of the NPC can make the composition unworkable or challenging to use.

In certain embodiments, the isothermal amplification process is performed in the presence of a trehalose to PEG wt % ratio and/or uses a composition that includes trehalose and PEG at a wt % ratio of about 0.25 to about 0.79 or about 0.3 to about 0.79, e.g., about 0.4 to about 0.75. In certain embodiments, the isothermal amplification process is performed in the presence of a trehalose to PEG wt % ratio and/or uses a composition that includes trehalose and PEG at a wt % ratio of about 0.3 to about 0.7, about 0.3 to about 0.65, about 0.3 to about 0.6, about 0.3 to about 0.55, about 0.3 to about 0.45, about 0.3 to about 0.4, about 0.45 to about 0.75, about 0.5 to about 0.75, about 0.55 to about 0.75, about 0.6 to about 0.75, about 0.65 to about 0.75, about 0.7 to about 0.75, about 0.4 to about 0.7, about 0.4 to about 0.65, about 0.4 to about 0.6, about 0.4 to about 0.55, about 0.4 to about 0.45, about 0.5 to about 0.7, about 0.45 to about 0.55, about 0.45 to about 0.65, about 0.55 to about 0.65, about 0.5 to about 0.55, about 0.6 to about 0.65 or about 0.7 to about 0.75. In certain embodiments, the isothermal amplification process is performed in the presence of a trehalose to PEG wt % ratio and/or uses a composition that includes trehalose and PEG at a wt % ratio of about 0.5 to about 0.7. In certain embodiments, the isothermal amplification process is performed in the presence of a trehalose to PEG wt % ratio and/or uses a composition that includes trehalose and PEG at a wt % ratio of about 0.45 to about 0.75. In certain embodiments, the isothermal amplification process is performed in the presence of a trehalose to PEG wt % ratio and/or uses a composition that includes trehalose and PEG at a wt % ratio of about 0.5 to about 0.75. In certain embodiments, the isothermal amplification process is performed in the presence of a trehalose to PEG wt % ratio and/or uses a composition that includes trehalose and PEG at a wt % ratio of about 0.45 to about 0.65. In certain embodiments, the isothermal amplification process is performed in the presence of a trehalose to PEG wt % ratio and/or uses a composition that includes trehalose and PEG at a wt % ratio of about 0.5 to about 0.65. In certain embodiments, the isothermal amplification process is performed in the presence of a trehalose to PEG wt % ratio and/or uses a composition that includes trehalose and PEG at a wt % ratio of about 0.5 to about 0.55. In certain embodiments, the isothermal amplification process is performed in the presence of a trehalose to PEG wt % ratio and/or uses a composition that includes trehalose and PEG at a wt % ratio of about 0.55 to about 0.65. In certain embodiments, the isothermal amplification process is performed in the presence of a trehalose to PEG wt % ratio and/or uses a composition that includes trehalose and PEG at a wt % ratio of about 0.6 to about 0.65. In certain embodiments, the isothermal amplification process is performed in the presence of a trehalose to PEG wt % ratio and/or uses a composition that includes trehalose and PEG at a wt % ratio of about 0.7 to about 0.75. In certain embodiments, the isothermal amplification process is performed in the presence of a trehalose to PEG wt % ratio and/or uses a composition that includes trehalose and PEG at a wt % ratio of about 0.3 to about 0.65. In certain embodiments, the isothermal amplification process is performed in the presence of a trehalose to PEG wt % ratio and/or uses a composition that includes trehalose and PEG at a wt % ratio of about 0.3 to about 0.60. In certain embodiments, the isothermal amplification process is performed in the presence of a trehalose to PEG wt % ratio and/or uses a composition that includes trehalose and PEG at a wt % ratio of about 0.3 to about 0.45.

In certain embodiments, the isothermal amplification process uses a composition that includes trehalose and PEG at a wt % ratio of about 0.3 to about 0.75.

In certain embodiments, the isothermal amplification process is performed in the presence of a trehalose to PEG wt % ratio and/or uses a composition that includes trehalose and PEG at a wt % ratio of about 0.79 or less, about 0.78 or less, about 0.77 or less, about 0.76 or less, about 0.75 or less, about 0.74 or less, about 0.73 or less, about 0.72 or less, about 0.71 or less, about 0.7 or less, about 0.69 or less, about 0.68 or less, about 0.67 or less, about 0.66 or less, about 0.65 or less, about 0.64 or less, about 0.63 or less, about 0.62 or less, about 0.61 or less, about 0.60 or less, about 0.59 or less, about 0.58 or less, about 0.57 or less, about 0.56 or less, about 0.55 or less, about 0.54 or less, about 0.53 or less, about 0.52 or less, about 0.51 or less, about 0.50 or less, about 0.49 or less, about 0.48 or less, about 0.47 or less, about 0.46 or less, about 0.45 or less, about 0.44 or less, about 0.43 or less, about 0.42 or less, about 0.41 or less, about 0.40 or less, about 0.39 or less, about 0.38 or less, about 0.37 or less, about 0.36 or less, about 0.35 or less, about 0.34 or less, about 0.33 or less, about 0.32 or less, about 0.31 or less, or about 0.3 or less. In certain embodiments, the isothermal amplification process is performed in the presence of a trehalose to PEG wt % ratio and/or uses a composition that includes trehalose and PEG at a wt % ratio of about 0.79 or less. In certain embodiments, the isothermal amplification process is performed in the presence of a trehalose to PEG wt % ratio and/or uses a composition that includes trehalose and PEG at a wt % ratio of about 0.75 or less. In certain embodiments, the isothermal amplification process is performed in the presence of a trehalose to PEG wt % ratio and/or uses a composition that includes trehalose and PEG at a wt % ratio of about 0.7 or less. In certain embodiments, the isothermal amplification process is performed in the presence of a trehalose to PEG wt % ratio and/or uses a composition that includes trehalose and PEG at a wt % ratio of about 0.65 or less. In certain embodiments, the isothermal amplification process is performed in the presence of a trehalose to PEG wt % ratio and/or uses a composition that includes trehalose and PEG at a wt % ratio of about 0.60 or less. In certain embodiments, the isothermal amplification process is performed in the presence of a trehalose to PEG wt % ratio and/or uses a composition that includes trehalose and PEG at a wt % ratio of about 0.55 or less. In certain embodiments, the isothermal amplification process is performed in the presence of a trehalose to PEG wt % ratio and/or uses a composition that includes trehalose and PEG at a wt % ratio of about 0.50 or less. In certain embodiments, the isothermal amplification process is performed in the presence of a trehalose to PEG wt % ratio and/or uses a composition that includes trehalose and PEG at a wt % ratio of about 0.45 or less. In certain embodiments, the isothermal amplification process is performed in the presence of a trehalose to PEG wt % ratio and/or uses a composition that includes trehalose and PEG at a wt % ratio of about 0.4 or less. In certain embodiments, the isothermal amplification process is performed in the presence of a trehalose to PEG wt % ratio and/or uses a composition that includes trehalose and PEG at a wt % ratio of about 0.35 or less. In certain embodiments, the isothermal amplification process is performed in the presence of a trehalose to PEG wt % ratio and/or uses a composition that includes trehalose and PEG at a wt % ratio of about 0.3 or less.

In certain embodiments, the isothermal amplification process is performed in the presence of a trehalose to PEG wt % ratio and/or uses a composition that includes a trehalose to PEG wt % ratio of no greater than about 0.79, no greater than about 0.78, no greater than about 0.77, no greater than about 0.76, no greater than about 0.75, no greater than about 0.74, no greater than about 0.73, no greater than about 0.72, no greater than about 0.71, no greater than about 0.7, no greater than about 0.69, no greater than about 0.68, no greater than about 0.67, no greater than about 0.66, no greater than about 0.65, no greater than about 0.64, no greater than about 0.63, no greater than about 0.62, no greater than about 0.61, no greater than about 0.60, no greater than about 0.59, no greater than about 0.58, no greater than about 0.57, no greater than about 0.56, no greater than about 0.55, no greater than about 0.56, no greater than about 0.55, no greater than about 0.54, no greater than about 0.53, no greater than about 0.52, no greater than about 0.51, no greater than about 0.50, no greater than about 0.49, no greater than about 0.48, no greater than about 0.47, no greater than about 0.46, no greater than about 0.45, no greater than about 0.44, no greater than about 0.43, no greater than about 0.42, no greater than about 0.41, no greater than about 0.40, no greater than about 0.39, no greater than about 0.38, no greater than about 0.37, no greater than about 0.36, no greater than about 0.35, no greater than about 0.34, no greater than about 0.33, no greater than about 0.32, no greater than about 0.31 or no greater than about 0.30. In certain embodiments, the isothermal amplification process is performed in the presence of a trehalose to PEG wt % ratio and/or uses a composition that includes a trehalose to PEG wt % ratio of no greater than about 0.79. In certain embodiments, the isothermal amplification process is performed in the presence of a trehalose to PEG wt % ratio and/or uses a composition that includes a trehalose to PEG wt % ratio of no greater than about 0.75. In certain embodiments, the isothermal amplification process is performed in the presence of a trehalose to PEG wt % ratio and/or uses a composition that includes a trehalose to PEG wt % ratio of no greater than about 0.73. In certain embodiments, the isothermal amplification process is performed in the presence of a trehalose to PEG wt % ratio and/or uses a composition that includes a trehalose to PEG wt % ratio of no greater than about 0.72. In certain embodiments, the isothermal amplification process is performed in the presence of a trehalose to PEG wt % ratio and/or uses a composition that includes a trehalose to PEG wt % ratio of no greater than about 0.7. In certain embodiments, the isothermal amplification process is performed in the presence of a trehalose to PEG wt % ratio and/or uses a composition that includes a trehalose to PEG wt % ratio of no greater than about 0.65. In certain embodiments, the isothermal amplification process is performed in the presence of a trehalose to PEG wt % ratio and/or uses a composition that includes a trehalose to PEG wt % ratio of no greater than about 0.62. In certain embodiments, the isothermal amplification process is performed in the presence of a trehalose to PEG wt % ratio and/or uses a composition that includes a trehalose to PEG wt % ratio of no greater than about 0.60. In certain embodiments, the isothermal amplification process is performed in the presence of a trehalose to PEG wt % ratio and/or uses a composition that includes a trehalose to PEG wt % ratio of no greater than about 0.55. In certain embodiments, the isothermal amplification process is performed in the presence of a trehalose to PEG wt % ratio and/or uses a composition that includes a trehalose to PEG wt % ratio of no greater than about 0.52. In certain embodiments, the isothermal amplification process is performed in the presence of a trehalose to PEG wt % ratio and/or uses a composition that includes a trehalose to PEG wt % ratio of no greater than about 0.45. In certain embodiments, the isothermal amplification process is performed in the presence of a trehalose to PEG wt % ratio and/or uses a composition that includes a trehalose to PEG wt % ratio of no greater than about 0.42. In certain embodiments, the isothermal amplification process is performed in the presence of a trehalose to PEG wt % ratio and/or uses a composition that includes a trehalose to PEG wt % ratio of no greater than about 0.35. In certain embodiments, the isothermal amplification process is performed in the presence of a trehalose to PEG wt % ratio and/or uses a composition that includes a trehalose to PEG wt % ratio of no greater than about 0.31.

In certain embodiments, the PEG for use in an isothermal amplification process of present disclosure has a molecular weight between about 15,000 to about 50,000 daltons. In certain embodiments, the PEG has a molecular weight between about 20,000 Daltons to about 50,000 daltons. In certain embodiments, the PEG has a molecular weight between about 30,000 Daltons to about 40,000 daltons, e.g., about 35,000 daltons. In certain embodiments, the PEG is PEG1450, PEG3000, PEG8000, PEG10000, PEG14000, PEG15000, PEG20000, PEG250000, PEG30000, PEG35000 or PEG40000. In certain embodiments, the PEG is PEG35000.

In certain embodiments, the PEG is present in an isothermal amplification process of the present disclosure, e.g., in a reagent mixture and/or solution, at an amount of about 2% to about 9% weight to volume (w/v). In certain embodiments, the PEG is present in an isothermal amplification process of the present disclosure, e.g., in a reagent mixture or solution, at an amount of about 2% to about 9% weight to volume (w/v). In certain embodiments, the PEG is present in an isothermal amplification process of the present disclosure, e.g., in a reagent mixture or solution, at an amount of about 2% to about 8.5% weight to volume (w/v). For example, but not by way of limitation, the PEG can be present in an amount of about 2.2% to about 8.5% w/v, about 2.4% to about 8.5% w/v, about 2.6% to about 8.5% w/v, about 2.8% to about 8.5% w/v, about 3% to about 8.5% w/v, about 3.2% to about 8.5% w/v, about 3.4% to about 8.5% w/v, about 3.6% to about 8.5% w/v, about 3.8% to about 8.5% w/v, about 4% to about 8.5% w/v, about 4.2% to about 8.5% w/v, about 4.4% to about 8.5% w/v, about 4.6% to about 8.5% w/v, about 4.8% to about 8.5% w/v, about 5% to about 8.5% w/v, about 5.2% to about 8.5% w/v, about 5.4% to about 8.5% w/v, about 5.6% to about 8.5% w/v, about 5.8% to about 8.5% w/v, about 6% to about 8.5% w/v, about 6.2% to about 8.5% w/v, about 6.4% to about 8.5% w/v, about 6.5% to about 8.5% w/v, about 6.6% to about 8.5% w/v, about 6.8% to about 8.5% w/v, about 7% to about 8.5% w/v, about 7.2% to about 8.5% w/v, about 7.4% to about 8.5% w/v, about 7.6% to about 8.5% w/v, about 7.8% to about 8.5% w/v, about 2% to about 7.8% w/v, about 2% to about 7.6% w/v, about 2% to about 7.4% w/v, about 2% to about 7.2% w/v, about 2% to about 7% w/v, about 2% to about 6.8% w/v, about 2% to about 6.6% w/v, about 2% to about 6.4% w/v, about 2% to about 6.2% w/v, about 2% to about 6% w/v, about 2% to about 5.8% w/v, about 2% to about 5.6% w/v, about 2% to about 5.4% w/v, about 2% to about 5.2% w/v, about 2% to about 5% w/v, about 2% to about 4.8% w/v, about 2% to about 4.6% w/v, about 2% to about 4.4% w/v, about 2% to about 4.2% w/v, about 2% to about 4% w/v, about 2% to about 3.8% w/v, about 2% to about 3.6% w/v, about 2% to about 3.4% w/v, about 2% to about 3.2% w/v, about 2% to about 3% w/v, about 2% to about 2.8% w/v, about 2% to about 2.6% w/v, about 2% to about 2.4% w/v, about 2% to about 2.2% w/v, about 3% to about 5% w/v, about 3% to about 7% w/v, about 4% to about 6% w/v, about 4% to about 7% w/v or about 4% to about 7% w/v. In certain embodiments, the PEG is present in the isothermal amplification process, e.g., in a reagent mixture or solution, in an amount of about 4% to about 9% w/v. In certain embodiments, the PEG is present in the isothermal amplification process in an amount of about 4% to about 6% w/v. In certain embodiments, the PEG is present in the isothermal amplification process in an amount of about 5% to about 7% w/v. In certain embodiments, the PEG is present in the isothermal amplification process in an amount of about 6% to about 7% w/v. In certain embodiments, the PEG is present in the isothermal amplification process in an amount of about 5% to about 8% w/v. In certain embodiments, the PEG is present in the isothermal amplification process in an amount of about 5% to about 9% w/v. In certain embodiments, the PEG is present in the isothermal amplification process in an amount of about 6% to about 8% w/v. In certain embodiments, the PEG is present in the isothermal amplification process in an amount of about 7% to about 8% w/v. In certain embodiments, the PEG is present in the isothermal amplification process in an amount of about 8% to about 9% w/v. In certain embodiments, the PEG is present in the isothermal amplification process in an amount of about 5.5% w/v. In certain embodiments, the PEG is present in the isothermal amplification process in an amount of about 6.3% w/v. In certain embodiments, the PEG is present in the isothermal amplification process in an amount of about 6.5% w/v. In certain embodiments, the PEG is present in the isothermal amplification process in an amount of about 7% w/v. In certain embodiments, the PEG is present in the isothermal amplification process in an amount of about 7.4% w/v. In certain embodiments, the PEG is present in the isothermal amplification process in an amount of about 7.7% w/v. In certain embodiments, the PEG is present in the isothermal amplification process in an amount of 8.25% w/v. In certain embodiments, the PEG is present in the isothermal amplification process in an amount of about 8.4% w/v. In certain embodiments, the PEG is present in the isothermal amplification process in an amount of about 8.5% w/v.

In certain embodiments, the trehalose is present in an isothermal amplification process of the present disclosure, e.g., in a reagent mixture or solution, at an amount of about 1% to about 12% w/v. In certain embodiments, the trehalose is present in an isothermal amplification process of the present disclosure, e.g., in a reagent mixture or solution, at an amount of about 2% to about 12% w/v. In certain embodiments, the trehalose is present in an isothermal amplification process of the present disclosure, e.g., in a reagent mixture or solution, at an amount of about 5% to about 12% w/v. In certain embodiments, the trehalose is present in an isothermal amplification process of the present disclosure, e.g., in a reagent mixture or solution, at an amount of about 2% to about 9% w/v. In certain embodiments, the trehalose is present in an isothermal amplification process of the present disclosure, e.g., in a reagent mixture or solution, at an amount of about 5% to about 9% w/v. For example, but not by way of limitation, the trehalose is present in the isothermal amplification process in an amount of about 1% to about 9% w/v, about 1.5% to about 9% w/v, about 1.8% to about 9% w/v, about 1.9% to about 9% w/v, 2.2% to about 9% w/v, about 2.4% to about 9% w/v, about 2.6% to about 9% w/v, about 2.8% to about 9% w/v, about 3% to about 9% w/v, about 3.2% to about 9% w/v, about 3.4% to about 9% w/v, about 3.6% to about 9% w/v, about 3.8% to about 9% w/v, about 4% to about 9% w/v, about 4.2% to about 9% w/v, about 4.4% to about 9% w/v, about 4.6% to about 9% w/v, about 4.8% to about 9% w/v, about 5% to about 9% w/v, about 5.2% to about 9% w/v, about 5.4% to about 9% w/v, about 5.6% to about 9% w/v, about 5.8% to about 9% w/v, about 6% to about 9% w/v, about 6.2% to about 9% w/v, about 6.4% to about 9% w/v, about 6.5% to about 9% w/v, about 6.6% to about 9% w/v, about 6.8% to about 9% w/v, about 7% to about 9% w/v, about 7.2% to about 9% w/v, about 7.4% to about 9% w/v, about 7.6% to about 9% w/v, about 7.8% to about 9% w/v, about 8% to about 9% w/v, about 8.2% to about 9% w/v, about 8.4% to about 9% w/v, about 8.6% to about 9% w/v, about 8.8% to about 9% w/v, 2% to about 8.8% w/v, about 2% to about 8.6% w/v, about 2% to about 8.4% w/v, about 2% to about 8.2% w/v, about 2.2% to about 8% w/v, about 2% to about 7.8% w/v, about 2% to about 7.6% w/v, about 2% to about 7.4% w/v, about 2% to about 7.2% w/v, about 2% to about 7% w/v, about 2% to about 6.8% w/v, about 2% to about 6.6% w/v, about 2% to about 6.4% w/v, about 2% to about 6.2% w/v, about 2% to about 6% w/v, about 2% to about 5.8% w/v, about 2% to about 5.6% w/v, about 2% to about 5.4% w/v, about 2% to about 5.2% w/v, about 2% to about 5% w/v, about 2% to about 4.8% w/v, about 2% to about 4.6% w/v, about 2% to about 4.4% w/v, about 2% to about 4.2% w/v, about 2% to about 4% w/v, about 2% to about 3.8% w/v, about 2% to about 3.6% w/v, about 2% to about 3.4% w/v, about 2% to about 3.2% w/v, about 2% to about 3% w/v, about 2% to about 2.8% w/v, about 2% to about 2.6% w/v, about 2% to about 2.4% w/v, about 2% to about 2.2% w/v, about 3% to about 7% w/v, about 3% to about 6% w/v, about 3% to about 5% w/v, about 4% to about 8% w/v, about 1% to about 2% w/v, about 1.5% to about 2% w/v, about 1.8% to about 2% w/v, about 1.9% to about 2% w/v. In certain embodiments, the trehalose is present in the isothermal amplification process in an amount of about 3% to about 12% w/v. In certain embodiments, the trehalose is present in the isothermal amplification process in an amount of about 7% to about 12% w/v. In certain embodiments, the trehalose is present in the isothermal amplification process in an amount of about 3% to about 8% w/v. In certain embodiments, the trehalose is present in the isothermal amplification process in an amount of about 3% to about 7% w/v. In certain embodiments, the trehalose is present in the isothermal amplification process in an amount of about 3% to about 6% w/v. In certain embodiments, the trehalose is present in the isothermal amplification process in an amount of about 3% to about 5% w/v. In certain embodiments, the trehalose is present in the isothermal amplification process in an amount of about 4% w/v. In certain embodiments, the trehalose is present in the isothermal amplification process in an amount of about 8% w/v. In certain embodiments, the trehalose is present in the isothermal amplification process in an amount of about 9% w/v. In certain embodiments, the trehalose is present in the isothermal amplification process in an amount of about 11% w/v. In certain embodiments, the trehalose is present in the isothermal amplification process in an amount of about 12% w/v.

### A. RPA Process

In certain embodiments, the isothermal amplification process is RPA. RPA relies on the properties of recombinase and related protein components to invade double-stranded nucleic acids with single stranded homologous nucleic acids permitting sequence specific priming of nucleic acid polymerase reactions.

RPA amplification reactions exploit enzymes known as recombinases, which form complexes with oligonucleotide primers and pair the primers with their homologous sequences in duplex nucleic acids. A single-stranded nucleic acid binding (SSB) protein binds to the displaced nucleic acid strand and stabilizes the resulting loop. Nucleic acid amplification is then initiated from the primer, but only if the target sequence is present. Once initiated, the amplification reaction progresses rapidly, so that starting with just a few target copies of nucleic acid, the highly specific amplification reaches detectable levels within minutes.

In certain embodiments, in a first step, a first and a second single stranded nucleic acid primer is contacted with a recombinase (e.g., UvsX), a recombinase loading agent (e.g., UvsY) and a single strand DNA binding protein (e.g., gp32) to form a first and a second nucleoprotein primer. The single stranded nucleic acid primers are specific for and are complementary to the target nucleic acid molecule. In the second step, the first nucleoprotein primer is contacted to the double stranded target nucleic acid molecule to create a first D loop structure at a first portion of the double stranded target nucleic acid molecule (Step 2a). Further, the second nucleoprotein primer is contacted to the double stranded target nucleic acid molecule to create a second D loop structure at a second portion of the double stranded target nucleic acid molecule (Step 2b). The D loop structures are formed such that the 3′ ends of the first nucleic acid primer and said second nucleic acid primer are oriented toward each other on the same double stranded target nucleic acid molecule without completely denaturing the target nucleic acid molecule. It should be noted that Step 2a and Step 2b can be performed in any order or simultaneously.

In a D loop structure, the primer is hybridized to one strand of the double stranded target nucleic acid molecule to form a double stranded structure. The second strand of the target nucleic acid molecule is displaced by the primer. The structure resembles a capital D where the straight part of the D represents the double stranded part of the structure and the curved part of the D represents the single stranded displaced second strand of the target nucleic acid.

In the third step, the 3′ end of the first and the second nucleoprotein primer is extended with one or more polymerases capable of strand displacement synthesis and dNTPs to generate a first and second double stranded target nucleic acid molecule and a first and second displaced strand of nucleic acid. The first and second double stranded target nucleic acid molecules may serve as target nucleic acid molecules in step two during subsequent rounds of amplification.

Steps two and step three are repeated until a desired degree of amplification of the target nucleic acid is achieved.

During the amplification process described above, the first and second displaced strand of nucleic acid may hybridize to each other after Step (c) to form a third double stranded target nucleic acid molecule.

In certain embodiments, the present disclosure provides an RPA process that includes trehalose and PEG at the ratios and amounts described herein, e.g., the ratios and amounts disclosed above. In certain embodiments, the present disclosure provides an RPA process that is performed in the presence of and/or uses a composition that includes a trehalose to PEG wt % ratio of about 0.25 to about 0.79. In certain embodiments, the present disclosure provides an RPA process that is performed in the presence of and/or uses a composition that includes a trehalose to PEG wt % ratio of about 0.3 to about 0.79. In certain embodiments, the present disclosure provides an RPA process that is performed in the presence of and/or uses a composition that includes a trehalose to PEG wt % ratio of about 0.3 to about 0.5. In certain embodiments, the present disclosure provides an RPA process that is performed in the presence of and/or uses a composition that includes a trehalose to PEG wt % ratio of about 0.3 to about 0.4. In certain embodiments, the present disclosure provides an RPA process that is performed in the presence of and/or uses a composition that includes a trehalose to PEG wt % ratio of about 0.4 to about 0.79. For example, but not by way of limitation, the present disclosure provides an RPA process that is performed in the presence of and/or uses a composition that includes a trehalose to PEG wt % ratio of about 0.45 to about 0.75, e.g., about 0.5 to about 0.7. In certain embodiments, the present disclosure provides an RPA process that is performed in the presence of and/or uses a composition that includes a trehalose to PEG wt % ratio of about 0.4 to about 0.45. In certain embodiments, the present disclosure provides an RPA process that is performed in the presence of and/or uses a composition that includes a trehalose to PEG wt % ratio of about 0.79 or less. In certain embodiments, the present disclosure provides an RPA process that is performed in the presence of and/or uses a composition that includes a trehalose to PEG wt % ratio of about 0.75 or less, e.g., 0.65 or less or 0.55 or less. In certain embodiments, the present disclosure provides an RPA process that is performed in the presence of and/or uses a composition that includes a trehalose to PEG wt % ratio of about 0.65 or less. In certain embodiments, the present disclosure provides an RPA process that is performed in the presence of and/or uses a composition that includes a trehalose to PEG wt % ratio of about 0.55 or less. In certain embodiments, the present disclosure provides an RPA process that is performed in the presence of and/or uses a composition that includes a trehalose to PEG wt % ratio of about 0.55 or less.

In certain embodiments, the present disclosure provides an RPA process that is performed in the presence of and/or uses a composition that includes about 2% to about 9% w/v of PEG.

In certain embodiments, the present disclosure provides an RPA process that is performed in the presence of about 5% to about 9% w/v of PEG, e.g., about 6% to about 9% w/v of PEG.

In certain embodiments, the present disclosure provides an RPA process that is performed in the presence of and/or uses a composition that includes about 2% to about 8% w/v of PEG, e.g., about 5% to about 8% w/v of PEG.

In certain embodiments, the present disclosure provides an RPA process that is performed in the presence of about 2% to about 8% w/v of PEG, e.g., about 5% to about 8% w/v of PEG.

In certain embodiments, the present disclosure provides an RPA process that is performed in the presence of and/or uses a composition that includes about 1% to about 12% w/v of trehalose, e.g., about 2% to about 12% w/v of trehalose. In certain embodiments, the present disclosure provides an RPA process that is performed in the presence of and/or uses a composition that includes about 2% to about 12% w/v of trehalose, e.g., about 3% to about 12% w/v of trehalose. In certain embodiments, the present disclosure provides an RPA process that is performed in the presence of and/or uses a composition that includes about 5% to about 12% w/v of trehalose, e.g., about 6% to about 12% w/v of trehalose. In certain embodiments, the present disclosure provides an RPA process that is performed in the presence of and/or uses a composition that includes about 3% to about 5% w/v of trehalose, e.g., about 4% w/v of trehalose.

In certain embodiments, the present disclosure provides an RPA process that is performed in the presence of about 5% to about 12% w/v of trehalose, e.g., about 6% to about 12% w/v of trehalose.

In certain embodiments, the present disclosure provides an RPA process that is performed in the presence of (i) about 5% to about 12% w/v of trehalose, e.g., about 6% to about 12% w/v of trehalose, and (ii) about 2% to about 8% w/v of PEG, e.g., about 5% to about 8% w/v of PEG.

In certain embodiments, the present disclosure provides an RPA process that is performed in the presence of (i) about 10% to about 12% w/v of trehalose, and (ii) about 2% to about 8% w/v of PEG, e.g., about 5% to about 8% w/v of PEG.

In certain embodiments, the RPA process further includes the use of a recombinase, a single-stranded binding protein, a polymerase, dNTPs, ATP, a primer and a template nucleic acid (e.g., target nucleic acid). In certain embodiments, an RPA process can include the use of one or more of the following (in any combination): at least one recombinase, at least one single-stranded DNA binding protein, at least one DNA polymerase, dNTPs, a buffer, a reducing agent, ATP or an ATP analog, at least one recombinase loading protein, a first primer and, optionally, a second primer, a probe, a reverse transcriptase and a template nucleic acid molecule, e.g., a single-stranded (e.g., RNA) or double stranded target nucleic acid. In certain embodiments, the RPA reaction can contain, e.g., a reverse transcriptase. In certain embodiments, the RPA reaction does not include a reverse transcriptase.

In certain embodiments, an RPA process of the present disclosure includes combining non-protein components (NPC) (e.g., a reaction buffer, dNTPs, ATP and salts), protein components (PC) that include the enzymes required for the RPA process (e.g., a DNA polymerase, a recombinase, a recombinase loading protein, a single stranded binding protein, creatine kinase, a nuclease (e.g., an exonuclease) and/or a reverse transcriptase) and oligonucleotide components (OC) (e.g., one or more primers and/or one or more probes specific to a target nucleic acid or two or more target nucleic acids (e.g., in a multiplex RPA process)) with the sample containing the target nucleic acid(s). In certain embodiments, these components can be added in any order to the sample, or the components can be included in a “master mix” that is then added to the sample. In certain embodiments, the trehalose and PEG at the ratios and/or amounts described herein are present in the NPC. In certain embodiments, the trehalose and PEG at the ratios described herein are present in the NPC. Alternatively, the trehalose and PEG at the ratios and/or amounts described herein are present in the master mix. In certain embodiments, the trehalose and PEG at the amounts described herein are present in the master mix. In certain embodiments, an activator, e.g., magnesium (e.g., as magnesium acetate (MgOAc)), is then added to the reaction, followed by the incubation of the reaction at a specified temperature, e.g., 40 °C, to amplify the target nucleic acid(s).

In certain embodiments, a portion of the NPC and/or master mix is contacted with the sample to amplify the target nucleic acid(s) present in the sample. For example, but not by way of limitation, a portion of the NPC and/or master mix that includes the trehalose and PEG at the ratios and/or amounts described herein is contacted with the sample to amplify the target nucleic acid(s) present in the sample. For example, but not by way of limitation, about 5%, about 10%, about 15%, about 20%, about 25%, about 30%, about 35%, about 40%, about 45%, about 50%, about 55%, about 60%, about 65%, about 70% or about 75% of a composition (e.g., an NPC composition) of the present disclosure is added to an isothermal amplification process. In certain embodiments, about 5% or less, about 10% or less, about 15% or less, about 20% or less, about 25% or less, about 30% or less, about 35% or less, about 40% or less, about 45% or less, about 50% or less, about 55% or less, about 60% or less, about 65% or less, about 70% or less or about 75% or less of a composition (e.g., an NPC composition) of the present disclosure is added to an isothermal amplification process.

In certain embodiments, the recombinase (e.g., UvsX), recombinase loading agent (e.g., UvsY) and single strand DNA binding protein (e.g., gp32) can be derived from a myoviridae phage. In certain embodiments, the myoviridae phage can be, for example, T4, T2, T6, Rb69, Aeh1, KVP40, Acinetobacter phage 133, Aeromonas phage 65, cyanophage P-SSM2, cyanophage PSSM4, cyanophage S-PM2, Rb14, Rb32, Aeromonas phage 25, Vibrio phage nt-1, phi-1, Rb16, Rb43, Phage 31, phage 44RR2.8t, Rb49, phage Rb3 or phage LZ2. In certain embodiments, the combination of Rb69 UvsX, Rb69 UvsY and Rb69 gp32 can be used. In certain embodiments, the combination of Aeh1 UvsX, Aeh1 UvsY and Rb69 gp32 can be used. In certain embodiments, the combination of T4 UvsX, T4 UvsY and Rb69 gp32 can be used. In certain embodiments, the combination of T4 UvsX, Rb69 UvsY and T4 gp32 can be used.

In certain embodiments, the recombinase (e.g., UvsX), recombinase loading agent (e.g., UvsY) and single strand DNA binding protein (e.g., gp32) can each be native, hybrid or mutant proteins from the same or different myoviridae phage sources. A native protein can be a wildtype or natural variant of a protein. A mutant protein (also called a genetically engineered protein) is a native protein with natural or manmade mutations such as insertions, deletions, substitutions, or a combination thereof, that are at the N terminus, C terminus, or interior (between the N terminus and the C terminus). A hybrid protein (also called a chimeric protein) comprises sequences from at least two different organisms. For example, but not by way of limitation, a hybrid UvsX protein can contain an amino acid from one species (e.g., T4) but a DNA binding loop from another species (e.g., T6). The hybrid protein can contain improved characteristics compared to a native protein. The improved characteristics can be increased or more rapid RPA amplification rate or a decreased or more controllable RPA amplification rate.

In certain embodiments, the recombinase (e.g., UvsX) can be a mutant UvsX. In certain embodiments, the mutant UvsX is an Rb69 UvsX comprising at least one mutation in the Rb69 UvsX amino acid sequence, wherein the mutation is selected from the group consisting of (a) an amino acid which is not histidine at position 64, a serine at position 64, the addition of one or more glutamic acid residues at the C-terminus, the addition of one or more aspartic acid residues at the C-terminus, and a combination thereof. In certain embodiments, the mutant UvsX is a T6 UvsX having at least one mutation in the T6 UvsX amino acid sequence, wherein the mutation is selected from the group consisting of (a) an amino acid which is not histidine at position 66; (b) a serine at position 66; (c) the addition of one or more glutamic acid residues at the C-terminus; (d) the addition of one or more aspartic acid residues at the C-terminus; and (e) a combination thereof.

In certain embodiments, the RPA processes of the present disclosure are performed with a polymerase which is a large fragment polymerase. In certain embodiments, the large fragment polymerase can be selected from the group consisting of E. Coli Pol I, Bacillus subtilis Pol I, Staphylococcus aureus Pol I and homologs thereof. In certain embodiments, the RPA processes are performed in the presence of about 0.01 mg/mL to about 0.5 mg/mL of a DNA Polymerase, e.g., about 0.08 mg/mL to about 0.2 mg/ml of a DNA Polymerase. In certain embodiments, the RPA processes are performed in the presence of about 10 units/mL to about 10,000 units/mL of a DNA Polymerase, e.g., about 500 units/mL to about 5,000 units/mL of a DNA Polymerase.

In certain embodiments, the RPA processes are performed in the presence of heparin. Heparin can serve as an agent to reduce the level of non-specific primer noise, and to increase the ability of E. coli exonuclease III or E. coli exonuclease IV to rapidly polish 3′ blocking groups or terminal residues from recombination intermediates.

In certain embodiments, the dNTPs for use in the RPA processes of the present disclosure include, for example, dATP, dGTP, dCTP and dTTP. In certain embodiments, the ddNTPs for use in the RPA processes of the present disclosure include, for example, ddATP, ddTTP, ddGTP and ddGTP. In certain embodiments, dNTPs and/or ddNTPs can be used at concentrations of about 1 μM to about 500 μM per each dNTP and/or ddNTP species.

In certain embodiments, the RPA processes are performed with a blocked primer. A blocked primer is a primer which does not allow elongation with a polymerase. Where a blocked primer is used, an unblocking agent is also used to unblock the primer to allow elongation. The unblocking agent can be an endonuclease or exonuclease which can cleave the blocking group from the primer. In certain embodiments, unblocking agents include E. coli exonuclease III and E. coli endonuclease IV. In certain embodiments, the unblocking agent is E. coli exonuclease III. In certain embodiments, the unblocking agent is E. coli endonuclease IV.

In certain embodiments, the RPA processes are performed in the presence of two or more primers, e.g., (i) at least one or more forward primers, (ii) at least one or more reverse primers or (iii) at least one or more forward and reverse primers, and/or at least one or more probes. In certain embodiments, the RPA processes are performed in the presence of at least three primers. In certain embodiments, the RPA processes are performed in the presence of at least two probes. In certain embodiments, the RPA processes are performed in the presence of about 1 nM to about 1,000 nM of one or more primers and/or probes, e.g., about 10 nM to about 500 nM of one or more primers and/or probes. In certain embodiments, the RPA processes are performed in the presence of about 1 nM to about 1,000 nM of one or more primers, e.g., about 10 nM to about 500 nM of one or more primers. In certain embodiments, the RPA processes are performed in the presence of about 1 nM to about 1,000 nM of a forward primer, e.g., about 10 nM to about 500 nM of a forward primer. In certain embodiments, the RPA processes are performed in the presence of about 1 nM to about 1,000 nM of a reverse primer, e.g., about 10 nM to about 500 nM of a reverse primer. In certain embodiments, the RPA processes are performed in the presence of about 1 nM to about 1,000 nM of one or more probes, e.g., about 10 nM to about 500 nM of one or more probes, e.g., detection probes.

In certain embodiments, only one of the nucleic acid primers used in the RPA processes of the present disclosure is coated with recombinase/recombinase loading agent/single stranded DNA binding protein. That is, an RPA can be performed with one primer which is uncoated and one primer which is coated with any one or a combination of recombinase, recombinase loading agent and single stranded DNA binding protein.

In certain embodiments, the RPA processes are performed in the presence of about 1 mM to about 25 mM magnesium ions, e.g., about 1 mM to about 20 mM, about 1 mM to about 10 mM or about 1 mM to about 3 mM magnesium ions. In certain embodiments, the RPA processes are performed in the presence of about 1 mM to about 25 mM divalent manganese ions, e.g., about 1 mM to about 20 mM, about 1 mM to about 10 mM or about 1 mM to about 3 mM divalent manganese ions. In certain embodiments, the manganese ions replace the magnesium ions and the reaction can be performed with or without magnesium.

In certain embodiments, the recombinase loading agent (e.g., UvsY) is omitted. That is, any of the RPA reactions of this disclosure can be performed in the absence of the recombinase loading agent (e.g., UvsY).

In certain embodiments, an RPA process of the present disclosure can be employed using RNA as an initial template, e.g., to amplify a target nucleic acid derived from an RNA virus, by using reverse transcriptase to first produce a DNA copy of the RNA template after which the DNA copy can be subjected to RPA-based nucleic acid amplification. Performing RPA with RNA templates is typically referred to in the art as Reverse Transcriptase RPA or RT-RPA. In certain embodiments, the reverse transcriptase used in the methods of the present disclosure can be selected from: OmniScript (Qiagen), SensiScript (Qiagen), MonsterScript (Epicentre), Transcriptor (Roche), HIV RT (Ambion), Superscript III (Invitrogen), ThermoScript (Invitrogen), Thermo-X (Invitrogen), ImProm II (Promega) and EIAV-RT. In certain embodiments, the reverse transcriptase is EIAV-RT.

In certain embodiments, the reverse transcriptase can be omitted from the RPA reaction. For example, but not by way of limitation, any of the RPA reactions of the present disclosure can be performed in the absence of a reverse transcriptase. In certain embodiments, an RPA reaction of the present disclosure is performed in the absence of a reverse transcriptase if the target nucleic acid to be analyzed is DNA.

In certain embodiments, the ATP or analog thereof can be used at a concentration of about 1 and about 10 mM. Non-limiting examples of an ATP analog include ATP-γ-S, ATP-β-S and ddATP.

In certain embodiments, the following reagents, in addition to trehalose and PEG, can be employed for performing an RPA process of the present disclosure: a buffer (e.g., Tris-HCl), a reducing agent (e.g., DTT), Potassium Acetate, a second crowding agent, dNTPs, ATP, Phosphocreatine, Glycerol, Creatine Kinase, UvsX, UvsY, DNA polymerase, GP32, Exonuclease III, BSA, an activator (e.g., magnesium, e.g., Mg Acetate (MgOAc)) and EIAV. In certain embodiments, additional reagents can be employed, including but not limited to, forward primers, reverse primers, probes and ROX reference dyes.

In certain embodiments, about 5 mM to about 100 mM Tris-HCl at a pH of about 6.5-9.0, e.g., 8.3, can be employed in an RPA process of the present disclosure or included in a composition of the present disclosure.

In certain embodiments, about 5 mM to about 10 mM of a reducing agent (e.g., DTT) can be employed in an RPA process of the present disclosure or included in a composition of the present disclosure.

In certain embodiments, about 50 mM to about 100 mM potassium acetate can be employed in an RPA process of the present disclosure or included in a composition of the present disclosure.

In certain embodiments, about 1 mM to about 5 mM dNTPs can be employed in an RPA process of the present disclosure or included in a composition of the present disclosure.

In certain embodiments, about 1 mM to about 10 mM of ATP, e.g., about 2 mM to about 5 mM ATP, can be employed in an RPA process of the present disclosure or included in a composition of the present disclosure.

In certain embodiments, about 20 mM to about 100 mM Phosphocreatine, e.g., about 40 mM to about 100 mM Phosphocreatine, can be employed in an RPA process of the present disclosure or included in a composition of the present disclosure.

In certain embodiments, about 5 mM to about 40 mM Mg Acetate, e.g., about 10 mM to about 40 mM Mg Acetate, can be employed in an RPA process of the present disclosure or included in a composition of the present disclosure.

In certain embodiments, about 0.01 mg/mL to about 10 mg/mL BSA can be employed in an RPA process of the present disclosure or included in a composition of the present disclosure.

In certain embodiments, about 5% to about 10% Glycerol can be employed in an RPA process of the present disclosure or included in a composition of the present disclosure.

In certain embodiments, about 0.01 mg/mL to about 0.5 mg/mL Creatine Kinase, e.g., about 0.1 mg/mL to about 0.5 mg/mL Creatine Kinase, can be employed in an RPA process of the present disclosure or included in a composition of the present disclosure.

In certain embodiments, about 0.1 mg/mL to about 1.0 mg/mL UvsX, e.g., about 0.3 mg/mL to about 1.0 mg/mL UvsX, can be employed in an RPA process of the present disclosure or included in a composition of the present disclosure.

In certain embodiments, about 0.01 mg/mL to about 0.25 mg/mL UvsY, e.g., about 0.09 mg/mL to about 0.25 mg/mL UvsY, can be employed in an RPA process of the present disclosure or included in a composition of the present disclosure.

In certain embodiments, about 0.01 mg/mL to about 0.5 mg/mL DNA Polymerase, e.g., about 0.08 mg/mL to about 0.2 mg/mL DNA Polymerase, can be employed in an RPA process of the present disclosure or included in a composition of the present disclosure.

In certain embodiments, about 0.1 mg/mL to about 2.0 mg/mL GP32, e.g., about 0.4 mg/mL to about 0.8 mg/mL GP32, can be employed in an RPA process of the present disclosure or included in a composition of the present disclosure.

In certain embodiments, about 0.01 mg/mL to about 0.5 mg/mL Exonuclease III, e.g., about 0.1 mg/mL to about 0.5 mg/mL Exonuclease III, can be employed in an RPA process of the present disclosure or included in a composition of the present disclosure.

In certain embodiments, about 0.5 μg/mL to about 100.0 μg/mL of a reverse transcriptase (e.g., equine infectious anemia virus reverse transcriptase (EIAV-RT)), e.g., about 0.5 μg/mL to about 1.5 μg/mL, about 1.5 μg/mL to about 20 μg/mL or about 20 μg/mL to about 70 μg/mL EIAV-RT, can be employed in an RPA process of the present disclosure or included in a composition of the present disclosure.

In certain embodiments, the following reagents, in addition to trehalose and PEG, can be employed at the following concentrations (or included in a reagent composition of the present disclosure) for performing an RPA process of the present disclosure: about 5 mM to about 100 mM Tris-HCl at pH of about 6.5-9.0, e.g., 8.3; about 5 mM to about 10 mM of a reducing agent (e.g., DTT); about 50 mM to about 100 mM potassium acetate; about 1 mM to about 5 mM dNTPs; about 1 mM to about 10 mM of ATP, e.g., about 2 mM to about 5 mM ATP; about 20 mM to about 100 mM Phosphocreatine, e.g., about 40 mM to about 100 mM Phosphocreatine; about 5 mM to about 40 mM Mg Acetate, e.g., about 10 mM to about 40 mM Mg Acetate; about 0.01 mg/mL to about 10 mg/mL BSA; about 5% to about 10% Glycerol; about 0.01 mg/mL to about 0.5 mg/mL Creatine Kinase, e.g., about 0.1 mg/mL to about 0.5 mg/mL Creatine Kinase; about 0.1 mg/mL to about 1.0 mg/mL UvsX, e.g., about 0.3 mg/mL to about 1.0 mg/mL UvsX; about 0.01 mg/mL to about 0.25 mg/mL UvsY, e.g., about 0.09 mg/mL to about 0.25 mg/mL UvsY; about 0.01 mg/mL to about 0.5 mg/mL DNA Polymerase, e.g., about 0.08 mg/mL to about 0.2 mg/mL DNA Polymerase; about 0.1 mg/mL to about 2.0 mg/mL GP32, e.g., about 0.4 mg/mL to about 0.8 mg/mL GP32; about 0.01 mg/mL to about 0.5 mg/mL Exonuclease III, e.g., about 0.1 mg/mL to about 0.5 mg/mL Exonuclease III; and about 0.5 μg/mL to about 100.0 μg/mL equine infectious anemia virus reverse transcriptase (EIAV-RT), e.g., about 0.5 μg/mL to about 1.5 μg/mL EIAV-RT, about 1.5 μg/mL to about 20 μg/mL or about 20 μg/mL to about 70 μg/mL EIAV-RT. In certain embodiments, additional reagents can be employed, including but not limited to, forward primers, reverse primers, probes and ROX reference dyes.

In certain embodiments, the RPA process is performed in the presence of (i) about 5% to about 12% w/v of trehalose, e.g., about 6% to about 12% w/v of trehalose and (ii) about 2% to about 8% w/v of PEG, e.g., about 5% to about 8% w/v of PEG; and one or more of the following: about 5 mM to about 10 mM of a reducing agent (e.g., DTT); about 50 mM to about 100 mM potassium acetate; about 1 mM to about 5 mM dNTPs; about 1 mM to about 10 mM of ATP, e.g., about 2 mM to about 5 mM ATP; about 20 mM to about 100 mM Phosphocreatine, e.g., about 40 mM to about 100 mM Phosphocreatine; about 5 mM to about 40 mM Mg Acetate, e.g., about 10 mM to about 40 mM Mg Acetate; about 0.01 mg/mL to about 10 mg/mL BSA; about 5% to about 10% Glycerol; about 0.01 mg/mL to about 0.5 mg/mL Creatine Kinase, e.g., about 0.1 mg/mL to about 0.5 mg/mL Creatine Kinase; about 0.1 mg/mL to about 1.0 mg/mL UvsX, e.g., about 0.3 mg/mL to about 1.0 mg/mL UvsX; about 0.01 mg/mL to about 0.25 mg/mL UvsY, e.g., about 0.09 mg/mL to about 0.25 mg/mL UvsY; about 0.01 mg/mL to about 0.5 mg/mL DNA Polymerase, e.g., about 0.08 mg/mL to about 0.2 mg/mL DNA Polymerase; about 0.1 mg/mL to about 2.0 mg/mL GP32, e.g., about 0.4 mg/mL to about 0.8 mg/mL GP32; about 0.01 mg/mL to about 0.5 mg/mL Exonuclease III, e.g., about 0.1 mg/mL to about 0.5 mg/mL Exonuclease III; and about 0.5 μg/mL to about 100.0 g/mL equine infectious anemia virus reverse transcriptase (EIAV-RT), e.g., about 0.5 μg/mL to about 1.5 μg/mL EIAV-RT, about 1.5 μg/mL to about 20 μg/mL or about 20 μg/mL to about 70 μg/mL EIAV-RT.

In certain embodiments, the RPA process is performed in the presence of (i) about 5% to about 12% w/v of trehalose, e.g., about 6% to about 12% w/v of trehalose; (ii) about 2% to about 8% w/v of PEG, e.g., about 5% to about 8% w/v of PEG; (iii) about 20 mM to about 100 mM Phosphocreatine, e.g., about 40 mM to about 100 mM Phosphocreatine; (iv) about 5 mM to about 40 mM Mg Acetate, e.g., about 10 mM to about 40 mM Mg Acetate; (v) about 0.01 mg/mL to about 0.5 mg/mL Creatine Kinase, e.g., about 0.1 mg/mL to about 0.5 mg/mL Creatine Kinase; (vi) about 0.1 mg/mL to about 1.0 mg/mL UvsX, e.g., about 0.3 mg/mL to about 1.0 mg/mL UvsX; (vii) about 0.01 mg/mL to about 0.25 mg/mL UvsY, e.g., about 0.09 mg/mL to about 0.25 mg/mL UvsY; (viii) about 0.01 mg/mL to about 0.5 mg/mL DNA Polymerase, e.g., about 0.08 mg/mL to about 0.2 mg/mL DNA Polymerase; (ix) about 0.1 mg/mL to about 2.0 mg/mL GP32, e.g., about 0.4 mg/mL to about 0.8 mg/mL GP32; and (x) about 0.01 mg/mL to about 0.5 mg/mL Exonuclease III, e.g., about 0.1 mg/mL to about 0.5 mg/mL Exonuclease III.

In certain embodiments, a singleplex RPA reaction for amplifying and detecting a single target nucleic acid, e.g., from a pathogen or infectious agent (e.g., HIV-1, HIV-2, HCV or HBV), can comprise the following reagents, in addition to trehalose and PEG, employed at the following concentrations: Tris-HCl 1M, pH 8.3 at 50 mM, DTT at 5 mM, Potassium Acetate at 100 mM, 20% Polyethylene glycol at 5.5%, 100 mM dNTPs at 1.8 mM, ATP at 2.5 mM, Phosphocreatine at 50 mM, Forward Primer at 420 nM, Reverse Primer at 420 nM, Exo Probe at 120 nM, ROX reference dye at 15 nM, Glycerol at 6.5%, Creatine Kinase at 0.1 mg/ml, UvsX at 0.3 mg/ml, UvsY at 0.09 mg/ml, DNA Polymerase at 0.0798 mg/ml, Gp32 at 0.48 mg/ml, Exonuclease III at 0.1 mg/ml, Bovine Serum Albumin (BSA) at 0.02 mg/ml, EIAV Reverse Transcriptase at 0.0008 mg/ml and/or Magnesium Acetate at 14 mM.

In certain embodiments, a multiplex RPA reaction for amplifying and detecting at least two target nucleic acids, e.g., for detecting HIV-1 and HBV, can comprise the following reagents, in addition to trehalose and PEG, employed at the following concentrations: Tris-HCl 1M, pH 8.3 at 50 mM, DTT at 5 mM, Potassium Acetate at 100 mM, 20% Polyethylene glycol at 5.5%, 100 mM dNTPs at 2.7 mM, ATP at 3.5 mM, Phosphocreatine at 50 mM, HIV-1 INT Forward Primer at 157.50 nM, HIV-1 INT Reverse Primer at 236.37 nM, HIV-1 INT Exo Probe at 90 nM, HIV-1 LTR Forward Primer at 39.37 nM, HIV-1 LTR Reverse Primer at 39.37 nM, HIV-1 INT Exo Probe at 22.5 nM, HBV Forward Primer at 86.13 nM, HBV Reverse Primer at 86.13 nM, HBV Exo Probe at 90 nM, ROX reference dye at 45 nM, Glycerol at 8.4%, Creatine Kinase at 0.1 mg/ml, UvsX at 0.375 mg/ml, UvsY at 0.0675 mg/ml, DNA Polymerase at 0.1396 mg/ml, Gp32 at 1.2 mg/ml, Exonuclease III at 0.1 mg/ml, Bovine Serum Albumin (BSA) at 0.02 mg/ml, EIA V Reverse Transcriptase at 0.0016 mg/ml and/or Magnesium Acetate at 14 mM.

In certain embodiments, a multiplex reaction for amplifying and detecting at least two target nucleic acids, e.g., for detecting HIV-1 and HBV, can comprise the following reagents, in addition to trehalose and PEG, employed at the following concentrations: Tris-HCl 1M, pH 8.3 at 50 mM, DTT at 5 mM, Potassium Acetate at 100 mM, 20% Polyethylene glycol at 5.5%, 100 mM dNTPs at 2.7 mM, ATP at 3.5 mM, Phosphocreatine at 50 mM, HIV-1 Forward Primer at 157.50 nM, HIV-1 Reverse Primer at 236.37 nM, HIV-1 Exo Probe at 90 nM, HBV Forward Primer at 86.13 nM, HBV Reverse Primer at 86.13 nM, HBV Exo Probe at 90 nM, ROX reference dye at 45 nM, Glycerol at 8.4%, Creatine Kinase at 0.1 mg/ml, UvsX at 0.375 mg/ml, UvsY at 0.0675 mg/ml, DNA Polymerase at 0.1396 mg/ml, Gp32 at 1.2 mg/ml, Exonuclease III at 0.1 mg/ml, Bovine Serum Albumin (BSA) at 0.02 mg/ml, EIAV Reverse Transcriptase at 0.0016 mg/ml and/or Magnesium Acetate at 14 mM.

In certain embodiments, an RPA process of the present disclosure includes contacting a sample comprising the target nucleic acid with a reagent composition comprising about 5% to about 12% w/v of trehalose, e.g., about 6% to about 12% w/v of trehalose, and about 2% to about 8% w/v of PEG, e.g., about 5% to about 8% w/v of PEG. In certain embodiments, an RPA process of the present disclosure includes (a) contacting a sample comprising a plurality of nucleic acids with a reagent composition comprising (i) about 5% to about 12% w/v of trehalose, e.g., about 6% to about 12% w/v of trehalose, and (ii) about 2% to about 8% w/v of PEG, e.g., about 5% to about 8% w/v of PEG, to generate a reaction mixture and (b) incubating the reaction mixture to amplify the target nucleic acid if present in the plurality of nucleic acids. In certain embodiments, the reagent composition can further comprise one or more reagents disclosed herein. For example, but not by way of limitation, the reagent composition can further include (iii) about 20 mM to about 100 mM Phosphocreatine, e.g., about 40 mM to about 100 mM Phosphocreatine; (iv) about 5 mM to about 40 mM Mg Acetate, e.g., about 10 mM to about 40 mM Mg Acetate; (v) about 0.01 mg/mL to about 0.5 mg/mL Creatine Kinase, e.g., about 0.1 mg/mL to about 0.5 mg/ml Creatine Kinase; (vi) about 0.1 mg/mL to about 1.0 mg/mL UvsX, e.g., about 0.3 mg/mL to about 1.0 mg/mL UvsX; (vii) about 0.01 mg/mL to about 0.25 mg/mL UvsY, e.g., about 0.09 mg/mL to about 0.25 mg/mL UvsY; (viii) about 0.01 mg/mL to about 0.5 mg/mL DNA Polymerase, e.g., about 0.08 mg/mL to about 0.2 mg/mL DNA Polymerase; (ix) about 0.1 mg/mL to about 2.0 mg/mL GP32, e.g., about 0.4 mg/mL to about 0.8 mg/mL GP32; and (x) about 0.01 mg/mL to about 0.5 mg/mL Exonuclease III, e.g., about 0.1 mg/mL to about 0.5 mg/mL Exonuclease III.

In certain embodiments, an RPA process of the present disclosure includes contacting a sample comprising the target nucleic acid with a composition disclosed herein, e.g., a composition comprising trehalose to PEG at a wt % ratio of about 0.25 to about 0.79. In certain embodiments, an RPA process of the present disclosure includes contacting a sample comprising the target nucleic acid with a composition disclosed herein, e.g., a composition comprising trehalose to PEG at a wt % ratio of about 0.3 to about 0.79. In certain embodiments, an RPA process of the present disclosure includes contacting a sample comprising the target nucleic acid with a composition disclosed herein, e.g., a composition comprising trehalose to PEG at a wt % ratio of about 0.45 to about 0.75, e.g., a wt % ratio of about 0.5 to about 0.7. In certain embodiments, an RPA process of the present disclosure includes contacting a sample comprising the target nucleic acid with a composition disclosed herein, e.g., a composition comprising trehalose to PEG at a wt % ratio of about 0.3 to about 0.45. In certain embodiments, the present disclosure provides an RPA process that includes contacting a sample comprising the target nucleic acid with a composition disclosed herein, e.g., a composition comprising trehalose to PEG at a wt % ratio of about 0.79 or less. In certain embodiments, the present disclosure provides an RPA process that includes contacting a sample comprising the target nucleic acid with a composition disclosed herein, e.g., a composition comprising trehalose to PEG at a wt % ratio of about 0.75 or less, e.g., a wt % ratio of about 0.65 or less, about 0.55 or less, about 0.45 or less, or about 0.35 or less. In certain embodiments, an RPA process of the present disclosure for amplifying a target nucleic acid includes: (a) contacting a sample (e.g., solution) comprising a plurality of nucleic acids with a composition comprising trehalose to PEG at a wt % ratio of about 0.3 to about 0.79 (e.g., about 0.45 to about 0.75, e.g., about 0.5 to about 0.7, e.g., about 0.3 to about 0.45), to generate a reaction mixture and (b) incubating the reaction mixture to amplify the target nucleic acid if present in the plurality of nucleic acids. In certain embodiments, an RPA process of the present disclosure for amplifying a target nucleic acid includes: (a) contacting a composition comprising trehalose to PEG at a wt % ratio of about 0.3 to about 0.79 (e.g., about 0.45 to about 0.75, e.g., about 0.5 to about 0.7, e.g., about 0.3 to about 0.45) with at least one additional composition including one or more other RPA reagents followed by contacting the mixture with a sample (e.g., solution) comprising a plurality of nucleic acids to generate a reaction mixture and (b) incubating the reaction mixture to amplify the target nucleic acid if present in the plurality of nucleic acids.

In certain embodiments, about 5%, about 10%, about 15%, about 20%, about 25%, about 30%, about 35%, about 40%, about 45%, about 50%, about 55%, about 60%, about 65%, about 70% or about 75% of a composition of the present disclosure is added to the sample or to other RPA reagents to result in a PEG concentration of about 2% to about 9% w/v in the RPA process, e.g., about 5% to about 9% PEG or about 5% to about 8% PEG. In certain embodiments, about 5% or less, about 10% or less, about 15% or less, about 20% or less, about 25% or less, about 30% or less, about 35% or less, about 40% or less, about 45% or less, about 50% or less, about 55% or less, about 60% or less, about 65% or less, about 70% or less or about 75% or less of a composition of the present disclosure is added to the sample or to other RPA reagents to result in a PEG concentration of about 2% to about 9% w/v in the RPA process, e.g., about 5% to about 9% PEG or about 5% to about 8% PEG. In certain embodiments, the RPA reaction or the composition includes about 2% to about 8% w/v of PEG, e.g., about 5% to about 8% w/v of PEG.

In certain embodiments, about 5%, about 10%, about 15%, about 20%, about 25%, about 30%, about 35%, about 40%, about 45%, about 50%, about 55%, about 60%, about 65%, about 70% or about 75% of a composition of the present disclosure is added to the sample or to other RPA reagents to reduce the trehalose concentration from the composition to about 2% to about 6% w/v trehalose. In certain embodiments, about 5%, about 10%, about 15%, about 20%, about 25%, about 30%, about 35%, about 40%, about 45%, about 50%, about 55%, about 60%, about 65%, about 70% or about 75% of a composition of the present disclosure is added to the sample or to other RPA reagents to reduce the trehalose concentration from the composition to about 1.5% to about 6% w/v trehalose. In certain embodiments, about 5% or less, about 10% or less, about 15% or less, about 20% or less, about 25% or less, about 30% or less, about 35% or less, about 40% or less, about 45% or less, about 50% or less, about 55% or less, about 60% or less, about 65% or less, about 70% or less or about 75% or less of a composition of the present disclosure is added to the sample or to other RPA reagents to reduce the trehalose concentration from the composition to about 2% to about 6% w/v trehalose. In certain embodiments, about 5% or less, about 10% or less, about 15% or less, about 20% or less, about 25% or less, about 30% or less, about 35% or less, about 40% or less, about 45% or less, about 50% or less, about 55% or less, about 60% or less, about 65% or less, about 70% or less or about 75% or less of a composition of the present disclosure is added to the sample or to other RPA reagents to reduce the trehalose concentration from the composition to about 1.5% to about 6% w/v trehalose. In certain embodiments, the RPA reaction or the composition includes about 2% to about 6% w/v or about 2% to about 5% w/v of trehalose, e.g., about 2% w/v of trehalose or about 3% w/v of trehalose.

In certain embodiments, the RPA reaction or the composition includes about 3% to about 6% w/v or about 3% to about 5% w/v of trehalose, e.g., about 4% w/v of trehalose.

In certain embodiments, the composition can include one or more of RPA reagents disclosed herein, e.g., reagents required for performing an RPA process and/or support an RPA process. Non-limiting examples of such reagents include a recombinase, a single-stranded DNA binding protein, a recombinase loading protein, a creatine kinase, a nuclease, a reverse transcriptase a DNA polymerase, dNTPs, a buffer, a crowding agent, a reducing agent, ATP or an ATP analog, a first primer, a second primer and a probe.

In certain embodiments, a method of the present disclosure can further include detecting the presence and/or absence of the target nucleic acid. In certain embodiments, a method of the present disclosure can further include detecting the amount of the target nucleic acid.

In certain embodiments, the reaction volume of an RPA process of the present disclosure can be about 5 μl, about 10 μl, about 20 μl, about 30 μl, about 50 μl, about 75 μl, about 100 μl, about 300 μl, about 1 ml, about 3 ml, about 10 ml, about 30 ml, about 50 ml or about 100 ml. In certain embodiments, the reaction volume of an RPA process of the present disclosure can be from about 50 μl to about 100 μl.

In certain embodiments, the target nucleic acid can be of any concentration in the RPA reaction. For example, but not by way of limitation, there can be less than about 10,000 copies of the target nucleic acid, less than about 1000 copies of the target nucleic acid, less than about 100 copies of the target nucleic acid, less than about 10 copies of the target nucleic acid or 1 copy of the target nucleic acid in an RPA reaction. In certain embodiments, an isothermal amplification process of the present disclosure can result in a 10 fold, 100 fold, 1000 fold, 10,000 fold, 100,000 fold or 1,000,000 fold amplification of the target nucleic acid in the reaction.

In certain embodiments, the reaction temperature of an RPA process of the present disclosure is between about 20 °C to about 50 °C, about 20 °C to about 40 °C, about 20 °C to about 30 °C or about 37 °C to about 42 °C. In certain embodiments, the reaction temperature is about 40 °C.

In certain embodiments, the reaction time of an RPA process of the present disclosure is about 10 minutes to about 3 hours, about 10 minutes to about 2 hours, about 10 minutes to about 1 hour, is about 10 minutes to about 30 minutes, or in about 10 minutes to about 25 minutes, or about 10 minutes to about 20 minutes, or even about 10 minutes to about 15 minutes from the addition of the reagents sufficient to initiate the RPA process. In certain embodiments, the reaction time of an RPA process of the present disclosure is about 1 minute to about 20 minutes, about 5 minutes to about 20 minutes, about 8 minutes to about 20 minutes, about 1 minute to about 10 minutes or about 5 minutes to about 10 minutes from the addition of the reagents sufficient to initiate RPA amplification. In certain embodiments, the reaction time of an RPA process of the present disclosure is about 1 minute. In certain embodiments, the reaction time of an RPA process of the present disclosure is about 5 minutes. In certain embodiments, the reaction time of an RPA process of the present disclosure is about 20 minutes. In certain embodiments, the RPA reaction time is sufficient to obtain a result, e.g., detection of a target nucleic acid.

### B. NEAR Process

In certain embodiments, the isothermal amplification process is (NEAR). In certain embodiments, the present disclosure provides a NEAR process and/or reagent composition for a NEAR process that includes trehalose and PEG at the ratios and amounts described herein. In certain embodiments, the present disclosure provides a NEAR process that is performed in the presence of and/or uses a reagent composition that includes a trehalose to PEG wt % ratio of about 0.3 to about 0.79. For example, but not by way of limitation, the present disclosure provides a NEAR process that is performed in the presence of and/or uses a reagent composition that includes a trehalose to PEG wt % ratio of about 0.45 to about 0.75, e.g., about 0.5 to about 0.7.

In certain embodiments, the present disclosure provides a NEAR process that is performed in the presence of and/or uses a reagent composition that includes a trehalose to PEG wt % ratio of about 0.79 or less. In certain embodiments, the present disclosure provides a NEAR process that is performed in the presence of and/or uses a reagent composition that includes a trehalose to PEG wt % ratio of about 0.75 or less, e.g., 0.65 or less or 0.55 or less.

In certain embodiments, the present disclosure provides a NEAR process that is performed in the presence of about 2% to about 9% w/v of PEG. In certain embodiments, the present disclosure provides a NEAR process that is performed in the presence of about 2% to about 8% w/v of PEG, e.g., about 5% to about 8% w/v of PEG.

In certain embodiments, the present disclosure provides a NEAR process that is performed in the presence of about 1% to about 12% w/v of trehalose, e.g., about 2% to about 12% w/v of trehalose. In certain embodiments, the present disclosure provides a NEAR process that is performed in the presence of about 5% to about 12% w/v of trehalose. In certain embodiments, the present disclosure provides a NEAR process that is performed in the presence of about 3% to about 5% w/v of trehalose, e.g., about 4% w/v of trehalose.

In NEAR, a target nucleic acid sequence, having a sense and antisense strand, is contacted with a pair of amplification oligonucleotides. The first amplification oligonucleotide comprises a nucleic acid sequence comprising a recognition region at the 3′ end that is complementary to the 3′ end of the target sequence antisense strand, a nicking enzyme site upstream of said recognition region, and a stabilizing region upstream of said nicking enzyme site (see, e.g., U.S. Pat. Nos. 9,689,031; 9,617,586; 9,562,264; and 9,562,263, each of which is incorporated herein by reference in its entirety). The second amplification oligonucleotide comprises a nucleotide sequence comprising a recognition region at the 3′ end that is complementary to the 3′ end of the target sequence sense strand, a nicking enzyme site upstream of said recognition region, and a stabilizing region upstream of said nicking enzyme site. Two nicking enzymes are provided. One nicking enzyme is capable of nicking at the nicking enzyme site of the first amplification oligonucleotide but incapable of nicking within said target sequence. The other nicking enzyme is capable of nicking at the nicking enzyme site of the second amplification oligonucleotide but incapable of nicking within said target sequence. A DNA polymerase is employed under conditions for amplification which involves multiple cycles of extension of the amplification oligonucleotides thereby producing a double-stranded nicking enzyme site which are nicked by the nicking enzymes to produce the amplification product. For example, see U.S. Pat. Nos. 9,689,031; 9,617,586; 9,562,264; 9,562,263; and 10,851,406 and U.S. patent application Ser. Nos. 15/467,893 and 16/243,829, each of which is incorporated herein by reference in its entirety.

In certain embodiments, reactions use only two templates to prime, one or two nicking enzymes and a polymerase, under isothermal conditions. In exemplary non-limiting embodiments, the polymerase and the nicking enzyme are thermophilic, and the reaction temperature is significantly above the melting temperature of the hybridized target region. The nicking enzyme nicks only one strand in a double-stranded duplex, so that incorporation of modified nucleotides is not necessary as it is in strand displacement. In certain embodiments, the method is able to amplify RNA without a separate reverse transcription step, although conversion of RNA to DNA by reverse transcription may be used if desired.

In certain embodiments, the method comprises contacting a target DNA molecule comprising a double-stranded target sequence having a sense strand and an antisense strand, with a forward template and a reverse template, wherein said forward template comprises a nucleic acid sequence comprising a recognition region at the 3′ end that is complementary to the 3′ end of the target sequence antisense strand; a nicking enzyme site upstream of said recognition region, and a stabilizing region upstream of said nicking enzyme site; the reverse template comprises a nucleotide sequence comprising a recognition region at the 3′ end that is complementary to the 3′ end of the target sequence sense strand, a nicking enzyme site upstream of the recognition region, and a stabilizing region upstream of the nicking enzyme site; providing a first nicking enzyme that is capable of nicking at the nicking enzyme site of the forward template, and does not nick within the target sequence; providing a second nicking enzyme that is capable of nicking at the nicking enzyme site of the reverse template and does not nick within the target sequence; and providing a DNA polymerase; under conditions wherein amplification is performed by multiple cycles of the polymerase extending the forward and reverse templates along the target sequence producing a double-stranded nicking enzyme site, and the nicking enzymes nicking at the nicking enzyme sites, producing an amplification product.

In certain embodiments, the DNA polymerase is a thermophilic polymerase. In other examples, the polymerase and said nicking enzymes are stable at temperatures up to 37 °C, 42 °C, 60 °C, 65 °C, 70 °C, 75 °C, 80 °C or 85 °C. In certain embodiments, the polymerase is stable up to 60 °C. In certain embodiments, the polymerase can, for example, be selected from the group consisting of Bst (large fragment), 9° N, VentR® (exo-) DNA Polymerase, THERMINATOR, and THERMINATOR II (New England Biolabs).

In certain embodiments, the nicking enzyme can, for example, nick upstream of the nicking enzyme binding site, or the nicking enzyme may nick downstream of the nicking enzyme binding site. In certain embodiments, the forward and reverse templates comprise nicking enzyme sites recognized by the same nicking enzyme and the first and the second nicking enzyme are the same. In certain embodiments, the nicking enzyme can, for example, be selected from the group consisting of Nt. BspQI, Nb.BbvCi, Nb.BsmI, Nb.BsrDI, Nb.BtsI, Nt.AlwI, Nt.BbvCI, Nt.BstNBI, Nt. CviPII, Nb.Bpu10I and Nt. Bpu10I.

In certain embodiments, the target sequence includes from 1 to 5 nucleotides more than the sum of the nucleotides of said forward template recognition region and said reverse template recognition region.

In certain embodiments, the forward template is provided at the same concentration as the reverse template. In certain embodiments, the forward template is provided at a ratio to the reverse template at the range of ratios of 1:100 to 100:1.

In certain embodiments, the NEAR reaction time can be about 10 minutes to about 3 hours, about 10 minutes to about 2 hours, about 10 minutes to about 1 hour, about 10 minutes to about 30 minutes, or about 8 minutes to about 25 minutes, or about 8 minutes to about 20 minutes, or even about 8 minutes to about 15 minutes from the addition of the reagents sufficient to initiate NEAR amplification. In certain embodiments, the NEAR reaction time is about 1 minute to about 20 minutes, about 5 minutes to about 20 minutes, about 8 minutes to about 20 minutes, about 1 minute to about 10 minutes or about 5 minutes to about 10 minutes from the addition of the reagents sufficient to initiate NEAR amplification.

### C. Nucleic Acid Detection

Nucleic acid detection as employed herein is used to determine presence (e.g., presence or absence) of a target nucleic acid or a plurality of different target nucleic acids in a sample. In certain embodiments, nucleic acid detection is employed to quantify the amount of a nucleic acid or a plurality of different nucleic acids in a sample. As embodied herein, the nucleic acid detection methods of the present disclosure can be configured to detect a target nucleic acid or plurality of different target nucleic acids using any of a variety or combination of suitable detection techniques.

In certain embodiments, following amplification of one or a plurality of target nucleic acids present in the sample, the methods of the present disclosure can be configured to detect the amplified nucleic acid(s), e.g., via hybridization. For example, but not limitation, such detection can comprise hybridizing a probe oligonucleotide sufficiently complementary to an amplified target nucleic acid to facilitate detection of the target nucleic acid. In certain embodiments, following hybridization of the probe oligonucleotide to the target nucleic acid, the method comprises detecting hybridization of the probe oligonucleotide to the target nucleic acid. For example, but not limitation, such detection can be achieved by observing a signal from a detectable label, whereby (i) the presence of one or more signals indicates hybridization of the probe oligonucleotide to the target nucleic acid and is indicative of the presence of the target nucleic acid in the sample, and (ii) the absence of a signal indicates the absence of the target nucleic acid in the sample. Detection of a signal from the probe oligonucleotide can be performed using a variety of suitable methodologies, depending on the type of detectable label.

In certain embodiments, nucleic acid amplification, e.g., using an isothermal amplification process as described herein, and nucleic acid detection can occur simultaneously, e.g., during an amplification and detection process. In certain embodiments, an amplification and detection process as disclosed herein includes the simultaneous amplification and detection of nucleic acids in a sample, e.g., an eluate. In certain embodiments, the amplification and detection process begins with the incubation of an eluate with the reagents sufficient to initiate amplification of a target nucleic acid in the sample using the isothermal amplification methods of the present disclosure, if present, and ends with the determination of a result in the sample, e.g., the detection of the target nucleic acid in the eluate or the lack of detection of the target nucleic acid in the sample.

In certain embodiments, detection of amplified nucleic acids can employ optical detection, digital detection and/or other detection methods known in the art.

i. Optical Detection

In certain embodiments, detection of amplified nucleic acids can be performed using optical detection. For example, but not by way of limitation, the detection of the amplified target nucleic acid is mediated by the binding of a labeled probe or by incorporation of a label into amplified copies of the target nucleic acid.

Additionally, or alternatively, and in accordance with another aspect of the disclosed subject matter, detection is mediated by observation of a fluorescent label (such as fluorescein (e.g., 5-fluorescein, 6-carboxyfluorescein (e.g., FAM), 3′6-carboxyfluorescein, 5 (6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), Fluor Orange 560 fluorophore, Quasar 670 fluorophore and Quasar 705 fluorophore. An introduction to labels, labeling procedures and detection of labels is found in Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc., Eugene, Oreg. A fluorescent label can be used in FPIA (see, e.g., U.S. Pat. Nos. 5,593,896, 5,573,904, 5,496,925, 5,359,093, and 5,352,803, which are hereby incorporated by reference in their entireties).

Additionally, or alternatively, and in accordance with another aspect of the disclosed subject matter, optical detection is performed using fluorescence, chemiluminescence, or other means of generating a signal in response to the presence of an analyte. Many assays are performed by measuring the intensity of a light signal generated in the total volume of a reaction mixture. The light signal generated can be measured by an optical means, wherein the light signal generated is emitted by a large number of molecules. Typically, as described herein, assays can involve combining a sample suspected of containing a target nucleic acid, e.g., target nucleic acids amplified as described herein, with a reagent comprising a labeled probe capable of hybridizing with the target nucleic acid to form a reaction mixture. The signal attributable to the label is then measured after unbound probe is removed from the reaction mixture, typically by performing a wash step. In certain embodiments, the presence of a detectable signal is sufficient to confirm the presence of the target nucleic acid in the sample. In certain embodiments, the signal that is derived from the total volume of the reaction mixture is measured and then compared to a calibration curve to establish the concentration of target nucleic acid present in the sample.

Additionally, or alternatively, and in accordance with another aspect of the disclosed subject matter, the optical detection strategy comprises the use of probes labeled with both a detectable label and a “quencher molecule” where the quencher molecule is capable of interacting with a detectable label to reduce or eliminate the signal emitted by the detectable label. For example, but not by way of limitation, a detection probe employed in the methods of the present disclosure can have a fluorescent moiety that is covalently linked, e.g., to the 5′ end of the probe, and has a quencher molecule, e.g., at the 3′ end of the probe. In the absence of target sequences, the probe adopts a conformation that brings the quencher close enough to the excited fluorophore to absorb its energy before it can be fluorescently emitted. When the probe binds to its complementary sequence in the target, the fluorophore and the quencher are positioned at a sufficient distance apart to allow fluorescent emission and detection. In certain embodiments, the quencher can be selected from any suitable quencher known in the art, such as, for example, BLACK HOLE QUENCHER® 1 (BHQ-1®), BLACK HOLE QUENCHER® 2 (BHQ-28), BLACK HOLE QUENCHER®-1-dT (BHQ-1 dTR), BLACK HOLE QUENCHER®-2-dT (BHQ-2dT®), IOWA BLACK® FQ, and IO WA BLACK® RQ. For example, but not by way of limitation, an oligonucleotide probe used in the methods of the present disclosure can comprise a FAM fluorophore and a BHQ-1 dT® quencher or a BHQ-2dT® quencher. In certain embodiments, an oligonucleotide probe used in the methods of the present disclosure can include a Quasar 670 fluorophore and a BHQ-1® quencher or a BHQ-2® quencher. In certain embodiments, an oligonucleotide probe used in the methods of the present disclosure can include a Quasar 670 fluorophore and a BHQ-1 dT® quencher or a BHQ-2dT® quencher.

Additionally, or alternatively, and in accordance with another aspect of the disclosed subject matter, specific probes, e.g., probes for specific target nucleic acids and/or internal controls, are each labeled with a different fluorophore, thus allowing for simultaneous detection of a plurality of amplified products.

Additionally, or alternatively, and in accordance with another aspect of the disclosed subject matter, light intensity can be measured using light emitting diodes (LEDs) and/or lasers for excitation and any suitable detector for detection emissions. Fluorescence-optical detection “scanners” can be used which scan the surface of the chip using a focused laser beam, allowing for detection of the emitted fluorescence light. Exemplary fluorescence scanners are described in, e.g., U.S. Pat. Nos. 5,837,475 and 5,945,679. Scanners in which a confocal excitation and detection system has been integrated into an epifluorescence microscope are also known. The systems used in scanners for detecting the emitted fluorescence light are usually “one-channel systems”, i.e., for example, individual photocells or secondary electron multipliers (photomultipliers). Two-dimensional detection systems such as, for example, charged-coupled device (CCD) cameras, also are used for detecting fluorescence or chemiluminescent light of a sample. Commercially available systems have either an optical imaging system which projects the binding surface provided with chemiluminescent markers or fluorescent markers on a CCD sensor by using lens optics, or a combination of image intensifier and CCD camera.

ii. Digital Detection

In certain embodiments, detection of amplified nucleic acids can be performed using digital detection methods. Because every single target nucleic acid, as an end-point entity, can be detected in principle in the context of digital detection, the components and methods associated with digital detection can significantly increase detection sensitivity for sample analysis compared to systems using analog optical detection. As such, digital detection can be performed using a lower concentration of analyte, e.g., target nucleic acids, which can allow for decreased time to process the sample for detection. Additionally, or alternatively, detection can be performed using a smaller sample volume, less reagent material, less conjugate material, fewer microparticles, or any combination of these, which can reduce costs to perform each assay. As such, and as described herein, sample preparation time can be improved due at least in part to less sample manipulation involved (e.g., faster washing times) and/or improved kinetics of reactions achieved using a lower sample volume, less reagent or conjugate material, and/or fewer particles or beads to obtain an analyte concentration suitable for detection. Assays using less sample volume and/or reagent material can be performed using smaller equipment, which can reduce the footprint of the laboratory system for performing the assays as discussed further herein. In addition, or as a further alternative, increased detection sensitivity can provide additional benefits when used with multiplexing. For example, and without limitation, when multiple analytes and corresponding signals are combined into a single, multiplexed assay, a noise level associated with the detection of each analyte signal can be multiplied to obtain a total noise level of the multiplexed system. By increasing the detection sensitivity of each signal being detected, the improved sensitivity can be multiplied to further reduce the total noise level of the multiplexed system.

Digital detection can provide increased sensitivity due at least in part to a reduction of noise during detection relative to the signal being measured, for example, producing a higher signal-to-noise ratio. Such improved signal-to-noise ratios are possible by coupling the analyte of interest, e.g., a particular target nucleic acid, to an independently detectable end-point entity. For example, but not limitation, amplified target nucleic acids can be immobilized to microparticles and labeled with detectable conjugates, where the conjugate is a detectable end-point entity in that it can emit an independently detectable signal, either directly or via the conversion of a substrate.

In certain embodiments, the detection operation employs a digital nanowell detection process. In certain embodiments, a support medium, such as, but not limited to, microparticles, beads, or other labels, can be mixed with the sample in order to perform the digital detection process after amplification. In certain embodiments, reagents including antibodies and coated microparticles can be combined.

For example, but not by way of limitation, digital nanowell detection processes incorporating microparticles can employ anti-Digoxin microparticles. In certain embodiments, digital nanowell detection incorporating microparticles can be performed in a formulation comprising: Tris-HCl, NaCl, BSA, Tergitol 15-s-40, sodium azide and 0.02% anti-Digoxin μP (microparticles). For example, but not by way of limitation, digital nanowell detection incorporating microparticles can be performed in the following context: about 50 mM Tris-HCl at a pH of about 8.0; about 150 mM NaCl; about 0.2% BSA; about 0.5% Tergitol 15-s-40; about 0.08% Sodium azide; and about 0.02% anti-Digoxin μP (microparticles). The solution can be washed, for example to remove excess reagents and/or unbound analyte. Any suitable number of washes can be performed for each washing step, including one, two, or three or more washes, and each wash can be performed in a single chamber or location or among different chambers or locations. For example, and not limitation, as embodied herein, three washes can be performed.

Additionally, or alternatively, and in accordance with another aspect of the disclosed subject matter, a conjugate can be added to bind with an analyte of interest in the sample. In certain embodiments, a conjugate, e.g., Alkaline Phosphatase-SA, can be added to the sample. In certain embodiments, additional reagents including, but not limited to, Tris-HCl, NaCl, MgCl₂, ZnCl₂, fish gelatin, Rabbit IgG, Saponin, calf serum, Goat IgG and Sodium azide, can be added to the sample. For example, and not limitation, the conjugate can include one or more reagents or enzymes selected or configured to react with the analyte of interest to produce a signal for detection by the detection component. In certain embodiments, the digital nanowell detection process will employ conjugates in the following context: about 3000 μM Alkaline Phosphatase-SA; about 100 mM Tris-HCl, at a pH of about 7.5; about 500 mM NaCl; about 1 mM MgCl₂; about 0.1 ZnCl₂; about 8.9 g/L fish gelatin; about 30 μg/mL of Rabbit IgG; about 0.1% Saponin; about 10% calf serum; about 5 mg/mL Goat IgG; and about 0.1% Sodium azide. The solution can be washed, for example to remove excess conjugate unbound to the analyte of interest. Any suitable number of washes can be performed for each washing step, including one, two, or three or more washes, and each wash can be performed in a single chamber or location or among different chambers or locations.

Additionally, or alternatively, and in accordance with another aspect of the disclosed subject matter, detection of the microparticles bound with analytes and conjugates can be performed in a single chamber or location or among different chambers or locations. For purpose of illustration and not limitation, the detection chamber or location can include a surface and a detection region. The microparticles can be added to the detection chamber or location using any suitable technique, including but not limited to pipetting, magnetic force or dielectrophoresis. In certain embodiments, the digital nanowell detection process will employ a detection substrate, e.g., AJ Phos. In certain embodiments, additional reagents including, but not limited to, DEA, MgCl₂ and Tween 20, can be added in combination with the detection substrate. For example, but not by way of limitation, the digital nanowell detection process will employ a detection substrate in the following context: about 200 μM AJ Phos; about 1 M DEA; about 1 mM MgCl₂; and about 0.05% Tween 20. As embodied herein, the detection region can include one or more nanowells. The microparticles can be moved to the detection region, for example and as embodied herein, an array of nanowells. The microparticles can be moved to the nanowells using any suitable technique, including but not limited to pipetting, magnetic force or dielectrophoresis. In certain embodiments, oil, e.g., 3 mM Guaiazulene in FC-40 oil, is added to seal the nanowells. In certain embodiments, a dye can be added to increase contrast or otherwise improve optical conditions for detection of the analyte of interest in the nanowells. In certain embodiments, the digital nanowell detection process incorporating microparticles will employ a dye in the following context: about 0.1% Tween 20; about 10 mM PBS; and about 50 mM Nigrosine. In certain embodiments, one or more images of the microparticles are taken and analyzed to determine the presence or absence of the analyte of interest and/or a concentration of the analyte of interest in the sample.

### D. Samples

In certain embodiments, the target nucleic acids that can be amplified using the disclosed methods can be isolated from a sample, e.g., a sample from a subject. Target nucleic acids can be isolated from a sample by any method known in the art. Non-limiting examples of methods for isolating nucleic acids from a sample are disclosed in International Patent Application PCT/US2022/027067, which is incorporated herein by reference in its entirety. For example, but not by way of limitation, nucleic acids can be isolated from a sample by the use of magnetic microparticles (e.g., copper titanium microparticles), e.g., as shown in FIGS. 4, 9 and 10 of PCT/US2022/027067.

In certain embodiments, the sample can be a tissue sample. In certain embodiments, the sample can be obtained from preserved tissue, e.g., fixed tissue, from frozen tissue or from fresh tissue, e.g., fresh tissue samples. Non-limiting examples of tissues include eye, muscle, skin, tendon, vein, artery, heart, spleen, lymph node, bone, bone marrow, lung, bronchi, trachea, gut, small intestine, large intestine, colon, rectum, salivary gland, tongue, gallbladder, appendix, liver, pancreas, brain, stomach, skin, kidney, ureter, bladder, urethra, gonad, testicle, ovary, uterus, fallopian tube, thymus, pituitary, thyroid, adrenal or parathyroid tissue. In certain embodiments, the tissue can be cancerous tissue, e.g., tumor tissue.

In certain embodiments, the sample is a biological fluid sample. In certain embodiments, the biological fluid sample is a bodily secretion. Non-limiting examples of biological fluid and bodily secretion samples include blood (e.g., whole blood, lysed whole blood, serum or plasma), saliva or oral fluid, sweat, tears, mucus, urine, lymphatic fluid, cerebrospinal fluid, interstitial fluid, bronchoalveolar lavage fluid, nasopharyngeal fluid or any other sample suitable for analysis using the methods and techniques described herein. In certain embodiments, the sample can be a nasal swab (e.g., a nasal swab (e.g., at least partially coated with a target nucleic acid) in a buffer) and/or a sample obtained using a nasal swab (e.g., a nasopharyngeal fluid sample).

In certain embodiments, the biological fluid sample is whole blood. As used herein, “whole blood” refers to blood that has not had any components removed (blood that contains both the fluid and solid components). Transfusion of whole blood, or the red blood cell (RBC) component of whole blood, can increase a patient's oxygen-carrying capacity by effectively increasing the patient's RBC count to thereby increase the amount of available oxygen-carrying hemoglobin. In addition to its oxygen-carrying capacity, whole blood transfusions can be a source of platelets, which aid in blood clotting. In certain embodiments, the clinical use, transfusion of platelets can be used to treat thrombocytopenia, certain cancers, aplastic anemia as well as marrow transplants.

In certain embodiments, biological fluid sample is lysed whole blood. As used herein, “lysed whole blood” refers to blood that has not had any components removed (blood that contains both the fluid and solid components), but where the RBCs have been lysed by exposure to, e.g., a buffer comprising ammonium chloride, potassium carbonate and EDTA. Ammonium chloride, which lyses RBCs, has minimal effect on lymphocytes.

In certain embodiments, the biological fluid sample is plasma. Plasma is the aqueous portion of blood that remains after centrifugation to remove the cellular components of blood. Plasma can, in certain embodiments, include albumin, coagulation factors, fibrinolytic proteins, immunoglobulin and other proteins. Products derived from plasma donation can, in certain embodiments, be used to treat bleeding disorders and/or life-threatening trauma/hemorrhages.

In certain embodiments, the biological fluid sample is serum. As used herein, “serum” is the clear portion of plasma that does not contain fibrinogen, cells or any solid elements.

In certain embodiments, the sample is obtained from a subject. In certain embodiments, the subject is a vertebrate or an invertebrate, such as a human or non-human animal, for example, a mammal. In certain embodiments, non-human animal subjects include rodents such as mice, rats, hamsters, guinea pigs, rabbits, dogs, cats, sheep, pigs, goats, cattle, horses, apes and monkeys. In certain embodiments, the subject is a human.

### III. COMPOSITIONS

The present disclosure further provides compositions for performing the methods of the present disclosure. For example, but not by way of limitation, the present disclosure provides compositions comprising one or more reagents, e.g., reagent compositions, for performing an isothermal application process of the present disclosure.

In certain embodiments, a composition of the present disclosure includes trehalose and PEG at the ratios and amounts described herein, e.g., the ratios and amounts described in Section II. In certain embodiments, a composition of the present disclosure includes trehalose and PEG at a wt % ratio of about 0.79 or less or about 0.75 or less, e.g., about 0.74 or less, about 0.73 or less, about 0.72 or less, about 0.71 or less, about 0.7 or less, about 0.69 or less, about 0.68 or less, about 0.67 or less, about 0.66 or less, about 0.65 or less, about 0.64 or less, about 0.63 or less, about 0.62 or less, about 0.61 or less, about 0.60 or less, about 0.59 or less, about 0.58 or less, about 0.57 or less, about 0.56 or less, about 0.55 or less, about 0.54 or less, about 0.53 or less, about 0.52 or less, about 0.51 or less, about 0.50 or less, about 0.49 or less, about 0.48 or less, about 0.47 or less, about 0.46 or less, about 0.45 or less, about 0.44 or less, about 0.43 or less, about 0.42 or less, about 0.41 or less, about 0.40 or less, about 0.39 or less, about 0.38 or less, about 0.37 or less, about 0.36 or less, about 0.35 or less, about 0.34 or less, about 0.33 or less, about 0.32 or less or about 0.31 or less. In certain embodiments, a composition of the present disclosure includes trehalose and PEG at a wt % ratio of about 0.65 or less or 0.55 or less.

In certain embodiments, a composition of the present disclosure includes trehalose and PEG at a wt % ratio of about 0.75 or less.

In certain embodiments, a composition of the present disclosure includes trehalose and PEG at a wt % ratio of about 0.65 or less.

In certain embodiments, a composition of the present disclosure includes trehalose and PEG at a wt % ratio of about 0.60 or less.

In certain embodiments, a composition of the present disclosure includes trehalose and PEG at a wt % ratio of about 0.55 or less.

In certain embodiments, a composition of the present disclosure includes trehalose and PEG at a wt % ratio of about 0.45 or less.

In certain embodiments, a composition of the present disclosure includes trehalose and PEG at a wt % ratio of about 0.35 or less.

In certain embodiments, a composition of the present disclosure includes a trehalose to PEG wt % ratio of no greater than about 0.79 or no greater than about 0.75, e.g., no greater than about 0.74, no greater than about 0.73, no greater than about 0.72, no greater than about 0.71, no greater than about 0.7, no greater than about 0.69, no greater than about 0.68, no greater than about 0.67, no greater than about 0.66, no greater than about 0.65, no greater than about 0.64, no greater than about 0.63, no greater than about 0.62, no greater than about 0.60, no greater than about 0.59, no greater than about 0.58, no greater than about 0.57, no greater than about 0.56, no greater than about 0.55, no greater than about 0.54, no greater than about 0.53, no greater than about 0.52, no greater than about 0.51, no greater than about 0.50, no greater than about 0.49, no greater than about 0.48, no greater than about 0.47, no greater than about 0.46, no greater than about 0.45, no greater than about 0.44, no greater than about 0.43, no greater than about 0.42, no greater than about 0.41, no greater than about 0.40, no greater than about 0.39, no greater than about 0.38, no greater than about 0.37, no greater than about 0.36, no greater than about 0.35, no greater than about 0.34, no greater than about 0.33, no greater than about 0.32 or no greater than about 0.31.

In certain embodiments, a composition of the present disclosure includes a trehalose to PEG wt % ratio of no greater than about 0.75.

In certain embodiments, a composition of the present disclosure includes a trehalose to PEG wt % ratio of no greater than about 0.65.

In certain embodiments, a composition of the present disclosure includes a trehalose to PEG wt % ratio of no greater than about 0.55.

In certain embodiments, a composition of the present disclosure includes a trehalose to PEG wt % ratio no greater than about 0.45.

In certain embodiments, a composition of the present disclosure includes a trehalose to PEG wt % ratio no greater than about 0.35. In certain embodiments, a composition of the present disclosure includes trehalose and PEG at a wt % ratio of about 0.3 to about 0.79, e.g., about 0.3 to about 0.7, about 0.3 to about 0.5 or about 0.3 to 0.35. In certain embodiments, a composition of the present disclosure includes trehalose and PEG at a wt % ratio of about 0.45 to about 0.75, e.g., e.g., about 0.45 to about 0.75, about 0.5 to about 0.75, about 0.55 to about 0.75, about 0.6 to about 0.75, about 0.65 to about 0.75, about 0.7 to about 0.75, about 0.4 to about 0.7, about 0.4 to about 0.65, about 0.4 to about 0.6, about 0.4 to about 0.55, about 0.4 to about 0.45, about 0.5 to about 0.7, about 0.45 to about 0.55, about 0.45 to about 0.65, about 0.55 to about 0.65, about 0.5 to about 0.55, about 0.6 to about 0.65 or about 0.7 to about 0.75. In certain embodiments, a composition of the present disclosure includes trehalose and PEG at a wt % ratio of about 0.3 to about 0.7. In certain embodiments, a composition of the present disclosure includes trehalose and PEG at a wt % ratio of about 0.5 to about 0.75. In certain embodiments, a composition of the present disclosure includes trehalose and PEG at a wt % ratio of about 0.5 to about 0.7.

In certain embodiments, a composition of the present disclosure includes trehalose and PEG at a wt % ratio of about 0.3 to about 0.75.

In certain embodiments, about 5%, about 10%, about 15%, about 20%, about 25%, about 30%, about 35%, about 40%, about 45%, about 50%, about 55%, about 60%, about 65%, about 70% or about 75% of a composition of the present disclosure is added to an isothermal amplification process, e.g., added to a solution to prepare a master mix for performing the isothermal amplification process. In certain embodiments, the present disclosure provides a composition that when added to an isothermal amplification process (e.g., diluted) results in a concentration of about 2% to about 9% w/v of PEG, e.g., about 5% to about 9% w/v of PEG or about 6% to about 9% w/v of PEG. In certain embodiments, the present disclosure provides a composition that when added to an isothermal amplification process (e.g., diluted) results in a concentration of about 2% to about 8.5% w/v of PEG, e.g., about 5% to about 8.5% w/v of PEG or about 6% to about 8.5% w/v of PEG. In certain embodiments, the present disclosure provides a composition that when added to an isothermal amplification process (e.g., diluted) results in a concentration of about 2% to about 8.5% w/v of PEG, e.g., about 5% to about 8.5% w/v of PEG or about 6% to about 8.5% w/v of PEG. In certain embodiments, the present disclosure provides a composition that when added to an isothermal amplification process (e.g., diluted) results in a concentration of about 5% to about 8.5% w/v of PEG. In certain embodiments, the present disclosure provides a composition that when added to an isothermal amplification process (e.g., diluted) results in a concentration of about 6.3% w/v of PEG. In certain embodiments, the present disclosure provides a composition that when added to an isothermal amplification process (e.g., diluted) results in a concentration of about 6.5% w/v of PEG. In certain embodiments, the present disclosure provides a composition that when added to an isothermal amplification process (e.g., diluted) results in a concentration of about 7% of PEG. In certain embodiments, the present disclosure provides a composition that when added to an isothermal amplification process (e.g., diluted) results in a concentration of about 7.7% of PEG. In certain embodiments, the present disclosure provides a composition that when added to an isothermal amplification process (e.g., diluted) results in a concentration of about 8.4% of PEG. For example, but not by way of limitation, about 5%, about 10%, about 15%, about 20%, about 25%, about 30%, about 35%, about 40%, about 45%, about 50%, about 55%, about 60%, about 65%, about 70% or about 75% of a composition of the present disclosure is added to an isothermal amplification process to result in a reaction concentration of about 5% to about 8.5% w/v of PEG. In certain embodiments, about 5%, about 10%, about 15%, about 20%, about 25%, about 30%, about 35%, about 40%, about 45%, about 50%, about 55%, about 60%, about 65%, about 70% or about 75% of a composition of the present disclosure is added to an isothermal amplification process to result in a reaction concentration of about 5% to about 9% w/v of PEG. In certain embodiments, about 5% or less, about 10% or less, about 15% or less, about 20% or less, about 25% or less, about 30% or less, about 35% or less, about 40% or less, about 45% or less, about 50% or less, about 55% or less, about 60% or less, about 65% or less, about 70% or less or about 75% or less of a composition of the present disclosure is added to an isothermal amplification process to result in a reaction concentration of about 5% to about 9% w/v of PEG. In certain embodiments, about 5% or less, about 10% or less, about 15% or less, about 20% or less, about 25% or less, about 30% or less, about 35% or less, about 40% or less, about 45% or less, about 50% or less, about 55% or less, about 60% or less, about 65% or less, about 70% or less or about 75% or less of a composition of the present disclosure is added to an isothermal amplification process to result in a reaction concentration of about 5% to about 8.5% w/v of PEG.

In certain embodiments, a reagent composition, e.g., a reagent composition including one or more non-protein components, can include a concentration of about 10% to about 30% w/v of PEG to result in a reaction concentration of about 5% to about 8.5% w/v of PEG. In certain embodiments, a reagent composition, e.g., a reagent composition including one or more non-protein components, can include a concentration of about 10% to about 25% w/v of PEG to result in a reaction concentration of about 5% to about 8.5% w/v of PEG. In certain embodiments, a reagent composition, e.g., a reagent composition including one or more non-protein components, can include a concentration of about 10% to about 20% w/v of PEG to result in a reaction concentration of about 5% to about 8.5% w/v of PEG.

In certain embodiments, the present disclosure provides a composition comprising about 10% to about 30% w/v of PEG.

In certain embodiments, the present disclosure provides a composition comprising about 10% to about 25% w/v of PEG, e.g., about 30% w/v of PEG.

In certain embodiments, the present disclosure provides a composition comprising about 10% to about 20% w/v of PEG.

In certain embodiments, the present disclosure provides a composition comprising about 2% to about 9% w/v of PEG. In certain embodiments, the present disclosure provides a composition comprising about 2% to about 8.5% w/v of PEG. In certain embodiments, the present disclosure provides a composition comprising about 4% to about 6% w/v of PEG. In certain embodiments, the present disclosure provides a composition comprising about 5% to about 7% w/v of PEG. In certain embodiments, the present disclosure provides a composition comprising about 6% to about 7% w/v of PEG. In certain embodiments, the present disclosure provides a composition comprising about 6% to about 8% w/v of PEG. In certain embodiments, the present disclosure provides a composition comprising about 7% to about 8% w/v of PEG. In certain embodiments, the present disclosure provides a composition comprising about 5.5% w/v of PEG. In certain embodiments, the present disclosure provides a composition comprising about 6.5% w/v of PEG. In certain embodiments, the present disclosure provides a composition comprising about 7.7% w/v of PEG. In certain embodiments, the present disclosure provides a composition comprising about 5% to about 8% w/v of PEG. In certain embodiments, the present disclosure provides a composition comprising about 8% to about 9% w/v of PEG. In certain embodiments, the present disclosure provides a composition comprising about 8% to about 8.5% w/v of PEG.

In certain embodiments, about 5%, about 10%, about 15%, about 20%, about 25%, about 30%, about 35%, about 40%, about 45%, about 50%, about 55%, about 60%, about 65%, about 70% or about 75% of a composition of the present disclosure is added to an isothermal amplification process, e.g., to reduce the concentration of trehalose provided by the composition. In certain embodiments, the present disclosure provides a composition that when added to an isothermal amplification process (e.g., diluted) results in a concentration of about 1.5% to about 6% w/v of trehalose. In certain embodiments, the present disclosure provides a composition that when added to an isothermal amplification process (e.g., diluted) results in a concentration of about 2% to about 6% w/v of trehalose. For example, but not by way of limitation, about 5%, about 10%, about 15%, about 20%, about 25%, about 30%, about 35%, about 40%, about 45%, about 50%, about 55%, about 60%, about 65%, about 70% or about 75% of a composition of the present disclosure is added to an isothermal amplification process, e.g., to reduce the concentration of trehalose provided by the composition to a concentration of about 2% to about 6% w/v of trehalose, e.g., about 2% to about 4% trehalose. In certain embodiments, about 5%, about 10%, about 15%, about 20%, about 25%, about 30%, about 35%, about 40%, about 45%, about 50%, about 55%, about 60%, about 65%, about 70% or about 75% of a composition of the present disclosure is added to an isothermal amplification process, e.g., to reduce the concentration of trehalose provided by the composition to a concentration of about 1.5% to about 6% w/v of trehalose, e.g., about 1.5% to about 4% trehalose. In certain embodiments, about 5% or less, about 10% or less, about 15% or less, about 20% or less, about 25% or less, about 30% or less, about 35% or less, about 40% or less, about 45% or less, about 50% or less, about 55% or less, about 60% or less, about 65% or less, about 70% or less or about 75% or less of a composition of the present disclosure is added to an isothermal amplification process, e.g., to reduce the concentration of trehalose provided by the composition to a concentration of about 2% to about 6% w/v of trehalose, e.g., about 2% to about 4% trehalose. In certain embodiments, about 5% or less, about 10% or less, about 15% or less, about 20% or less, about 25% or less, about 30% or less, about 35% or less, about 40% or less, about 45% or less, about 50% or less, about 55% or less, about 60% or less, about 65% or less, about 70% or less or about 75% or less of a composition of the present disclosure is added to an isothermal amplification process, e.g., to reduce the concentration of trehalose provided by the composition to a concentration of about 1.5% to about 6% w/v of trehalose, e.g., about 1.5% to about 4% trehalose.

In certain embodiments, a reagent composition, e.g., a reagent composition including one or more non-protein components, can include a concentration of about 5% to about 30% w/v of trehalose that is diluted to a concentration of about 2% to about 6% w/v of trehalose, e.g., when added to an isothermal amplification process. In certain embodiments, a reagent composition including one or more non-protein components, can include a concentration of about 10% to about 30% w/v, e.g., about 15% to about 30% w/v, of trehalose that is reduced to a concentration of about 2% to about 6% w/v of trehalose, e.g., when added to an isothermal amplification process. In certain embodiments, a reagent composition, e.g., a reagent composition including one or more non-protein components, can include a concentration of about 5% to about 25% w/v of trehalose that is diluted to a concentration of about 2% to about 6% w/v of trehalose, e.g., when added to an isothermal amplification process. In certain embodiments, a reagent composition, e.g., a reagent composition including one or more non-protein components, can include a concentration of about 5% to about 20% w/v of trehalose that is diluted to a concentration of about 2% to about 6% w/v of trehalose, e.g., when added to an isothermal amplification process. In certain embodiments, a reagent composition, e.g., a reagent composition including one or more non-protein components, can include a concentration of about 5% to about 15% w/v of trehalose that is diluted to a concentration of about 2% to about 6% w/v of trehalose, e.g., when added to an isothermal amplification process. In certain embodiments, a reagent composition, e.g., a reagent composition including one or more non-protein components, can include a concentration of about 5% to about 10% w/v of trehalose that is diluted to a concentration of about 2% to about 6% w/v of trehalose, e.g., when added to an isothermal amplification process.

In certain embodiments, the present disclosure provides a composition comprising about 5% to about 15% w/v of trehalose, e.g., about 5% to about 7% of trehalose. In certain embodiments, the present disclosure provides a composition comprising about 5.9% w/v of trehalose.

In certain embodiments, the present disclosure provides a composition comprising about 5% to about 20% w/v of trehalose, e.g., about 10% to about 20% of trehalose. In certain embodiments, the present disclosure provides a composition comprising about 15% w/v of trehalose.

In certain embodiments, the present disclosure provides a composition comprising about 7% to about 10% w/v of trehalose, e.g., about 7% to about 10% of trehalose. In certain embodiments, the present disclosure provides a composition comprising about 8.15% w/v of trehalose.

In certain embodiments, the present disclosure provides a composition comprising about 5% to about 6% w/v of trehalose, e.g., about 5.5% to about 6% of trehalose. In certain embodiments, the present disclosure provides a composition comprising about 5.7% w/v of trehalose.

In certain embodiments, the present disclosure provides a composition comprising about 3% to about 5% w/v of trehalose, e.g., about 3.5% to about 4.5% of trehalose. In certain embodiments, the present disclosure provides a composition comprising about 4% w/v of trehalose.

In certain embodiments, the present disclosure provides a composition comprising about 1% to about 2% w/v of trehalose, e.g., about 1.5% to about 2% of trehalose. In certain embodiments, the present disclosure provides a composition comprising about 1.95% w/v of trehalose.

In certain embodiments, the present disclosure provides a composition comprising about 2% to about 3% w/v of trehalose, e.g., about 2.5% to about 3% of trehalose. In certain embodiments, the present disclosure provides a composition comprising about 2.75% w/v of trehalose.

In certain embodiments, a composition of the present disclosure, e.g., reagent composition, further includes one or more of the following (in any combination): at least one recombinase, at least one single-stranded DNA binding protein, at least one DNA polymerase, dNTPs, a buffer, a reducing agent, ATP or an ATP analog, at least one recombinase loading protein, a first primer and, optionally, a second primer, a probe, a nuclease (e.g., an exonuclease), a creatine kinase, a reverse transcriptase and a template nucleic acid molecule, e.g., a single-stranded (e.g., RNA) or double stranded nucleic acid.

In certain embodiments, a composition of the present disclosure, e.g., reagent composition, further includes non-protein components that are present in an isothermal reaction. In certain embodiments, a composition of the present disclosure, e.g., reagent composition, further includes dNTPs, a buffer, salts and/or ATP or an ATP analog.

In certain embodiments, a composition of the present disclosure, e.g., reagent composition, further comprises non-protein components including, but not limited to, dNTPs, a buffer, ATP, salts and phosphocreatine. In certain embodiments, a composition of the present disclosure, e.g., reagent composition, further comprises dNTPs, a buffer (e.g., a Tris buffer), ATP, salts (e.g., potassium acetate) and phosphocreatine.

In certain embodiments, a composition of the present disclosure, e.g., reagent composition, comprises dNTPs, a buffer (e.g., a Tris buffer), ATP, salts (e.g., potassium acetate), phosphocreatine, trehalose and PEG, where trehalose and PEG are present in the composition at a wt % ratio of about 0.79 or less, e.g., about 0.75 or less. In certain embodiments, a composition of the present disclosure, e.g., reagent composition, comprises dNTPs, a buffer (e.g., a Tris buffer), ATP, salts (e.g., potassium acetate), phosphocreatine, trehalose and PEG, where trehalose and PEG are present in the composition at a wt % ratio of about 0.79 or less, e.g., about 0.75 or less, and where trehalose is present in a concentration of about 5% to about 20% w/v (e.g., about 5% to about 15% w/v) and PEG is present in a concentration of about 10% to about 30% w/v (about 15% to about 25% w/v).

In certain embodiments, a composition of the present disclosure, e.g., reagent composition, comprises dNTPs, a buffer (e.g., a Tris buffer), ATP, salts (e.g., potassium acetate), phosphocreatine, trehalose and PEG, where trehalose and PEG are present in the composition at a wt % ratio of about 0.3 to about 0.79, e.g., about 0.3 to about 0.75. In certain embodiments, a composition of the present disclosure, e.g., reagent composition, comprises dNTPs, a buffer (e.g., a Tris buffer), ATP, salts (e.g., potassium acetate), phosphocreatine, trehalose and PEG, where trehalose and PEG are present in the composition at a wt % ratio of about 0.3 to about 0.79, e.g., about 0.3 to about 0.75, and where trehalose is present in a concentration of about 5% to about 20% w/v (e.g., about 5% to about 15% w/v) and PEG is present in a concentration of about 10% to about 30% w/v (e.g., about 15% to about 25% w/v).

In certain embodiments, a composition of the present disclosure, e.g., reagent composition, further includes a recombinase, a single-stranded binding protein, a polymerase, dNTPs, ATP and a primer. In certain embodiments, a composition of the present disclosure, e.g., reagent composition, further includes a recombinase, a single-stranded binding protein, a polymerase, dNTPs, ATP, a primer and a template nucleic acid. In certain embodiments, a composition of the present disclosure does not include a reverse transcriptase.

In certain embodiments, the present disclosure further provides an additional reagent composition (e.g., a second reagent composition) that includes the protein components (PC) (i.e., enzymes) required for an RPA process (e.g., a DNA polymerase, a recombinase, a recombinase loading protein, a single stranded binding protein, creatine kinase, a nuclease (e.g., an exonuclease) and/or a reverse transcriptase). In certain embodiments, the present disclosure further provides an additional reagent composition (e.g., a second reagent composition or third reagent composition that includes the oligonucleotide components (OC) required for an RPA process (e.g., one or more primers and/or one or more probes specific to a target nucleic acid or two or more target nucleic acids). In certain embodiments, trehalose can also be present in the reagent composition including the oligonucleotide components (OC). In certain embodiments, trehalose can also be present in the reagent composition including the protein components (PC). In certain embodiments, PEG is only present in the reagent composition including the non-protein components (NPC).

In certain embodiments, a method of the present disclosure, e.g., an isothermal amplification method of the present disclosure, includes preparing a reagent composition using a composition of the present disclosure, e.g., a composition comprising trehalose and a polyethylene glycol (PEG), where the trehalose and PEG are present in the composition at a trehalose to PEG weight percent (wt %) ratio of about 0.79 or less (e.g. the composition has a trehalose to PEG weight percent (wt %) ratio of about 0.3 to about 0.75), and contacting the target nucleic acid with the reagent composition to amplify the target nucleic acid. In certain embodiments, the method can further include preparing the reagent composition using one or more additional compositions. In certain embodiments, the one or more additional compositions can include the protein components (e.g., a DNA polymerase, a recombinase, a recombinase loading protein, a single stranded binding protein, creatine kinase, a nuclease (e.g., an exonuclease) and/or a reverse transcriptase) and/or the oligonucleotide components (e.g., one or more primers and/or one or more probes specific to a target nucleic acid or two or more target nucleic acids). In certain embodiments, the one or more additional compositions can further include trehalose. For example, but not by way of limitation, an isothermal amplification method of the present disclosure includes (a) preparing a reagent composition using (i) a composition of the present disclosure, e.g., a composition comprising trehalose and a polyethylene glycol (PEG), where the trehalose and PEG are present in the composition at a trehalose to PEG weight percent (wt %) ratio of about 0.79 or less (e.g. the composition has a trehalose to PEG weight percent (wt %) ratio of about 0.3 to about 0.75), (ii) a second composition comprising one or more protein compositions and (iii) a third composition comprising one or more oligonucleotide components and (b) contacting the target nucleic acid with the reagent composition to amplify the target nucleic acid. In certain embodiments, the method can further include contacting the reagent composition and the target nucleic acid with an activator. In certain embodiments, the second composition further includes trehalose. In certain embodiments, the third composition further includes trehalose.

In certain embodiments, a method of the present disclosure, e.g., an isothermal amplification method of the present disclosure, includes providing a reagent composition, where the reagent composition is prepared using a composition comprising trehalose and a polyethylene glycol (PEG), where the trehalose and PEG are present in the composition at a trehalose to PEG weight percent (wt %) ratio of about 0.79 or less (e.g. the composition has a trehalose to PEG weight percent (wt %) ratio of about 0.3 to about 0.75) and (ii) contacting the target nucleic acid with the reagent composition to amplify the target nucleic acid. In certain embodiments, the reagent composition can be further prepared using one or more additional compositions. In certain embodiments, the one or more additional compositions can include the protein components (e.g., a DNA polymerase, a recombinase, a recombinase loading protein, a single stranded binding protein, creatine kinase, a nuclease (e.g., an exonuclease) and/or a reverse transcriptase) and/or the oligonucleotide components (e.g., one or more primers and/or one or more probes specific to a target nucleic acid or two or more target nucleic acids). For example, but not by way of limitation, an isothermal amplification method of the present disclosure includes (a) providing a reagent composition, wherein the reagent composition is prepared using (i) a composition of the present disclosure, e.g., a composition comprising trehalose and a polyethylene glycol (PEG), where the trehalose and PEG are present in the composition at a trehalose to PEG weight percent (wt %) ratio of about 0.79 or less (e.g. the composition has a trehalose to PEG weight percent (wt %) ratio of about 0.3 to about 0.75), (ii) a second composition comprising one or more protein compositions and (iii) a third composition comprising one or more oligonucleotide components and (b) contacting the target nucleic acid with the reagent composition to amplify the target nucleic acid. In certain embodiments, the second composition further includes trehalose. In certain embodiments, the third composition further includes trehalose. In certain embodiments, the method can further include contacting the reagent composition and the target nucleic acid with an activator.

### IV. METHODS OF USE

In certain embodiments, the methods of the present disclosure can be used to detect the presence or absence of a target nucleic acid in a sample. In certain embodiments, the methods of the present disclosure can be used to quantify the amount of a target nucleic acid in a sample.

In certain embodiments, the methods of the present disclosure can be used to amplify and detect target nucleic acids that are associated with a disease or disorder, e.g., to determine if a subject has the disease or disorder. In certain embodiments, the methods of the present disclosure can be used to amplify and detect target nucleic acids that are markers for a disease or disorder. For example, but not by way of limitation, the present disclosure can be used to determine if a subject has a disease or disorder or is at risk of developing the disease or disorder, e.g., cancer. In certain embodiments, the methods of the present disclosure can be used to determine if a subject has a genetic disorder, e.g., by determining if the subject has a genetic mutation associated with the genetic disorder. In certain embodiments, the methods of the present disclosure can be used to amplify and detect target nucleic acids that are derived from a pathogen, e.g., to determine if a subject is infected with the pathogen.

In certain embodiments, the present disclosure can be used to determine the blood type and/or blood group of a subject.

In certain embodiments, the methods of the present disclosure can be used for the screening of blood samples. In certain embodiments, the methods of the present disclosure can be used for the screening of samples derived from a single individual as well as from a plurality of individuals. In certain embodiments, the screening of blood samples can find use in connection with donations of a material, e.g., plasma, platelets, red cells and whole blood. In certain embodiments, the blood sample screened is a whole blood sample. In certain embodiments, the blood sample screened is a lysed whole blood sample. In certain embodiments, the blood sample screened is a serum sample. In certain embodiments, the blood sample screened is a plasma sample.

In certain embodiments, the target nucleic acid is a bacterial, eukaryotic or viral nucleic acid. In certain embodiments, the target nucleic acid is a bacterial nucleic acid. In certain embodiments, the target nucleic acid is a eukaryotic nucleic acid. In certain embodiments, the target nucleic acid is a viral nucleic acid.

In certain embodiments, the target nucleic acid is a nucleic acid derived from SARS-COV-2 (COVID-19), coronaviruses, HIV (e.g., HIV-1 and/or HIV-2), Hepatitis B (HBV), Hepatitis C (HCV), Hepatitis A (HAV), Hepatitis E (HEV), Cytomegalovirus (CMV), Parvovirus B19, Creutzfeldt-Jakob disease (vCJD), Chlamydia, Gonorrhea, West Nile virus (WNV), Zika virus (ZIKV), Dengue, Chikungunya, Influenza (e.g., Influenza A virus, Influenza B virus, or Influenza C virus), Babesia, Plasmodium, Rubella, Varicella-zoster, Herpes Simplex, Polio, syphilis, Smallpox, Vaccinia, Rabies, human T-lymphotropic virus (HTLV), Usutu Virus or Epstein Barr Virus. In certain embodiments, the target nucleic acid is selected from the group consisting of Hepatitis B (HBV), Hepatitis C (HCV), Hepatitis A (HAV), Hepatitis E (HEV) and a combination thereof. In certain embodiments, the target nucleic acid is a nucleic acid derived from HCV. In certain embodiments, the target nucleic acid is a nucleic acid derived from HIV, e.g., HIV-1 and/or HIV-2. In certain embodiments, the target nucleic acid is a nucleic acid derived from HBV. In certain embodiments, the target nucleic acid is a nucleic acid derived from Dengue. In certain embodiments, the target nucleic acid is a nucleic acid derived from Chikungunya.

In certain embodiments, the target nucleic acid is a nucleic acid derived from one or more new or emerging pathogens, viruses and/or agents.

In certain embodiments, if the virus is an RNA-based virus, e.g., HIV-1 and HCV, the nucleic acids to be detected will be RNA. If the virus is a DNA-based virus, e.g., HBV, the nucleic acids to be detected will be DNA. In certain embodiments, the methods can detect ribosomal RNA of the parasite Babesia.

In certain embodiments, the methods of the present disclosure can be used for the detection (e.g., presence or absence) or the quantification of two or more target nucleic acids in a sample, e.g., a plurality of target nucleic acids, by multiplexing. In certain embodiments, higher orders of multiplex amplifications can be employed in connection with the methods of the present disclosure, such that the presence of 3, 4, 5, 6, 7, 8, 9, 10 or more target nucleic acids can be detected in a single sample. In certain embodiments, each target nucleic acid is derived from a different pathogen, infectious agent, gene or mRNA. As used herein, “multiplex analysis” refers to concurrent screening for two or more target nucleic acids, e.g., where each target nucleic acid is derived from a pathogen or infectious agent. As used herein, “multiplex analysis” encompasses concurrent screening of two or more target nucleic acids in a single reaction vessel, e.g., an amplification vessel, as well as screening in separate reaction vessels of two or more target nucleic acids, e.g., where a sample eluate has been split into two more separate reaction vessels, e.g., amplification vessels.

In certain embodiments, the methods of the present disclosure can be used for multiplex analysis of HIV-1, HIV-2, HCV and/or HBV. In certain embodiments, the methods of the present disclosure can be used for multiplex analysis of HIV-1 and HIV-2. In certain embodiments, the methods of the present disclosure can be used for multiplex analysis of HIV-1, HIV-2 and HCV. In certain embodiments, the methods of the present disclosure can be used for multiplex analysis of HIV-1, HIV-2 and HBV. In certain embodiments, the methods of the present disclosure can be used for multiplex analysis of HCV and HBV. In certain embodiments, the methods of the present disclosure can be used for multiplex analysis of HIV-1, HIV-2, HCV and HBV.

In certain embodiments, the methods of the present disclosure can be used for multiplex analysis of Zika Virus, WNV, Chikungunya Virus and/or Dengue Virus. In certain embodiments, the methods of the present disclosure can be used for multiplex analysis of Zika Virus and WNV. In certain embodiments, the methods of the present disclosure can be used for multiplex analysis of Chikungunya Virus and Dengue Virus. In certain embodiments, the methods of the present disclosure can be used for multiplex analysis of Chikungunya Virus and WNV. In certain embodiments, the methods of the present disclosure can be used for multiplex analysis of Zika Virus and Dengue.

In certain embodiments, the methods of the present disclosure can be used for multiplex analysis of Babesia and Malaria.

In certain embodiments, the methods of the present disclosure can be used for multiplex analysis of Parvovirus B19 and HAV.

In certain embodiments, a method of the present disclosure, e.g., an isothermal amplification method of the present disclosure, includes (i) providing a reagent composition, where the reagent composition is prepared using a composition comprising trehalose and a polyethylene glycol (PEG), where the trehalose and PEG are present in the composition at a trehalose to PEG weight percent (wt %) ratio of about 0.75 or less (e.g. the composition has a trehalose to PEG weight percent (wt %) ratio of about 0.3 to about 0.75 or about 0.3 to about 0.65) and (ii) contacting a sample comprising the target nucleic acid with the reagent composition to amplify the target nucleic acid. In certain embodiments, the target nucleic acid is derived from one or more of HIV-1, HIV-2, HCV and HBV. In certain embodiments, the isothermal amplification method is a multiplex reaction, and the target nucleic acid is derived from HIV-1, HIV-2, HCV and HBV. In certain embodiments, the composition can include from about 10% to about 20% w/v of trehalose, e.g., about 15% w/v of trehalose, and about 15% to about 25% w/v of PEG. In certain embodiments, the composition can further include dNTPs, a buffer, salts and/or ATP or an ATP analog.

In certain embodiments, a method of the present disclosure, e.g., an isothermal amplification method of the present disclosure, includes (i) providing a reagent composition, where the reagent composition is prepared using a composition comprising trehalose and a polyethylene glycol (PEG), where the trehalose and PEG are present in the composition at a trehalose to PEG weight percent (wt %) ratio of about 0.75 or less (e.g. the composition has a trehalose to PEG weight percent (wt %) ratio of about 0.3 to about 0.60 or about 0.3 to about 0.50) and (ii) contacting a sample comprising the target nucleic acid with the reagent composition to amplify the target nucleic acid. In certain embodiments, the target nucleic acid is derived from the Zika virus. In certain embodiments, the composition can include from about 5% to about 10% w/v of trehalose and about 15% to about 25% w/v of PEG. In certain embodiments, the composition can further include dNTPs, a buffer, salts and/or ATP or an ATP analog.

In certain embodiments, a method of the present disclosure, e.g., an isothermal amplification method of the present disclosure, includes (i) providing a reagent composition, where the reagent composition is prepared using a composition comprising trehalose and a polyethylene glycol (PEG), where the trehalose and PEG are present in the composition at a trehalose to PEG weight percent (wt %) ratio of about 0.75 or less (e.g. the composition has a trehalose to PEG weight percent (wt %) ratio of about 0.3 to about 0.50 or about 0.3 to about 0.40) and (ii) contacting a sample comprising the target nucleic acid with the reagent composition to amplify the target nucleic acid. In certain embodiments, the target nucleic acid is derived from the Dengue virus. In certain embodiments, the target nucleic acid is derived from the Chikungunya virus. In certain embodiments, the target nucleic acid is derived from the Chikungunya virus and the Dengue virus. In certain embodiments, the composition can include from about 5% to about 10% w/v of trehalose and about 15% to about 25% w/v of PEG. In certain embodiments, the composition can further include dNTPs, a buffer, salts and/or ATP or an ATP analog.

### V. KITS

The present disclosure further provides kits for performing the methods of the present disclosure. In certain embodiments, the present disclosure provides kits containing materials for performing a method of the present disclosure. In certain embodiments, a kit of the present disclosure includes a container containing reagents, e.g., a reagent composition, for performing a method of the present disclosure that comprises trehalose and PEG, e.g., at the ratios and amounts described herein.

In certain embodiments, the kit contains a reagent composition that includes trehalose and PEG at a wt % ratio of about 0.3 to about 0.79. In certain embodiments, the kit contains a reagent composition that include trehalose and PEG at a wt % ratio of about 0.45 to about 0.75, e.g., about 0.5 to about 0.7, about 0.45 to about 0.65 or about 0.5 to about 0.55. In certain embodiments, the kit contains a reagent composition that include trehalose and PEG at a wt % ratio of about 0.3 to about 0.45, e.g., about 0.3 to about 0.4, about 0.3 to about 0.35 or about 0.4 to about 0.45. In certain embodiments, the kit contains a reagent composition that includes trehalose and PEG at a wt % ratio of about 0.79 or less or about 0.75 or less, e.g., 0.65 or less, 0.55 or less, 0.45 or less or 0.35 or less. In certain embodiments, the kit contains a reagent composition that includes trehalose and PEG at a wt % ratio of about 0.65 or less. In certain embodiments, the kit contains a reagent composition that includes trehalose and PEG at a wt % ratio of about 0.55 or less. In certain embodiments, the kit contains a reagent composition that includes trehalose and PEG at a wt % ratio of about 0.45 or less. In certain embodiments, the kit contains a reagent composition that includes trehalose and PEG at a wt % ratio of about 0.35 or less.

In certain embodiments, a kit of the present disclosure provides a reagent composition that when added to an isothermal amplification process (e.g., diluted) results in a concentration of about 2% to about 8.5% w/v of PEG, e.g., about 5% to about 8% w/v of PEG, about 6% to about 8% w/v of PEG, about 7% to about 8% w/v of PEG or about 8% to about 8.5% w/v of PEG. In certain embodiments, the kit contains a reagent composition that when added to an isothermal amplification process (e.g., diluted) results in a concentration of about 2% to about 8.5% w/v of PEG, e.g., about 5% to about 8% w/v of PEG, about 6% to about 8% w/v of PEG, about 7% to about 8% w/v of PEG or about 8% to about 8.5% w/v of PEG. In certain embodiments, the kit contains a reagent composition that when added to an isothermal amplification process (e.g., diluted) results in a concentration of about 5% to about 8.5% w/v of PEG. In certain embodiments, the kit contains a reagent composition that when added to an isothermal amplification process (e.g., diluted) results in a concentration of about 6.3% of PEG. In certain embodiments, the kit contains a reagent composition that when added to an isothermal amplification process (e.g., diluted) results in a concentration of about 6.5% w/v of PEG. In certain embodiments, the kit contains a reagent composition that when added to an isothermal amplification process (e.g., diluted) results in a concentration of about 7% of PEG. In certain embodiments, the kit contains a reagent composition that when added to an isothermal amplification process (e.g., diluted) results in a concentration of about 7.4% of PEG. In certain embodiments, the kit contains a reagent composition that when added to an isothermal amplification process (e.g., diluted) results in a concentration of about 8.4% of PEG. For example, but not by way of limitation, when about 5%, about 10%, about 15%, about 20%, about 25%, about 30%, about 35%, about 40%, about 45%, about 50%, about 55%, about 60%, about 65%, about 70% or about 75% of a reagent composition from a kit of the present disclosure is added to an isothermal amplification process it results in a reaction concentration of about 5% to about 8.5% w/v of PEG. In certain embodiments, when about 5% or less, about 10% or less, about 15% or less, about 20% or less, about 25% or less, about 30% or less, about 35% or less, about 40% or less, about 45% or less, about 50% or less, about 55% or less, about 60% or less, about 65% or less, about 70% or less or about 75% or less of a reagent composition from a kit of the present disclosure is added to an isothermal amplification process it results in a reaction concentration of about 5% to about 8.5% w/v of PEG. For example, but not by way of limitation, the reagent composition can include from about 10% to about 30% w/v of PEG to result in a reaction concentration of about 5% to about 8.5% w/v of PEG. In certain embodiments, the present disclosure provides a kit comprising reagent composition comprising about 10% to about 30% w/v of PEG. In certain embodiments, the present disclosure provides a kit comprising reagent composition comprising about 10% to about 25% w/v of PEG, e.g., about 30% w/v of PEG. In certain embodiments, the present disclosure provides a kit comprising reagent composition comprising about 10% to about 20% w/v of PEG.

In certain embodiments, the kit contains a reagent composition that includes about 2% to about 8.5% w/v of PEG, e.g., about 5% to about 8% w/v of PEG, about 6% to about 8% w/v of PEG, about 7% to about 8% w/v of PEG, about 8% to about 8.5% w/v of PEG, about 6% to about 7% w/v of PEG or about 5% to about 7% w/v of PEG. In certain embodiments, the kit contains a reagent composition that includes about 5.5% w/v of PEG. In certain embodiments, the kit contains a reagent composition that includes about 6.3% w/v of PEG. In certain embodiments, the kit contains a reagent composition that includes about 6.5% w/v of PEG. In certain embodiments, the kit contains a reagent composition that includes about 7.4% w/v of PEG. In certain embodiments, the kit contains a reagent composition that includes about 7.7% w/v of PEG. In certain embodiments, the kit contains a reagent composition that includes about 8.4% w/v of PEG. In certain embodiments, the PEG has a molecular weight between about 30,000 Daltons to about 40,000 daltons, e.g., about 35,000 daltons.

In certain embodiments, the kit contains a reagent composition that when added to an isothermal amplification process (e.g., diluted) results in a concentration of about 2% to about 6% w/v of trehalose. For example, but not by way of limitation, when about 5%, about 10%, about 15%, about 20%, about 25%, about 30%, about 35%, about 40%, about 45%, about 50%, about 55%, about 60%, about 65%, about 70% or about 75% of a reagent composition from a kit of the present disclosure is added to an isothermal amplification process it reduces the concentration of the trehalose provided by the composition to a concentration of about 2% to about 6% w/v of trehalose, e.g., about 2% to about 4% trehalose. In certain embodiments, when about 5% or less, about 10% or less, about 15% or less, about 20% or less, about 25% or less, about 30% or less, about 35% or less, about 40% or less, about 45% or less, about 50% or less, about 55% or less, about 60% or less, about 65% or less, about 70% or less or about 75% or less of a reagent composition from a kit of the present disclosure is added to an isothermal amplification process it reduces the concentration of the trehalose provided by the composition to a concentration of about 2% to about 6% w/v of trehalose, e.g., about 2% to about 4% trehalose. For example, but not by way of limitation, the reagent composition can include from about 10% to about 30% w/v, e.g., about 15% to about 30% w/v, of trehalose that is reduced to a concentration of about 2% to about 6% w/v of trehalose, e.g., when added to an isothermal amplification process. In certain embodiments, the present disclosure provides a kit comprising a reagent composition comprising about 5% to about 15% w/v of trehalose, e.g., about 5% to about 7% of trehalose. In certain embodiments, the present disclosure provides a kit comprising a reagent composition comprising about 5.9% w/v of trehalose. In certain embodiments, the present disclosure provides a kit comprising a reagent composition comprising about 5% to about 20% w/v of trehalose, e.g., about 10% to about 20% of trehalose. In certain embodiments, the present disclosure provides a kit comprising a reagent composition comprising about 15% w/v of trehalose. In certain embodiments, the present disclosure provides a reagent composition comprising about 7% to about 10% w/v of trehalose, e.g., about 7% to about 10% of trehalose. In certain embodiments, the present disclosure provides a kit comprising a reagent composition comprising about 8.15% w/v of trehalose. In certain embodiments, the present disclosure provides a kit comprising a reagent composition comprising about 5% to about 6% w/v of trehalose, e.g., about 5.5% to about 6% of trehalose. In certain embodiments, the present disclosure provides a kit comprising a reagent composition comprising about 5.7% w/v of trehalose.

In certain embodiments, the kit contains a reagent composition that comprises about 3% to about 5% w/v of trehalose, e.g., about 3.5% w/v to about 4.5% w/v of trehalose. In certain embodiments, the kit contains a reagent composition that comprises about 4% w/v of trehalose.

In certain embodiments, the kit contains a reagent composition that comprises about 2% to about 3% w/v of trehalose, e.g., about 2.5% w/v to about 3% w/v of trehalose. In certain embodiments, the kit contains a reagent composition that comprises about 2.75% w/v of trehalose.

In certain embodiments, the kit contains a reagent composition that comprises about 1% to about 2% w/v of trehalose, e.g., about 1.5% w/v to about 2% w/v of trehalose. In certain embodiments, the kit contains a reagent composition that comprises about 1.95% w/v of trehalose.

In certain embodiments, the present disclosure provides a kit comprising a reagent composition comprising about 10% to about 20% w/v of PEG and about 5% to about 20% w/v of trehalose. In certain embodiments, the present disclosure provides a kit comprising a reagent composition comprising about 15% to about 20% w/v of PEG and about 8% to about 20% w/v of trehalose.

In certain embodiments, the present disclosure provides a kit comprising a reagent composition comprising about 5% to about 10% w/v of PEG and about 6% to about 12% w/v of trehalose. In certain embodiments, the present disclosure provides a kit comprising a reagent composition comprising about 6% to about 10% w/v of PEG and about 6% to about 12% w/v of trehalose.

In certain embodiments, the reagent composition can further include one or more of the following components (in any combination) and/or a kit of the present disclosure can include one or more additional reagent compositions that include one or more of the following components (in any combination): at least one recombinase, at least one single-stranded DNA binding protein, at least one DNA polymerase, dNTPs, a buffer, a reducing agent, ATP or an ATP analog, at least one recombinase loading protein, a first primer and, optionally, a second primer, a probe, a reverse transcriptase, a creatine kinase, a nuclease and a template nucleic acid molecule, e.g., a single-stranded (e.g., RNA) or double stranded nucleic acid. In certain embodiments, a composition of the present disclosure further includes dNTPs, a buffer, salts, ATP or an ATP analog and/or a crowding agent.

In certain embodiments, a composition of the present disclosure further includes dNTPs, a buffer, salts, ATP and/or an ATP analog. In certain embodiments, a kit of the present disclosure can include a reagent composition that further comprises non-protein components including, but not limited to, dNTPs, a buffer, ATP, salts and phosphocreatine. In certain embodiments, a kit of the present disclosure can include a reagent composition that further comprises dNTPs, a buffer (e.g., a Tris buffer), ATP, salts (e.g., potassium acetate) and phosphocreatine. In certain embodiments, a composition of the present disclosure further includes a recombinase, a single-stranded binding protein, a polymerase, dNTPs, ATP, a primer and a template nucleic acid. In certain embodiments, a composition of the present disclosure does not include a reverse transcriptase. Alternatively, such reagents can be present in a different container than the trehalose and PEG. For example, but not by way of limitation, a kit of the present disclosure can include a second composition (e.g., second reagent composition in a second container) that includes the enzymes used in an isothermal reaction including, but not limited to, at least one recombinase (e.g., UvsX), at least one single-stranded DNA binding protein (e.g., gp32), at least one DNA polymerase (e.g., Pol I), at least one recombinase loading protein (e.g., UvsY), an exonuclease (e.g., Exonuclease III), creatine kinase and/or at least one reverse transcriptase (e.g., EIAV-RT). In certain embodiments, the second reagent composition does not include a reverse transcriptase. In certain embodiments, a kit of the present disclosure can include a third composition (e.g., third reagent composition in a third container) that includes one or more primers (e.g., a first primer and a second primer) and one/or more probes. In certain embodiments, a kit of the present disclosure can include a fourth composition (e.g., fourth reagent composition in a fourth container) that includes an activator, e.g., MgOAc. In certain embodiments, trehalose can also be present in the reagent composition including the enzymes. In certain embodiments, trehalose can also be present in the reagent composition including the one or more primers (e.g., a first primer and a second primer) and one/or more probes.

In certain embodiments, a kit of the present disclosure can include a reagent composition (e.g., in a first container) that comprises dNTPs, a buffer (e.g., a Tris buffer), ATP, salts (e.g., potassium acetate), phosphocreatine, trehalose and PEG, where trehalose and PEG are present in the composition at a wt % ratio of about 0.79 or less. In certain embodiments, a kit of the present disclosure can include a reagent composition (e.g., in a first container) that comprises dNTPs, a buffer (e.g., a Tris buffer), ATP, salts (e.g., potassium acetate), phosphocreatine, trehalose and PEG, where trehalose and PEG are present in the composition at a wt % ratio of about 0.70 or less. In certain embodiments, a kit of the present disclosure can include a reagent composition (e.g., in a first container) that comprises dNTPs, a buffer (e.g., a Tris buffer), ATP, salts (e.g., potassium acetate), phosphocreatine, trehalose and PEG, where trehalose and PEG are present in the composition at a wt % ratio of about 0.6 or less. In certain embodiments, a kit of the present disclosure can include a reagent composition (e.g., in a first container) that comprises dNTPs, a buffer (e.g., a Tris buffer), ATP, salts (e.g., potassium acetate), phosphocreatine, trehalose and PEG, where trehalose and PEG are present in the composition at a wt % ratio of about 0.45 or less. In certain embodiments, a kit of the present disclosure can include a reagent composition (e.g., in a first container) that comprises dNTPs, a buffer (e.g., a Tris buffer), ATP, salts (e.g., potassium acetate), phosphocreatine, trehalose and PEG, where trehalose and PEG are present in the composition at a wt % ratio of about 0.35 or less.

In certain embodiments, a kit of the present disclosure can include a reagent composition (e.g., in a first container) that comprises dNTPs, a buffer (e.g., a Tris buffer), ATP, salts (e.g., potassium acetate), phosphocreatine, trehalose and PEG, where trehalose and PEG are present in the composition at a wt % ratio of about 0.3 to about 0.79, e.g., about 0.3 to about 0.45, about 0.45 to about 0.65, about 0.5 to about 0.55, about 0.65 to about 0.70 or about 0.7 to 0.79. In certain embodiments, a kit of the present disclosure can include a reagent composition (e.g., in a first container) that consists essentially of dNTPs, a buffer (e.g., a Tris buffer), ATP, salts (e.g., potassium acetate), phosphocreatine, trehalose and PEG, where trehalose and PEG are present in the composition at a wt % ratio of about 0.3 to about 0.79. In certain embodiments, a kit of the present disclosure can include a reagent composition (e.g., in a first container) that consists of dNTPs, a buffer (e.g., a Tris buffer), ATP, salts (e.g., potassium acetate), phosphocreatine, trehalose and PEG, where trehalose and PEG are present in the composition at a wt % ratio of about 0.3 to about 0.79.

In certain embodiments, the presently disclosed reagents and/or compositions can be lyophilized.

Examples of suitable containers include, but are not limited to, bottles, test tubes, vials and microtiter plates. The containers can be formed from a variety of materials such as glass or plastic.

In certain embodiments, the kit further includes a package insert that provides instructions for using the components provided in the kit. For example, a kit of the present disclosure can include a package insert that provides instructions for performing methods of the present disclosure.

In certain embodiments, the kit can include other materials desirable from a commercial and user standpoint, including other buffers and diluents.

### VI. SYSTEMS

The present disclosure further provides systems for performing the methods of the present disclosure. In certain embodiments, the system is an automatic system. In certain embodiments, the automatic system that can be used for performing the methods of the present disclosure can include a sample preparation area (e.g., an area for isolating nucleic acids from a sample), a nucleic acid amplification area and a nucleic acid detection area. In certain embodiments, the nucleic acid amplification area and the nucleic acid detection area are the same. An automated system for performing the methods of the present disclosure is provided in International Patent Application PCT/US2022/027067, which is incorporated herein by reference in its entirety. For example, but not by way of limitation, the system of FIGS. 68A-68D of PCT/US2022/027067 can be used to perform the methods of the present disclosure.

In certain embodiments, a system of the present disclosure includes containers and/or reservoirs that includes one or more compositions disclosed herein for performing an isothermal amplification reaction (e.g., an RPA process). In certain embodiments, a system of the present disclosure can include at least one container or reservoir that includes a composition (e.g., reagent composition) that includes trehalose and PEG at the ratios and amounts described herein.

In certain embodiments, a system of the present disclosure can include at least one container or reservoir that includes a composition (e.g., reagent composition) comprising trehalose and PEG at a wt % ratio of about 0.25 to about 0.79 or about 0.3 to about 0.79, e.g., about 0.3 to about 0.45, about 0.45 to about 0.65, about 0.5 to about 0.55, about 0.65 to about 0.70 or about 0.7 to 0.79. In certain embodiments, the system can include at least one container or reservoir that includes a composition (e.g., reagent composition) comprising trehalose and PEG at a wt % ratio of about 0.45 to about 0.75, e.g., about 0.5 to about 0.7, about 0.45 to about 0.65, about 0.5 to about 0.55 or about 0.7 to about 0.75. In certain embodiments, a system of the present disclosure can include at least one container or reservoir that includes a composition (e.g., reagent composition) that includes trehalose and PEG at a wt % ratio of about 0.79 or less or about 0.75 or less, e.g., 0.65 or less, 0.55 or less, 0.45 or less or 0.35 or less.

In certain embodiments, a system of the present disclosure can include at least one container or reservoir that includes a reagent composition that when added to an isothermal amplification process (e.g., diluted) results in a concentration of about 2% to about 8.5% w/v of PEG, e.g., about 5% to about 8% w/v of PEG, about 6% to about 8% w/v of PEG or about 8% to about 8.5% w/v of PEG. In certain embodiments, the system contains a reagent composition that when added to an isothermal amplification process (e.g., diluted) results in a concentration of about 2% to about 8.5% w/v of PEG, e.g., about 5% to about 8% w/v of PEG, about 6% to about 8% w/v of PEG or about 8% to about 8.5% w/v of PEG. In certain embodiments, the system contains a reagent composition that when added to an isothermal amplification process (e.g., diluted) results in a concentration of about 5% to about 8.5% w/v of PEG. In certain embodiments, the system contains a reagent composition that when added to an isothermal amplification process (e.g., diluted) results in a concentration of about 6.5% w/v of PEG. In certain embodiments, the system contains a reagent composition that when added to an isothermal amplification process (e.g., diluted) results in a concentration of about 7% of PEG. In certain embodiments, the system contains a reagent composition that when added to an isothermal amplification process (e.g., diluted) results in a concentration of about 7.4% of PEG. In certain embodiments, the system contains a reagent composition that when added to an isothermal amplification process (e.g., diluted) results in a concentration of about 8.4% of PEG. For example, but not by way of limitation, when about 5%, about 10%, about 15%, about 20%, about 25%, about 30%, about 35%, about 40%, about 45%, about 50%, about 55%, about 60%, about 65%, about 70% or about 75% of a reagent composition from a system of the present disclosure is added to an isothermal amplification process it results in a reaction concentration of about 5% to about 8.5% w/v of PEG. For example, but not by way of limitation, a system of the present disclosure can include at least one container or reservoir that contains a reagent composition that includes about 10% to about 30% w/v of PEG, e.g., about 15% to about 25% w/v of PEG or about 20% w/v of PEG.

In certain embodiments, the system can include at least one container or reservoir that contains a reagent composition that includes about 2% to about 8.5% w/v of PEG, e.g., about 5% to about 8% w/v of PEG, about 6% to about 8% w/v of PEG, about 7% to about 8% w/v of PEG, about 6% to about 7% w/v of PEG, about 5% to about 7% w/v of PEG or about 8% to about 8.5% w/v of PEG. In certain embodiments, the system contains a reagent composition that includes about 5.5% w/v of PEG. In certain embodiments, the system can include at least one container or reservoir that contains a reagent composition that includes about 6.3% w/v of PEG. In certain embodiments, the system contains a reagent composition that includes about 6.5% w/v of PEG. In certain embodiments, the system can include at least one container or reservoir that contains a reagent composition that includes about 7.7% w/v of PEG. In certain embodiments, the system can include at least one container or reservoir that contains a reagent composition that includes about 7.4% w/v of PEG. In certain embodiments, the system can include at least one container or reservoir that contains a reagent composition that includes about 8.5% w/v of PEG. In certain embodiments, the PEG has a molecular weight between about 30,000 Daltons to about 40,000 daltons, e.g., about 35,000 daltons.

In certain embodiments, the system can include at least one container or reservoir that contains a reagent composition that when added to an isothermal amplification process (e.g., diluted) results in a concentration of about 1.5% to about 6% w/v of trehalose. In certain embodiments, the system can include at least one container or reservoir that contains a reagent composition that when added to an isothermal amplification process (e.g., diluted) results in a concentration of about 2% to about 6% w/v of trehalose. For example, but not by way of limitation, when about 5%, about 10%, about 15%, about 20%, about 25%, about 30%, about 35%, about 40%, about 45%, about 50%, about 55%, about 60%, about 65%, about 70% or about 75% of a reagent composition from a system of the present disclosure is added to an isothermal amplification process it results in a reaction concentration of about 2% to about 6% w/v of trehalose, e.g., about 2% to about 4% trehalose. For example, but not by way of limitation, a system of the present disclosure can include at least one container or reservoir that contains a reagent composition that includes about 5% to about 20% w/v of trehalose, e.g., about 5% to about 15% w/v of trehalose or about 10% to about 15% w/v of trehalose.

In certain embodiments, the system can include at least one container or reservoir that contains a reagent composition that comprises about 3% to about 5% w/v of trehalose, e.g., about 3.5% w/v to about 4.5% w/v of trehalose. In certain embodiments, the system contains a reagent composition that comprises about 4% w/v of trehalose.

In certain embodiments, the system can include at least one container or reservoir that contains a reagent composition that comprises about 1% to about 2% w/v of trehalose, e.g., about 1.5% w/v to about 2% w/v of trehalose. In certain embodiments, the system contains a reagent composition that comprises about 1.95% w/v of trehalose.

In certain embodiments, the system can include at least one container or reservoir that contains a reagent composition that comprises about 2% to about 3% w/v of trehalose, e.g., about 2.5% w/v to about 3% w/v of trehalose. In certain embodiments, the system contains a reagent composition that comprises about 2.75% w/v of trehalose.

In certain embodiments, a system of the present disclosure can include at least one container or reservoir that includes a composition (e.g., reagent composition) that includes about 10% to about 25% w/v of PEG, e.g., about 10% to about 20% w/v of PEG, and about 5% to about 20% w/v of trehalose, e.g., about 5% w/v to about 15% w/v of trehalose.

In certain embodiments, a system of the present disclosure can include at least one container or reservoir that includes a composition (e.g., reagent composition) that includes about 2% to about 8.5% w/v of PEG, e.g., about 5% to about 8.5% w/v of PEG, and about 2% to about 5% w/v of trehalose, e.g., about 1.95% w/v of trehalose or about 4% w/v of trehalose.

In certain embodiments, the reagent composition in a system of the present disclosure can further include one or more of the following components (in any combination) and/or a system of the present disclosure can include one or more additional reagent compositions that include one or more of the following components (in any combination): at least one recombinase, at least one single-stranded DNA binding protein, at least one DNA polymerase, dNTPs, a buffer, a reducing agent, ATP or an ATP analog, at least one recombinase loading protein, a first primer and, optionally, a second primer, a probe, a reverse transcriptase, a creatine kinase, a nuclease and a template nucleic acid molecule, e.g., a single-stranded (e.g., RNA) or double stranded nucleic acid. In certain embodiments, a composition in a system of the present disclosure further includes dNTPs, a buffer, salts, ATP or an ATP analog and/or a crowding agent.

In certain embodiments, a system of the present disclosure can include at least one container or reservoir that includes a composition that further includes dNTPs, a buffer, salts, ATP and/or an ATP analog. In certain embodiments, a system of the present disclosure can include a reagent composition that further comprises non-protein components including, but not limited to, dNTPs, a buffer, ATP, salts and phosphocreatine. In certain embodiments, a system of the present disclosure can include a reagent composition that further comprises dNTPs, a buffer (e.g., a Tris buffer), ATP, salts (e.g., potassium acetate) and phosphocreatine. In certain embodiments, a composition of the present disclosure further includes a recombinase, a single-stranded binding protein, a polymerase, dNTPs, ATP, a primer and a template nucleic acid. In certain embodiments, a composition of the present disclosure does not include a reverse transcriptase. Alternatively, such reagents can be present in a different container than the trehalose and PEG. For example, but not by way of limitation, a system of the present disclosure can include a second composition (e.g., second reagent composition in a second container or reservoir of the system) that includes the enzymes used in an isothermal reaction including, but not limited to, at least one recombinase (e.g., UvsX), at least one single-stranded DNA binding protein (e.g., gp32), at least one DNA polymerase (e.g., Pol I), at least one recombinase loading protein (e.g., UvsY), an exonuclease (e.g., Exonuclease III), creatine kinase and/or at least one reverse transcriptase (e.g., EIAV-RT). In certain embodiments, the second reagent composition does not include a reverse transcriptase. In certain embodiments, a system of the present disclosure can include a third composition (e.g., third reagent composition in a third container or reservoir of the system) that includes one or more primers (e.g., a first primer and a second primer) and one/or more probes. In certain embodiments, a system of the present disclosure can include a fourth composition (e.g., fourth reagent composition in a fourth container or reservoir of the system) that includes an activator, e.g., MgOAc. In certain embodiments, trehalose can also be present in the reagent composition including the enzymes. In certain embodiments, trehalose can also be present in the reagent composition including the one or more primers (e.g., a first primer and a second primer) and one/or more probes.

In certain embodiments, a system of the present disclosure can include at least one container or reservoir that includes a reagent composition (e.g., in a first container or reservoir) that comprises dNTPs, a buffer (e.g., a Tris buffer), ATP, salts (e.g., potassium acetate), phosphocreatine, trehalose and PEG, where trehalose and PEG are present in the composition at a wt % ratio of about 0.79 or less.

In certain embodiments, a system of the present disclosure can include at least one container or reservoir that includes a reagent composition (e.g., in a first container or reservoir) that comprises dNTPs, a buffer (e.g., a Tris buffer), ATP, salts (e.g., potassium acetate), phosphocreatine, trehalose and PEG, where trehalose and PEG are present in the composition at a wt % ratio of about 0.3 to about 0.79, e.g., about 0.3 to about 0.75. In certain embodiments, a system of the present disclosure can include at least one container or reservoir that includes a reagent composition (e.g., in a first container or reservoir) that consists essentially of dNTPs, a buffer (e.g., a Tris buffer), ATP, salts (e.g., potassium acetate), phosphocreatine, trehalose and PEG, where trehalose and PEG are present in the composition at a wt % ratio of about 0.3 to about 0.79, e.g., about 0.3 to about 0.75. In certain embodiments, a system of the present disclosure can include at least one container or reservoir that includes a reagent composition (e.g., in a first container or reservoir) that consists of dNTPs, a buffer (e.g., a Tris buffer), ATP, salts (e.g., potassium acetate), phosphocreatine, trehalose and PEG, where trehalose and PEG are present in the composition at a wt % ratio of about 0.3 to about 0.79, e.g., about 0.3 to about 0.75. In certain embodiments, PEG can be present in the composition at a concentration of about 10% to about 30% w/v and trehalose can be present in the composition at a concentration of about 5% to about 20% w/v.

In certain embodiments, the presently disclosed reagents and/or compositions present in the system can be lyophilized.

Examples of suitable containers or reservoirs include, but are not limited to, bottles, test tubes, vials and microtiter plates. The containers or reservoirs can be formed from a variety of materials such as glass or plastic.

In certain embodiments, the system can include other materials desirable from a commercial and user standpoint, including other buffers and diluents.

### VII. EXEMPLARY EMBODIMENTS

A. The present disclosure provides an isothermal amplification process for amplifying a target nucleic acid, wherein the isothermal amplification process comprises:
   (i) preparing a reagent composition using a composition comprising trehalose and a polyethylene glycol (PEG), wherein the trehalose and PEG are present in the composition at a trehalose to PEG weight percent (wt %) ratio of about 0.79 or less; and
   (ii) contacting the target nucleic acid with the reagent composition to amplify the target nucleic acid.
B. The present disclosure provides an isothermal amplification process for amplifying a target nucleic acid, wherein the isothermal amplification process comprises:
   (i) providing a reagent composition, wherein the reagent composition is prepared using a composition comprising trehalose and a polyethylene glycol (PEG), and wherein the trehalose and PEG are present in the composition at a trehalose to PEG weight percent (wt %) ratio of about 0.79 or less; and
   (ii) contacting the target nucleic acid with the reagent composition to amplify the target nucleic acid.
B1. The isothermal amplification process of A or B, wherein the trehalose to PEG wt % ratio is about 0.25 to about 0.79.
B2. The isothermal amplification process of any one of A-B1, wherein the trehalose to PEG wt % ratio is about 0.25 to about 0.75.
B3. The isothermal amplification process of any one of A-B2, wherein the trehalose to PEG wt % ratio is about 0.30 to about 0.70.
B4. The isothermal amplification process of any one of A-B3, wherein the trehalose to PEG wt % ratio is about 0.30 to about 0.65.
B5. The isothermal amplification process of any one of A-B4, wherein the trehalose to PEG wt % ratio is about 0.30 to about 0.55.
B5-1. The isothermal amplification process of any one of A-B5, wherein the trehalose to PEG wt % ratio is about 0.30 to about 0.50.
B6. The isothermal amplification process of any one of A-B5-1, wherein the PEG is present in the composition in an amount of about 10% to about 30% weight to volume (w/v).
B7. The isothermal amplification process of any one of A-B6, wherein the PEG is present in the composition in an amount of about 15% to about 20% weight to volume (w/v).
B8. The isothermal amplification process of any one of A-B7, wherein the reagent composition comprises PEG at an amount from about 2% to about 8% weight to volume (w/v).
B9. The isothermal amplification process of any one of A-B8, wherein the trehalose is present in the composition in an amount of about 5% to about 20% w/v.
B10. The isothermal amplification process of any one of A-B9, wherein the trehalose is present in the composition in an amount of about 5% to about 15% w/v.
B11. The isothermal amplification process of any one of A-B10, wherein the trehalose is present in the composition in an amount of about 5% to about 12% w/v.
B12. The isothermal amplification process of any one of A-B11, wherein the reagent composition comprises trehalose at an amount of about 3% to about 12% w/v.
B12-1. The isothermal amplification process of any one of A-B12, wherein the reagent composition comprises trehalose at an amount of about 6% to about 12% w/v.
B13. The isothermal amplification process of any one of A-B12, wherein the reagent composition comprises trehalose at an amount of about 3% to about 10% w/v.
B13-1. The isothermal amplification process of any one of A-B13, wherein the reagent composition comprises trehalose at an amount of about 5% to about 10% w/v.
B14. The isothermal amplification process of any one of A-B13-1, wherein the composition further comprises one or more of ATP, dNTPs, phosphocreatine and one or more salts.
B15. The isothermal amplification process of any one of A-B14, wherein the composition further comprises ATP, dNTPs, phosphocreatine and one or more salts.
B16. The isothermal amplification process of any one of A-B15, wherein the reagent composition further comprises one or more of a DNA polymerase, a recombinase loading protein, a single stranded binding protein, ATP, dNTPs or a mixture of dNTPs and ddNTPs, a reducing agent, creatine kinase, a nuclease (e.g., an exonuclease), one or more primers, a nucleic acid probe and a reverse transcriptase.
C. The present disclosure provides an isothermal amplification process for amplifying a target nucleic acid comprising contacting the target nucleic acid with a reagent composition comprising trehalose and a polyethylene glycol (PEG) to amplify the target nucleic acid, wherein the trehalose and PEG are in the reagent composition at a weight percent (wt %) ratio of about 0.79 or less.
D. The present disclosure provides an isothermal amplification process for amplifying a target nucleic acid, wherein the isothermal amplification process is performed in the presence of trehalose and a polyethylene glycol (PEG) at a trehalose to PEG weight percent (wt %) ratio of about 0.79 or less.
D1. The isothermal amplification process of C or D, wherein the trehalose to PEG wt % ratio is about 0.3 to about 0.79.
D2. The isothermal amplification process of any one of C-D1, wherein the trehalose to PEG wt % ratio is about 0.45 to about 0.75.
D3. The isothermal amplification process of any one of C-D2, wherein the trehalose to PEG wt % ratio is about 0.50 to about 0.70.
D4. The isothermal amplification process of any one of C-D3, wherein the trehalose to PEG wt % ratio is about 0.50 to about 0.60.
D5. The isothermal amplification process of any one of C-D4, wherein the PEG is present in the reagent composition in an amount of about 2% to about 30% weight to volume (w/v).
D6. The isothermal amplification process of any one of C-D5, wherein the PEG is present in the isothermal amplification process and/or the reagent composition in an amount of about 2% to about 20% weight to volume (w/v).
D7. The isothermal amplification process of any one of C-D6, wherein the PEG is present in the isothermal amplification process and/or the reagent composition in an amount of about 2% to about 8% weight to volume (w/v).
D8. The isothermal amplification process of any one of C-D7, wherein the trehalose is present in the isothermal amplification process and/or the reagent composition in an amount of about 3% to about 20% w/v.
D9. The isothermal amplification process of any one of C-D8, wherein the trehalose is present in the isothermal amplification process and/or the reagent composition in an amount of about 3% to about 15% w/v.
D10. The isothermal amplification process of any one of C-D9, wherein the trehalose is present in the isothermal amplification process and/or the reagent composition in an amount of about 3% to about 12% w/v.
D11. The isothermal amplification process of any one of C-D10, wherein the trehalose is present in the isothermal amplification process and/or the reagent composition in an amount of about 3% to about 10% w/v.
D12. The isothermal amplification process of any one of C-D11, wherein the trehalose is present in the isothermal amplification process and/or the reagent composition in an amount of about 3% to about 6% w/v.
D12-1. The present disclosure provides an isothermal amplification process for amplifying a target nucleic acid comprising contacting the target nucleic acid with a reagent composition comprising from about 5% to about 15% w/v of trehalose and from about 2% to about 8% weight of polyethylene glycol (PEG) to amplify the target nucleic acid.
D13. The isothermal amplification process of any one of A-D12-1, wherein the PEG has a molecular weight between about 20,000 Daltons to about 50,000 daltons.
D14. The isothermal amplification process of any one of A-D13, wherein the isothermal amplification process is selected from the group consisting of rolling circle amplification (RCA), nucleic acid sequence-based amplification (NASBA), strand displacement amplification (SDA), Transcription-Mediated Amplification (TMA), Single Primer Isothermal Amplification (SPIA), Helicase-dependent amplification (HDA), Loop mediated amplification (LAMP), Recombinase-Polymerase Amplification (RPA) and Nicking Enzyme Amplification Reaction (NEAR).
D15. The isothermal amplification process of D14, wherein the isothermal amplification process is RPA.
D16. The isothermal amplification process of D14, wherein the isothermal amplification process is NEAR.
D17. The isothermal amplification process of any one of A-D16, wherein the target nucleic acid is a bacterial, eukaryotic or viral nucleic acid.
D18. The isothermal amplification process of any one of A-D17, wherein the target nucleic acid is derived from SARS-COV-2 (COVID-19), HIV-1, HIV-2, HBV, HCV, CMV, Parvovirus B19, HAV, Chlamydia, Gonorrhea, WNV, Zika Virus, Dengue Virus, Chikungunya Virus, Influenza, Babesia, Malaria, Usutu Virus or HEV.
D18-1. The isothermal amplification process of any one of A-D18, wherein the target nucleic acid is derived from HIV-1.
D18-2. The isothermal amplification process of any one of A-D18, wherein the target nucleic acid is derived from HIV-2.
D18-3. The isothermal amplification process of any one of A-D18, wherein the target nucleic acid is derived from HBV.
D18-4. The isothermal amplification process of any one of A-D18, wherein the target nucleic acid is derived from HCV.
D18-5. The isothermal amplification process of any one of A-D18, wherein the target nucleic acid is derived from CMV.
D18-6. The isothermal amplification process of any one of A-D18, wherein the target nucleic acid is derived from Parvovirus B19.
D18-7. The isothermal amplification process of any one of A-D18, wherein the target nucleic acid is derived from HAV.
D18-8. The isothermal amplification process of any one of A-D18, wherein the target nucleic acid is derived from Chlamydia.
D18-9. The isothermal amplification process of any one of A-D18, wherein the target nucleic acid is derived from Gonorrhea.
D18-10. The isothermal amplification process of any one of A-D18, wherein the target nucleic acid is derived from WNV.
D18-11. The isothermal amplification process of any one of A-D18, wherein the target nucleic acid is derived from the Zika Virus.
D18-12. The isothermal amplification process of any one of A-D18, wherein the target nucleic acid is derived from the Dengue Virus.
D18-13. The isothermal amplification process of any one of A-D18, wherein the target nucleic acid is derived from the Chikungunya Virus.
D18-14. The isothermal amplification process of any one of A-D18, wherein the target nucleic acid is derived from Influenza.
D18-15. The isothermal amplification process of any one of A-D18, wherein the target nucleic acid is derived from Babesia.
D18-16. The isothermal amplification process of any one of A-D18, wherein the target nucleic acid is derived from Malaria.
D18-17. The isothermal amplification process of any one of A-D18, wherein the target nucleic acid is derived from the Usutu Virus.
D18-18. The isothermal amplification process of any one of A-D18, wherein the target nucleic acid is derived from HEV.
D19. The isothermal amplification process of any one of A-D18-18, wherein the isothermal amplification process amplifies at least two target nucleic acids or at least three target nucleic acids, e.g., in a multiplex reaction.
D19-1. The isothermal amplification process of D19, wherein the target nucleic acid is derived from the Chikungunya Virus and the Dengue Virus.
D19-2. The isothermal amplification process of D19, wherein the target nucleic acid is derived from HIV-1, HIV-2, HBV and HCV.
D20. The isothermal amplification process of any one of A-D19-2, wherein the target nucleic acid is present in a sample.
D21. The isothermal amplification process of D20, wherein the sample is a tissue sample, and the target nucleic acid is isolated from the tissue sample prior to amplification.
D22. The isothermal amplification process of D20, wherein the sample is a biological fluid.
D23. The isothermal amplification process of D22, wherein the biological fluid is blood.
D24. The isothermal amplification process of D22 or D23, wherein the target nucleic acid is isolated from the biological fluid prior to amplification.
D25. The isothermal amplification process of any one of C-D24, further comprising contacting the target nucleic acid with one or more of a DNA polymerase, a recombinase loading protein, a single stranded binding protein, ATP, dNTPs or a mixture of dNTPs and ddNTPs, a reducing agent, creatine kinase, a nuclease (e.g., an exonuclease), one or more primers, a nucleic acid probe, a reverse transcriptase and an activator.
D26. The isothermal amplification process of any one of C-D24, wherein the reagent composition further comprises one or more of a DNA polymerase, a recombinase loading protein, a single stranded binding protein, ATP, dNTPs or a mixture of dNTPs and ddNTPs, a reducing agent, creatine kinase, a nuclease (e.g., an exonuclease), one or more primers, a nucleic acid probe and a reverse transcriptase.
D27. The isothermal amplification process of any one of A-D26 further comprising detecting the target nucleic acid (e.g., if present).
D28. The isothermal amplification process of D27, wherein detection of the target nucleic acid (e.g., if present) comprises an optical detection process.
E. The present disclosure provides a composition for performing an isothermal amplification process comprising trehalose and PEG, wherein trehalose and PEG are present in the composition at a weight percent (wt %) ratio of about 0.79 or less.
E1. The composition of E, wherein the trehalose to PEG wt % ratio is about 0.75 or less.
E2. The composition of E or E1, wherein the trehalose to PEG wt % ratio is about 0.25 to about 0.79.
E3. The composition of any one of E-E2, wherein the trehalose to PEG wt % ratio is about 0.30 to about 0.75.
E4. The composition of any one of E-E3, wherein the trehalose to PEG wt % ratio is about 0.45 to about 0.75.
E5. The composition of any one of E-E4, wherein the trehalose to PEG wt % ratio is about 0.50 to about 0.70.
E6. The composition of any one of E-E5, wherein the trehalose to PEG wt % ratio is about 0.50 to about 0.60.
E7. The composition of any one of E-E6, wherein the PEG is present in the isothermal amplification process and/or the composition in an amount of about 2% to about 30% weight to volume (w/v).
E8. The composition of any one of E-E7, wherein the PEG is present in the isothermal amplification process and/or the composition in an amount of about 2% to about 25% weight to volume (w/v).
E9. The composition of any one of E-E8, wherein the PEG is present in the isothermal amplification process and/or the composition in an amount of about 5% to about 25% weight to volume (w/v).
E10. The composition of any one of E-E9, wherein the PEG is present in the isothermal amplification process and/or the composition in an amount of about 10% to about 20% weight to volume (w/v).
E11. The composition of any one of E-E10, wherein the PEG is present in the isothermal amplification process and/or the composition in an amount of about 2% to about 8% weight to volume (w/v).
E12. The composition of any one of E-E11, wherein the trehalose is present in the isothermal amplification process and/or the composition in an amount of about 3% to about 20% w/v.
E13. The composition of any one of E-E12, wherein the trehalose is present in the isothermal amplification process and/or the composition in an amount of about 3% to about 15% w/v.
E14. The composition of any one of E-E13, wherein the trehalose is present in the isothermal amplification process and/or the composition in an amount of about 3% to about 12% w/v.
E15. The composition of any one of E-E14, wherein the trehalose is present in the isothermal amplification process and/or the composition in an amount of about 3% to about 5% w/v.
E16. The composition of E15, wherein the trehalose is present in the isothermal amplification process and/or the composition in an amount of about 4% w/v.
E17. The composition of any one of E-E16, wherein the PEG has a molecular weight between about 20,000 Daltons to about 50,000 daltons.
E18. The composition of any one of E-E17, further comprising one or more of a DNA polymerase, a recombinase, a recombinase loading protein, a single stranded binding protein, ATP, dNTPs or a mixture of dNTPs and ddNTPs, a reducing agent, creatine kinase, a nuclease, a nucleic acid probe and a reverse transcriptase.
E19. The composition of any one of E-E18, further comprising one or more of ATP, dNTPs or a mixture of dNTPs and ddNTPs and a reducing agent.
E20. The composition of any one of E-E19, further comprising ATP, dNTPs, phosphocreatine and one or more salts.
E21. The composition of any one of E-E20, wherein the isothermal amplification process is selected from the group consisting of rolling circle amplification (RCA), nucleic acid sequence-based amplification (NASBA), strand displacement amplification (SDA), Transcription-Mediated Amplification (TMA), Single Primer Isothermal Amplification (SPIA), Helicase-dependent amplification (HDA), Loop mediated amplification (LAMP), Recombinase-Polymerase Amplification (RPA) and Nicking Enzyme Amplification Reaction (NEAR).
E22. The composition of E21, wherein the isothermal amplification process is RPA.
E23. The composition of E21, wherein the isothermal amplification process is NEAR.
F. The present disclosure provides a composition for use in an isothermal amplification process comprising trehalose, PEG, ATP, dNTPs, phosphocreatine and one or more salts, wherein trehalose and PEG are present in the composition at a weight percent (wt %) ratio of about 0.79 or less.
G. The present disclosure provides a composition for use in an isothermal amplification process comprising trehalose, PEG, ATP, dNTPs, phosphocreatine and one or more salts, wherein trehalose and PEG are present in the composition at a weight percent (wt %) ratio of about 0.75 or less.
H. The present disclosure provides a composition for use in an isothermal amplification process comprising trehalose, PEG, ATP, dNTPs, phosphocreatine and one or more salts, wherein trehalose and PEG are present in the composition at a weight percent (wt %) ratio of from about 0.25 to about 0.79.
H1. The composition of F, G or H, wherein the trehalose to PEG wt % ratio is about 0.30 to about 0.60.
I. The present disclosure provides a composition for use in an isothermal amplification process consisting essentially of trehalose, PEG, ATP, dNTPs, phosphocreatine and one or more salts, wherein trehalose and PEG are present in the composition at a weight percent (wt %) ratio of from about 0.25 to about 0.79.
I1. The composition of I, wherein the trehalose to PEG wt % ratio is about 0.30 to about 0.60.
J. The present disclosure provides a system for performing the isothermal amplification process of any one of A-D28.
K. The present disclosure provides a system for performing an isothermal amplification process comprising a container or reservoir comprising the composition of any one of E-I1.
K1. The system of J or K, wherein the system is automated.
L. The present disclosure provides a kit for performing the isothermal amplification process of any one of A-D28.
M. The present disclosure provides a kit comprising the composition of any one of E-I1.

### EXAMPLES

The presently disclosed subject matter will be better understood by reference to the following example, which is provided as exemplary of the presently disclosed subject matter, and not by way of limitation.

### Example 1: The Combination of Trehalose and PEG Improves RPA Amplification

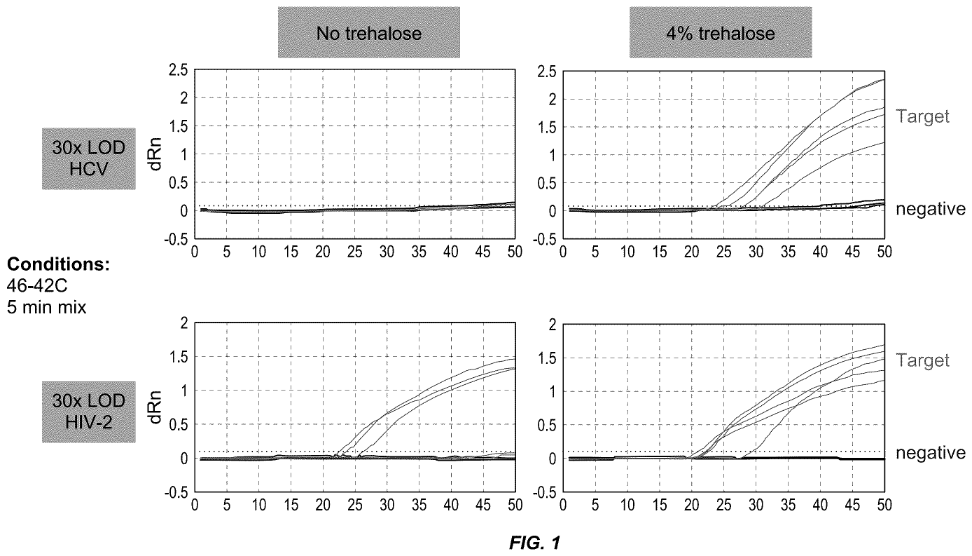

This example discloses the addition of trehalose and PEG in an RPA reaction. FIG. 1 illustrates exemplary results associated with RPA implementations of the disclosed subject matter measured by a real-time fluorescence detection system monitoring fluorescent signal (dRn) over time (cycle). RPA reactions were set up as described herein using the PEG and trehalose concentrations identified in Table 1 and performed at 40 °C.

The addition of 4% trehalose in combination with polyethylene glycol (PEG) improved the detection of HCV (FIG. 1). For example, the 4% trehalose and PEG can be added to the final reaction from a composition that includes the non-protein components used in an RPA reaction (referred to herein as “NPC”). When HCV was present at a level of 30× the limit of detection (LOD), the signal was detected between 20 and 30 cycles with 4% trehalose and not detected using a master mix that did not contain trehalose (FIG. 1). Without being limited to a particular theory, trehalose can promote the relaxation of the target nucleic acid which can facilitate binding of the enzymes and/or the primers to the target amplicon. Without being limited to a particular theory, trehalose could also reduce the melting temperature (Tm) of the target DNA/RNA, increase reaction crowding and improve the thermal stability of enzymes. This improved amplification was also observed for other HxV and IC targets (data not shown) and was observed on both the NOVA automated (ADBB) and Bio-Rad systems. HxV is a multiplex assay that detects HIV-1, HIV-2, HBV and HCV nucleic acids.

TABLE 1

| | NPC (0.0) Trehalose/PEG | NPC (0.73) Trehalose/PEG |
|---|---|---|
| Reagent | ratio | ratio |
| Trehalose | 0.0% | 4.0% |
| PEG | 5.5% | 5.5% |

NPC = Non-protein component composition

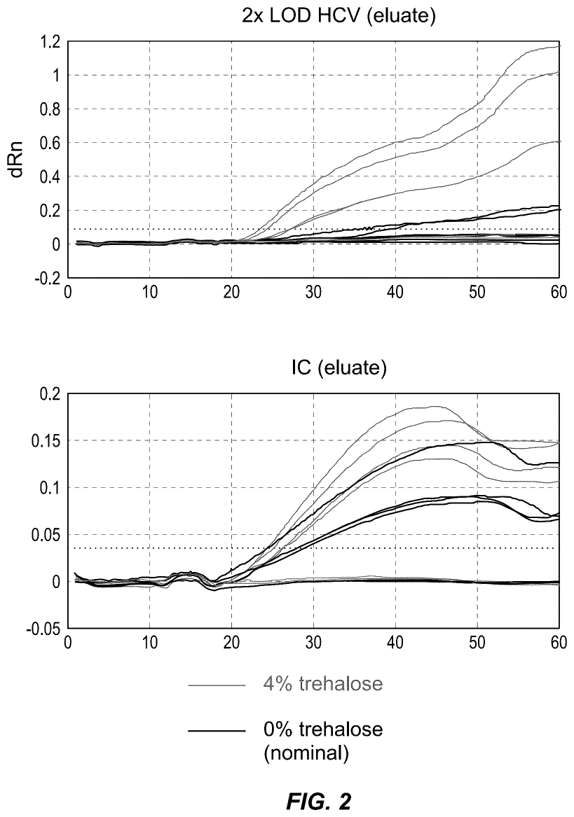

FIG. 2 further illustrates exemplary results demonstrating the improved amplification with the addition of 4% wt trehalose. RPA reactions were set up as described herein using the PEG and trehalose concentrations identified in Table 2 and performed at 40 °C. The addition of 4% wt trehalose to the final reaction from the NPC improved the detection of HCV target nucleic acids at near limit of detection levels (i.e., 2×LOD) on the Bio-Rad system (FIG. 2). Improved amplification was also observed for other HxV and IC target nucleic acids (FIG. 2 and data not shown).

TABLE 2

| | NPC Trehalose/PEG | NPC Trehalose/PEG |
|---|---|---|
| Reagent | ratio = 0 | ratio = 0.62 |
| Trehalose | 0.0 (nominal) wt % | 4.0 wt % |
| PEG | 6.5 wt % | 6.5 wt % |

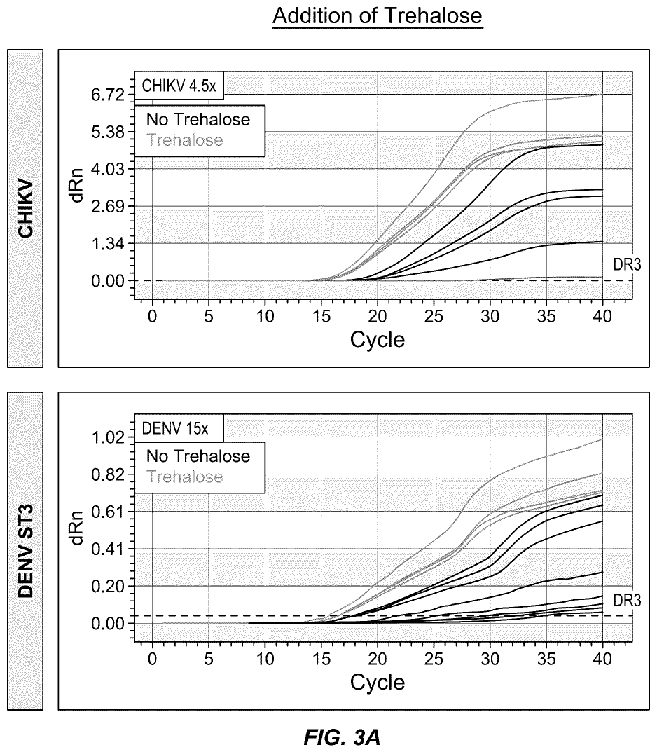

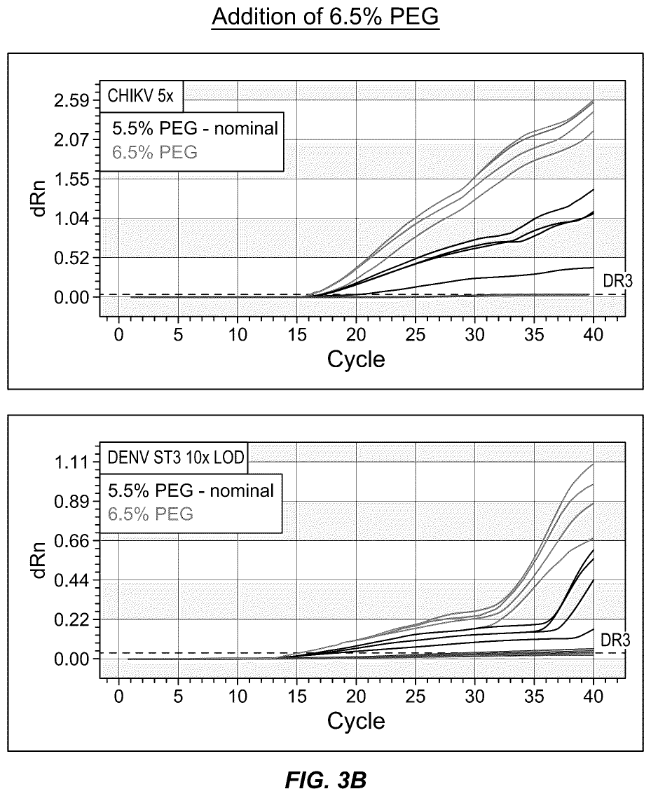

FIGS. 3A and 3B illustrate exemplary results associated with various trehalose/PEG ratios. RPA reactions were set up as described herein using the PEG and trehalose concentrations identified in Tables 3A and 3B and performed at 40 °C. The PEG and trehalose concentrations provided in Tables 3A and 3B represent the concentrations of trehalose and PEG in the final reaction that results from the addition of trehalose and PEG from the NPC to the final reaction. The addition of trehalose to result in a final reaction concentration of 4% wt trehalose obtained from the NPC improved the RPA amplification signal of Chikungunya and Dengue target nucleic acids (FIG. 3A). Increasing the concentration of PEG from 5.5% wt to 6.5% wt improved the RPA amplification signal of Chikungunya and Dengue target nucleic acids (FIG. 3B).

TABLE 3A

| | NPC Trehalose/PEG | NPC Trehalose/PEG |
|---|---|---|
| Reagent | ratio = 0 | ratio = 0.73 |
| Trehalose | 0.0 wt % | 4.0 wt % |
| PEG | 6.5 wt % | 5.5 wt % |

TABLE 3B

| Reagent | NPC Trehalose/PEG ratio = 0 | | NPC Trehalose/PEG ratio = 0 | |
|---|---|---|---|---|
| Trehalose | 0 | wt % | 0 | wt % |
| PEG | 5.5 | wt % | 6.5 | wt % |

FIG. 4 illustrates exemplary results associated with various trehalose/PEG ratios. RPA reactions were set up as described herein using the PEG and trehalose concentrations identified in Table 4 and performed at 40 °C. The 1.04 trehalose/PEG ratio improved the sensitivity and signal strength of HCV at 2×LOD on the NOVA automated system (FIG. 4). However, other HxV targets were not impacted by the higher concentration of trehalose (data not shown).

TABLE 4

| | NPC Trehalose/PEG | NPC Trehalose/PEG |
|---|---|---|
| Reagent | ratio = 0.52 | ratio = 1.04 |
| Trehalose | 4.0 wt % | 8.0 wt % |
| PEG | 7.7 wt % | 7.7 wt % |

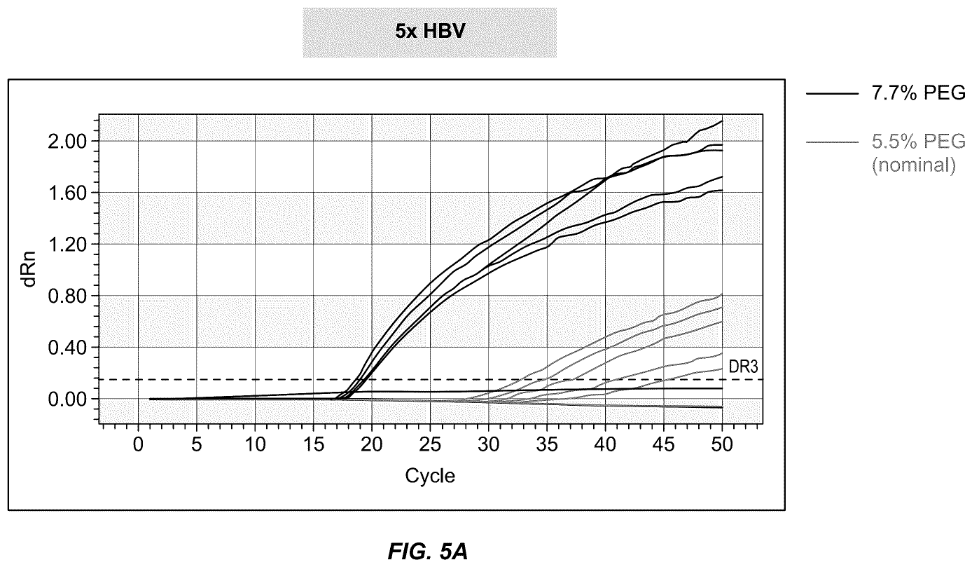

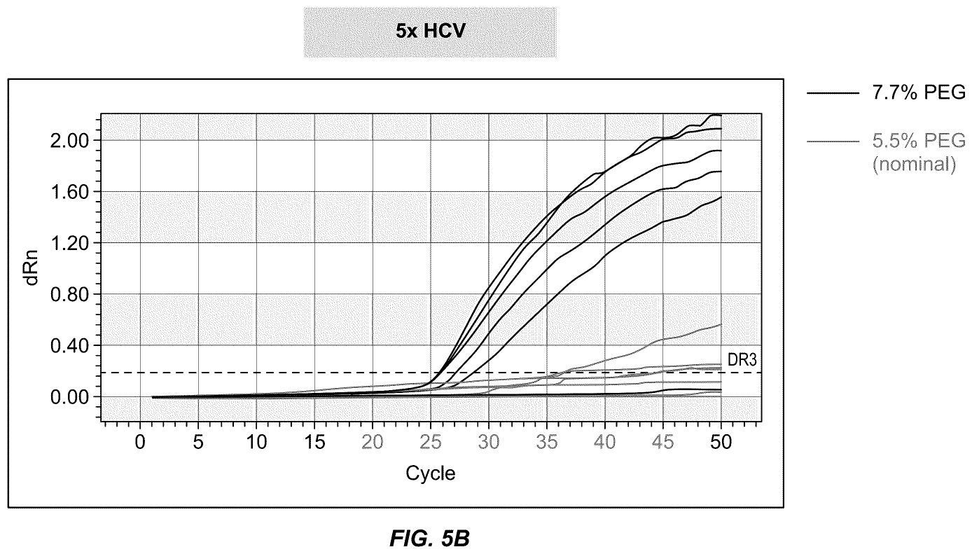

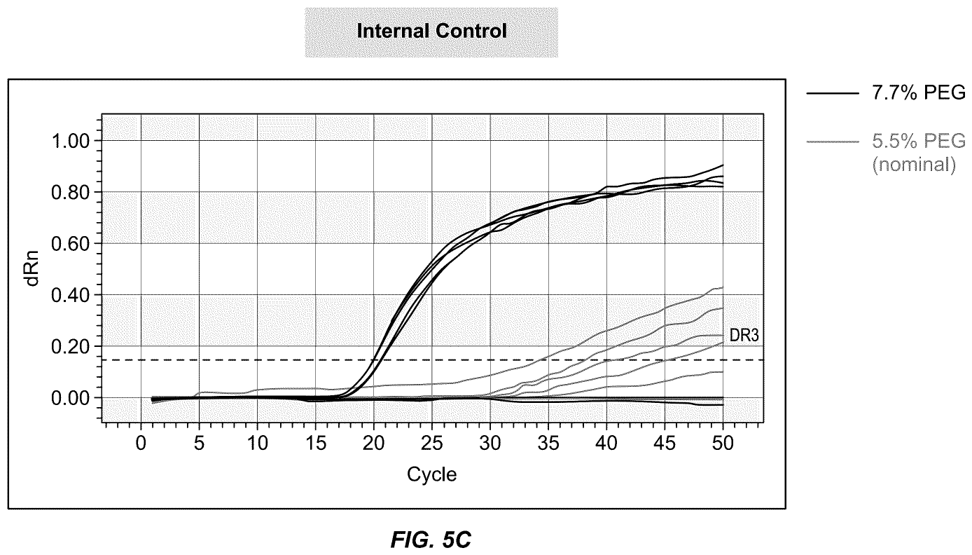

FIGS. 5A-5C illustrate exemplary results associated with various trehalose/PEG ratios. RPA reactions were set up as described herein using the PEG and trehalose concentrations identified in Table 5 and performed at 40 °C. The 0.52 Trehalose/PEG ratio in the NPC significantly improved the detection of multiplex HxV targets including HBV, HCV and Internal Control (FIGS. 5A-5C). The improved amplification was observed across multiple targets and was most pronounced for HCV, which was detected at under 30 cycles using the reaction mixture containing trehalose and PEG at a wt % ratio of 0.52 (FIG. 5B).

TABLE 5

| | NPC Trehalose/PEG | NPC Trehalose/PEG |
|---|---|---|
| Reagent | ratio = 0.73 | ratio = 0.52 |
| Trehalose | 4.0 wt % | 4.0 wt % |
| PEG | 5.5 wt % | 7.7 wt % |

The 0.52 trehalose/PEG wt % ratio in the NPC was found to significantly improve amplification on the NOVA automated system. In particular, the combination of 4% wt trehalose and 7.7% wt PEG in the final reaction that results from the addition of trehalose and PEG from the NPC to the final reaction, which corresponds to a trehalose/PEG wt % ratio of 0.52 in the NPC, was identified as providing the best amplification of all HxV targets. This example shows that specific trehalose to PEG ratios and amounts significantly improve amplification of target nucleic acids.

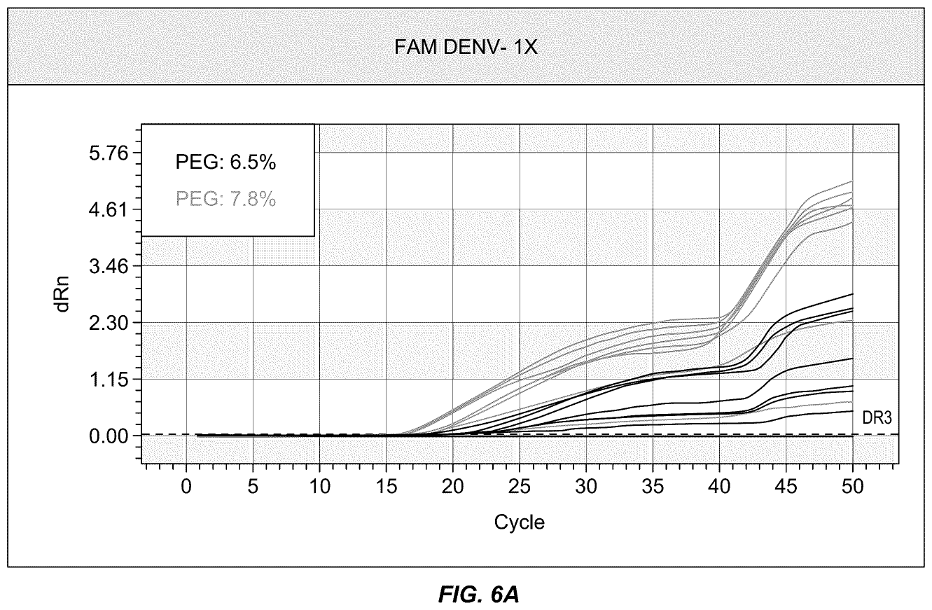

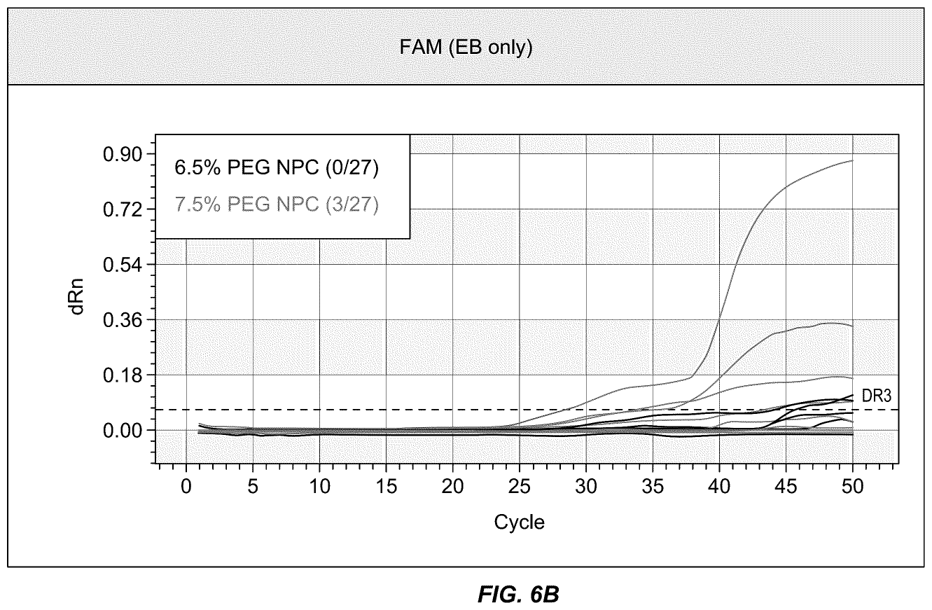

FIGS. 6A-6B illustrate exemplary results associated with various trehalose/PEG ratios. RPA reactions were set up as described herein using the PEG and trehalose concentrations identified in Table 6 and performed at 40 °C. The concentrations provided in Table 6 represent the concentrations of trehalose and PEG in the final reaction that results from the addition of trehalose and PEG from the NPC to the final reaction.

TABLE 6

| | NPC Trehalose/PEG | | NPC Trehalose/PEG |
|---|---|---|---|
| Reagent | ratio = 0.31 | | ratio = 0.26 |
| Trehalose | 2.0 wt % (when | 5.96 wt % | 2.0 wt % (when |
| | diluted in final | in NPC | diluted in final |
| | reaction) | | reaction) |
| PEG | 6.5 wt % (when | 17.88 wt % | 7.8 wt % (when |
| | diluted in final | in NPC | diluted in final |
| | reaction) | | reaction) |

The 0.26 trehalose/PEG in the NPC improved the detection of DENV targets (FIG. 6A) but increased the number of false positives (FIG. 6B). The 0.31 trehalose/PEG wt % ratio was found to significantly improve amplification on the NOVA automated system while producing zero false positives. In particular, the combination of 2% trehalose and 6.5% PEG in the final concentration obtained from the NPC, which resulted in a trehalose/PEG wt % ratio of 0.31, was identified as providing increased amplification of DENV targets without producing false positives. This example shows that specific trehalose to PEG ratios and amounts significantly improve amplification of target nucleic acids while maintaining the specificity of the assay.

Although the presently disclosed subject matter and its advantages have been described in detail, it should be understood that various changes, substitutions and alterations can be made herein without departing from the spirit and scope of the present disclosure. Moreover, the scope of the present application is not intended to be limited to the particular embodiments of the process, machine, manufacture, and composition of matter, means, methods and steps described in the specification. Accordingly, the appended claims are intended to comprise within their scope such processes, machines, manufacture, compositions of matter, means, methods or steps.

Various patents, patent applications, publications, product descriptions and protocols are cited throughout this application, the disclosure of which are incorporated herein by reference in their entireties for all purposes.

1-81. (canceled)

82. An isothermal amplification process for amplifying a target nucleic acid, wherein the isothermal amplification process comprises:
(i) providing a reagent composition, wherein the reagent composition is prepared using a composition comprising trehalose and a polyethylene glycol (PEG), and wherein the trehalose and PEG are present in the composition at a trehalose to PEG weight percent (wt %) ratio of about 0.79 or less; and
(ii) contacting the target nucleic acid with the reagent composition to amplify the target nucleic acid.

83. The isothermal amplification process of claim 82, wherein the trehalose to PEG wt % ratio is about 0.25 to about 0.79, about 0.25 to about 0.75, about 0.3 to about 0.70 or about 0.3 to about 0.60.

84. The isothermal amplification process of claim 82, wherein the trehalose to PEG wt % ratio is about 0.30 to about 0.55.

85. The isothermal amplification process of claim 82, wherein (i) the PEG is present in the composition in an amount of about 10% to about 30% weight to volume (w/v) and/or the trehalose is present in the composition in an amount of about 5% to about 20% w/v, and/or (ii) the reagent composition comprises PEG at an amount from about 2% to about 8% weight to volume (w/v) and/or the reagent composition comprises trehalose at an amount of about 3% to about 12% w/v.

86. The isothermal amplification process of claim 82, wherein the PEG is present in the composition in an amount of about 15% to about 20% weight to volume (w/v) and/or the trehalose is present in the composition in an amount of about 5% to about 7% w/v.

87. The isothermal amplification process of claim 82, wherein (i) the composition further comprises one or more of ATP, dNTPs, phosphocreatine and one or more salts, and/or (ii) the reagent composition further comprises one or more of a DNA polymerase, a recombinase loading protein, a single stranded binding protein, ATP, dNTPs or a mixture of dNTPs and ddNTPs, a reducing agent, creatine kinase, a nuclease, one or more primers, a nucleic acid probe and a reverse transcriptase.

88. An isothermal amplification process for amplifying a target nucleic acid comprising contacting the target nucleic acid with a reagent composition comprising trehalose and a polyethylene glycol (PEG) to amplify the target nucleic acid, wherein the trehalose and PEG are in the reagent composition at a weight percent (wt %) ratio of about 0.79 or less.

89. The isothermal amplification process of claim 88, wherein the trehalose to PEG wt % ratio is about 0.3 to about 0.79, about 0.45 to about 0.75, about 0.50 to about 0.70 or about 0.50 to about 0.60.

90. The isothermal amplification process of claim 88, wherein the PEG is present in the reagent composition in an amount of about 2% to about 20% weight to volume (w/v) and/or the trehalose is present in the reagent composition in an amount of about 3% to about 8% w/v.

91. The isothermal amplification process of claim 88, wherein the PEG is present in the reagent composition in an amount of about 2% to about 10% weight to volume (w/v) and/or the trehalose is present in the reagent composition in an amount of about 3% to about 6% w/v.

92. The isothermal amplification process of claim 82, wherein the isothermal amplification process is selected from the group consisting of rolling circle amplification (RCA), nucleic acid sequence-based amplification (NASBA), strand displacement amplification (SDA), Transcription-Mediated Amplification (TMA), Single Primer Isothermal Amplification (SPIA), Helicase-dependent amplification (HDA), Loop mediated amplification (LAMP), Recombinase-Polymerase Amplification (RPA) and Nicking Enzyme Amplification Reaction (NEAR).

93. The isothermal amplification process of claim 92, wherein the isothermal amplification process is RPA.

94. The isothermal amplification process of claim 82, wherein the target nucleic acid is derived from SARS-COV-2 (COVID-19), HIV-1, HIV-2, HBV, HCV, CMV, Parvovirus B19, HAV, Chlamydia, Gonorrhea, WNV, Zika Virus, Dengue Virus, Chikungunya Virus, Influenza, Babesia, Malaria, Usutu Virus or HEV.

95. The isothermal amplification process of claim 82, wherein the isothermal amplification process amplifies at least two target nucleic acids or at least three target nucleic acids.

96. A composition for performing an isothermal amplification process comprising trehalose and PEG, wherein trehalose and PEG are present in the composition at a weight percent (wt %) ratio of about 0.79 or less.

97. The composition of claim 96, wherein the trehalose to PEG wt % ratio is about 0.30 to about 0.75, about 0.45 to about 0.75, about 0.50 to about 0.70 or about 0.50 to about 0.60.

98. The composition of claim 96, wherein the PEG is present in the composition in an amount of about 5% to about 25% weight to volume (w/v) and/or the trehalose is present in the composition in an amount of about 3% to about 15% w/v.

99. The composition of claim 96, wherein the PEG is present in the composition in an amount of about 10% to about 20% weight to volume (w/v) and/or the trehalose is present in the composition in an amount of about 3% to about 8% w/v.

100. The composition of claim 96, further comprising one or more of a DNA polymerase, a recombinase, a recombinase loading protein, a single stranded binding protein, ATP, dNTPs or a mixture of dNTPs and ddNTPs, phosphocreatine, one or more salts, a reducing agent, creatine kinase, a nuclease, a nucleic acid probe and a reverse transcriptase.

101. A composition for use in an isothermal amplification process consisting essentially of trehalose, PEG, ATP, dNTPs, phosphocreatine, buffer and one or more salts, wherein trehalose and PEG are present in the composition at a weight percent (wt %) ratio of about 0.25 to about 0.79.

102. The composition of claim 101, wherein the trehalose to PEG wt % ratio is about 0.30 to about 0.60 or about 0.30 to about 0.55.

103. A system for performing an isothermal amplification process, comprising a container or a reservoir comprising the composition of claim 96.

104. The system of claim 103, wherein the system is automated.

105. A kit comprising the composition of claim 96.
